(19) United States
(12) Patent Application Publication
(10) Pub. No.: US 2026/0308207 A1
(43) Pub. Date: Oct. 8, 2026

(54) SYNTHETIC mRNA FOR TREATING EBV-ASSOCIATED DISEASES

(71) Applicant: The Chinese University of Hong Kong, Shatin, New Territories, Hong Kong SAR (CN)

(72) Inventors: Kwok Wai LO, Ma On Shan, N.T., Hong Kong SAR (CN); Tom Pok Man HAU, Kowloon, Hong Kong SAR (CN); Man WU, Ma On Shan, N.T., Hong Kong SAR (CN); Anna Chi Man TSANG, Shatin, N.T., Hong Kong SAR (CN)

(21) Appl. No.: 19/477,362

(22) PCT Filed: Apr. 25, 2024
(86) PCT No.: PCT/CN2024/089759

§ 371 Date: Oct. 21, 2025

Related U.S. Application Data
(60) Provisional application No. 63/462,197, filed on Apr. 26, 2023.

(51) Int. Cl. C07K 14/195; A61K 38/00; A61P 35/00

(52) U.S. Cl. CPC C07K 14/195; A61P 35/00; A61K 38/00; C07K 2319/09

(57) ABSTRACT

Provided synthesized mRNAs that can specifically induce the transcription of one or more Epstein-Barr virus (EBV) lytic genes in EBV-infected cells, thus destroying the cells. Also described herein are methods of synthesizing the mRNAs as well as methods of using the mRNAs for the purpose of treating EBV-associated diseases such as EBV-positive cancers.

### RELATED APPLICATIONS

This application claims priority to U.S. Provisional Patent Application No. 63/462,197, filed Apr. 26, 2023, the contents of which are hereby incorporated by reference in the entirety for all purposes.

### BACKGROUND OF THE INVENTION

A remarkably wide range of lymphoid malignancies and two distinct types of human epithelial cancer, nasopharyngeal carcinoma (NPC) and EBV-associated gastric cancer (EBVaGC), are driven by the persistent latent infection of Epstein-Barr virus (EBV). Collectively these EBV-associated tumors impose a global disease burden estimated to reach 265, 186 new cases per year at 2017. Among these EBV-associated cancers, greater than 40% are nasopharyngeal carcinoma (NPC) that are prevalent in South China and Southeast Asia. The well-documented virus-cell interaction in tumorigenesis indicates that targeting EBV is an efficient approach to eradicate these cancers. Notably, the unique episomal nature of the EBV genome in the tumor cells implies induction of cell death by reactivation of viral lytic cycle is an attractive approach to cure the EBV-associated cancers. When the latent EBV viruses are induced into lytic cycle, the immediate-early (IE) proteins, BZLF1 and BRLF1 must be expressed and further activated the transcription of early and late proteins to progress. Reactivation of the virus from latency is dependent on expression of the viral BZLF1 and BRLF1 proteins. Earlier studies have demonstrated the usefulness in cancer therapy of a strategy for killing cancer cells harboring viral genomic sequences by way of activating the viral lytic cycle, see, e.g., WO 2021/173977A9. The present disclosure relates to the application of synthetic mRNAs to efficiently activate EBV lytic genes in EBV-latent infected cells. These synthetic nucleoside-modified mRNAs can be packaged into lipid nanoparticles (LNPs) or other non-viral delivery systems and then delivered to the tumor cells to translate an artificial protein containing linked DNA binding and transcriptional activation domains that specifically activates the transcription of EBV-encoded lytic genes such as BZLF1, BRLF1, and BGLF4. The mRNA nanomedicine technology provided herein overcomes the highly complex regulatory mechanisms of EBV lytic gene expression. By taking advantage of the high copy number of EBV episomes in the EBV-infected cancer cells, the synthesized designed mRNAs enable highly efficient BZLF1 and BRLF1 expression to reactivate EBV lytic cycle. This artificial activation of EBV-lytic gene transcription, including transcription of the EBV-encoded protein kinase BGLF4, enhances efficient conversion of the antiviral non-toxic prodrug form of ganciclovir to its cytotoxic DNA replication inhibitor form for rapidly killing the cancer and bystander cells. The present disclosure provides methods of inducing EBV immediately early and early lytic cycle genes with high specificity by synthesized modified mRNAs. These synthetic mRNAs are first-in-class therapeutic drugs slow or stop cancer cell growth in vitro and in vivo, being innovative therapeutic strategies effectively activating viral lytic genes for lytic induction therapy of EBV-associated diseases.

### BRIEF SUMMARY OF THE INVENTION

The invention describes a first-in-class of synthetic mRNA medicines useful in lytic induction therapy for specific killing of EBV-positive cells associated with a condition or disease, especially EBV-positive cancer cells. The synthesized nucleoside-modified mRNAs are designed to effectively activate EBV lytic genes in all EBV-associated cells while the induction of lytic cycle by existing chemical inducers (e.g., HDAC inhibitors, gemcitabine) are cell context specific and with varying levels of efficiency among different pathologies (e.g., different tumors). Currently, the implementation of EBV lytic induction treatment in clinical management of cancer patients are limited by their broad-spectrum cytotoxicity and low efficiency in lytic reactivation. The synthesized nucleoside-modified mRNA effectively activates the EBV lytic promotors, bypassing the highly complex regulatory mechanisms in all EBV-associated cancer cells. Furthermore, the preferential transcription activation ability and cytotoxic to EBV-associated malignant cells, but not normal cells, have been demonstrated in both in vitro and in vivo settings. Instead of DNA-based transcription activation constructs, the synthesized modified-mRNAs show advantage in small in size, high efficiency of package and delivery, and low risk in inducing genome recombination and aberrant immune responses.

In a first aspect, the present invention provides a composition that is useful for treating EBV-associated pathologies such as EBV-associated cancers in a human subject. The composition comprises (1) a nucleic acid comprising a polynucleotide sequence, which encodes a fusion protein comprising (i) at least one nuclear-localization signal (NLS); (ii) a transcription activator-like effector (TALE) targeting Epstein-Barr virus (EBV) BZLF1, BRLF1, or BGLF4 promoter sequence; and (iii) a transactivation domain capable of initiating transcription of the BZLF1, BRLF1, or BGLF4 gene, and (2) one or more physiologically acceptable excipients. In some embodiments, the nucleic acid of the invention as described above and herein is DNA. In some embodiments, the nucleic acid of this invention is RNA. In some embodiments, the uracil (U) residues of the RNA are replaced with pseudouridines (such as N1-methylpseudouridine), at least partially and in some cases entirely. In some embodiments, the nucleic acid is a DNA and comprises an expression cassette comprising this fusion protein-encoding polynucleotide sequence operably linked to a promoter sequence. In some embodiments, the TALE is encoded by a nucleotide sequence having at least 90%, 95%, or up to 100% sequence identity to any one of the segments specified in Table 7 as the TALE of SEQ ID NOs: 31-60, e.g., segment 649-2178 of SEQ ID NO: 31; segment 649-2382 of SEQ ID NO: 32; segment 649-2280 of SEQ ID NO: 33; or segment 649-2281 of SEQ ID NO: 34. In some embodiments, the transactivation domain is encoded by a nucleotide sequence having at least 90%, 95%, or up to 100% sequence identity to segment 2722-3564 of SEQ ID NO: 33. In some embodiments, the NLS is encoded by a nucleotide sequence having at least 90%, 95%, or up to 100% sequence identity to segment 91-114 of SEQ ID NO: 33. In some embodiments, a plurality of NLS (e.g., 3×NLS) is used in the nucleotide sequence, for example, segment 91-162 of SEQ ID NO: 33. Additional examples of coding sequences for TALE, transaction domain, and NLS can be found in SEQ ID NOs: 31-60 as marked in Tables 4-6. In some embodiments, the fusion protein comprises an epitope tag, e.g., a FLAG tag. For instance, one exemplary fusion protein comprises, from its N-terminus to its C-terminus, optionally a FLAG encoded by segment 1-69 of SEQ ID NO: 33, at least one NLS (e.g., 3×NLS) encoded by segment 91-162 of SEQ ID NO: 33, a TALE encoded by segment 649-2280 of SEQ ID NO: 33, and a transactivation domain encoded by segment 2722-3564 of SEQ ID NO: 33. Exemplary polynucleotide sequences encoding the fusion protein of this invention are presented in SEQ ID NOs: 31-60, with their functional segments marked as shown in Tables 4-6. Moreover, the NLS, TALE, and transactivation domain coding sequences as marked and shown in Tables 4-6 in each of SEQ ID NOs: 31-60 may be individually chosen to form one or more additional NLS-TALE-transactivation domain combinations in order to produce new polynucleotide sequences that encode fusion proteins useful for effectively activating EBV lytic genes in all EBV-associated cells and causing lysis of the cells. In particular, additional fusion proteins of this utility based on additional TALE-transactivation domain pairings can be made by selecting and combining coding sequences for TALE and for transactivation domain taken from within the group of sequences set forth in SEQ ID NOs: 31-40, or similarly from within the group of sequences set forth in SEQ ID NOs: 41-50 or the group of sequences set forth in SEQ ID NOs: 51-60. For example, any one of the TALE coding sequences as shown in segment 649-2178 of SEQ ID NO: 31, segment 649-2382 of SEQ ID NO: 32, segment 649-2280 of SEQ ID NO: 33, and segment 649-2281 of SEQ ID NO: 34 may be used interchangeably in combination with other NLS and/or transactivation domain coding sequences (especially those from SEQ ID NOs:

31-40) to generate additional fusion proteins for activating EBV lytic genes. In some embodiments, the composition of this invention is specifically formulated for use in treating an EBV-associated disease in a subject in need thereof, for example, the nucleic acid, which may be RNA or DNA as described above and herein is present within lipid nanoparticles. In some embodiments, the lipid nanoparticles comprise [(4-hydroxybutyl) azanediyl] di (hexane-6,1-diyl) bis(2-hexyldecanoate) (ALC-0315), 1, 2-distearoyl-sn-glycero-3-phosphocholine (DSPC), 1,2-dimyristoyl-rac-glycero-3-methoxypolyethylene glycol-2000 (DMG-PEG 2000), and cholesterol. For a nucleic acid in the form of RNA, at least some potentially all of the U residues of the molecule are replaced with pseudouridine, e.g., for the purpose of improving the stability and/or bioavailability of the RNA molecule, thus ensuring adequate expression of the fusion protein encoded by the RNA. In some embodiments, the composition is formulated for injection, e.g., in the form of a liquid, solution, suspension, or emulsion. In some embodiments, the EBV-associated cancer is Burkitt lymphoma, Hodgkin's lymphoma, natural killer cell lymphoma, T-cell lymphoma, post-transplant lymphoma, nasopharyngeal carcinoma, or gastric cancer.

In the second aspect, the present invention provides a method for treating an EBV-associated pathology such as an EBV-associated cancer in a subject in need thereof by administering to the subject an effective amount of the composition described above and herein, namely containing an effective amount of (1) a nucleic acid described above or herein; and (2) one or more physiologically acceptable excipient s. In some embodiments, the nucleic acid is RNA, which optionally has at least some, possibly all of its U residues replaced with pseudouridine (e.g., N1-methylpseudouridine), for improved stability and/or bioavailability. Regardless of the nucleic acid being a DNA or RNA molecule, in some embodiments it is present in the composition within lipid nanoparticles. According to the present invention, the treatment method in some embodiments is practiced by administering to the subject the composition of this invention by systemic administration, e.g., by intravenous, intratumoral, intramuscular, or subcutaneous injection, or by oral ingestion or by nasal inhalation. In some embodiments, the EBV-associated cancer is Burkitt lymphoma, Hodgkin's lymphoma, natural killer cell lymphoma, T-cell lymphoma, post-transplant lymphoma, nasopharyngeal carcinoma, or gastric cancer.

In a related aspect, the present invention provides a novel use of a composition for treating an EBV-associated pathology (such as an EBV-associated cancer) in a subject. The composition contains an effective amount of (1) a nucleic acid de scribed above or herein; and (2) one or more physiologically acceptable excipients. In some embodiments, the nucleic acid is RNA, which optionally has at least some, possibly all of its U residues replaced with pseudouridine (e.g., N1-methylpseudouridine). Regardless of the nucleic acid being a DNA or RNA molecule, in some cases it is present in the composition within lipid nanoparticles. Depending on the mood of administration, in some embodiments the composition of this invention is formulated for systemic administration, e.g., in a liquid or semi-liquid form for injection intravenously, intratumoral, intramuscularly, or subcutaneously, or in a powder or aerosolized form for nasal inhalation, or in a liquid/semi-liquid or solid/semi-solid form such as a solution, emulsion, paste, cream, powder, tablet, or capsule for oral ingestion. In some embodiments, the EBV-associated cancer is Burkitt lymphoma, Hodgkin's lymphoma, natural killer cell lymphoma, T-cell lymphoma, post-transplant lymphoma, nasopharyngeal carcinoma, or gastric cancer.

In a third aspect, the present invention provides a kit for treating EBV-associated pathologies such as EBV-associated cancers in patients in need thereof. Typically, the kit includes a plurality of containers, a first container containing a first composition comprising an effective amount of the nucleic acid described above and herein, a second container containing a second composition comprising an effective amount of at least one therapeutic agent that is known to be effective for treating the EBV-associated pathology, e.g., an anti-cancer therapeutic agent. In some embodiments, the nucleic acid is RNA, which optionally with at least some, possibly all of its U residues replaced with pseudouridine (such as N1-methylpseudouridine). In some embodiments, the polynucleotide sequence is set forth in any one of SEQ ID NOs: 31-60. In some embodiments, the nucleic acid is RNA and the polynucleotide sequence is set forth in any one of SEQ ID NOs: 51-60, and the other anti-cancer agent comprises ganciclovir (GCV). In some embodiments, the nucleic acid is presented in lipid nanoparticles, e.g., comprising [(4-hydroxybutyl) azanediyl] di (hexane-6,1-diyl) bis(2-hexyldecanoate) (ALC-0315), 1,2-distearoyl-sn-glycero-3-phosphocholine (DSPC), 1,2-dimyristoyl-rac-glycero-3-methoxypolyethylene glycol-2000 (DMG-PEG 2000), and cholesterol. In some embodiments, the EBV-associated cancer is Burkitt lymphoma, Hodgkin's lymphoma, natural killer cell lymphoma, T-cell lymphoma, post-transplant lymphoma, nasopharyngeal carcinoma, or gastric cancer.

### BRIEF DESCRIPTION OF THE DRAWINGS

FIG. 1. Reactivation of EBV lytic genes in EBV-associated cancers via CRISPR-based transcriptional activators. FIG. 1a, quantitative RT-PCR demonstrates the endogenous expression of Zta/BZLF1, Rta/BRLF1 and BGLF4 in SNU719 and C666-1 cells at 24 h after transient transfection with HA-dCas9-2A-EGFP, 3xFLAG-PUFa-p65HSF1, and sgRNAs. FIG. 1b, western blotting (dem onstrates the endogenous expression of Zta/BZLF1, Rta/BRLF1 and BGLF4 in SNU719 and C666-1 cells at 24 h after transient transfection with HA-dCas9-2A-EGFP, 3xFLAG-PUFa-p65HSF1, and sgRNAs (n=3/group). Data are presented as mean±SD. ***: P<0.001. FIG. 1c, In SNU719, C666-1 and C17 cells stably transfected with sgRNA3, HA-dCas9-2A-EGFP, and inducible 3xFLAG-PUFa-p65HSF1 transactivator, the expression of Zta and other EBV lytic proteins (Rta, BGLF4 and VCAp18) were detected by western blotting aft er doxycycline (Dox) treatment for 48 h (n=3/group). FIG. 1d, Immunofluorescence staining illustrated the expression of Zta, EA-D and gp350 lytic proteins in Dox-treated stably transfected SNU719, C666-1 and C17 cells (scale bar=10 mm) (n=3/group). FIG. 1e, Using flow cytometry analysis, the percentage of Zta-expressing cells was determined in the stably transfected SNU719, C666-1 and C17 cells at 48 h post-Dox treatment. Representative flow cytometry plots are shown (n=3/group). FIG. 1f, Inhibition of cell viability was detected in Dox-treated stably transfected SNU719, C666-1 and C17 cells using the CCK8 assay (n=3/group). FIG. 1g, BZLF1 induction significantly suppressed the colony formation ability of Dox-treated stably transfected EBV-positive cancer cells (n=3/group). Data are presented as mean±S D. **: P<0.01; ***: P<0.001.

FIG. 2. Synthetic BZLF1-specific TALE transcriptional activator. FIG. 2a, Western blotting detected the endogenous expression of immediate early (Zta, Rta), early (BGLF4), and late (VCAp18) lytic proteins in SNU719 and C666-1 cells transiently transfected with the designed BZLF1-targeted TALE plasmid, TZ3 and other TALE plasmids, TZ1, TZ2 and TZ4. P3HR1 cells treated with NaB and TPA were included as the controls for lytic protein expression (n=3/group). FIG. 2b, Using immunofluorescence (IF) staining, Zta expression was detected in SNU719 and C666-1 cells transiently transfected with TZ3 (n=3/group). Representative IF images are shown. FIG. 2c, The promoter sequences of wild-type EBV BZLF1 (B95-8, Zp-P) and reported variants (Zp-V3, Zp-V4 and Zp-V1) are shown. Cis-regulatory element sequences and sequence variations are shown in capital letters and red font, respectively. The Z3-binding sequence is highlighted in yellow and is conserved among all EBV strains. FIG. 2d, Detection of the specific binding ability of the TZ3 transcriptional activator to the targeted sequence in the BZLF1 promoter in C666-1 cells by EMSA (arrow). In addition to a probe specific for the wild-type (WT) target sequence in the BZLF1 promoter, three mutant probes were included. Their sequences are shown in the box. Cells transfected with vector alone were used as the control (n=3/group). FIG. 2e, Using RNA sequencing, differentially expressed genes between TZ3-transfected and control HK1 cells were determined (n=3/group). Few genes showed significant changes in expression in TZ3-transfected HK1 cells. FIG. 2f, No significant reduction of cell viability was detected in the TZ3-transfected HK1 cells (n=3/group). Data are presented as mean±SD. ns: not significant, P>0.05). FIG. 2g, Ectopic transfection of TZ3 did not significantly alter the cell cycle of HK1 cells (n=3/group). Data are presented as mean±SD. ns: not significant.

FIG. 3. Reactivation of EBV lytic genes by LNP-encapsulated nucleoside-modified mRNA (mTZ3-LNP). FIG. 3a, The particle size, polydispersity index, and zeta potential of mTZ3-LNP were analyzed using a dynamic light scattering method. The data are representative of four independent experiments. FIG. 3b, The cell uptake process of the LNP-encapsulated Cy5-labeled mTZ3 mRNAs by SNU719 cells over 1, 3 and 6 h was visualized using an LSM 880 confocal laser scanning microscope. The fluorescence signals were measured in three channels: Cy5, excitation/emission wavelength, (ex/em) 633/697 nm; Dnd-26, ex/em, 488/524 nm; and hoechst, ex/em 405/460 nm. FIG. 3c, Western blotting was used to detect Zta expression in SNU719 and C666-1 cells treated with mTZ3-LNPs and LNP-encapsulated control mRNA (control-LNP) for 24 h. The EBV-negative NPC cell line HK1 was used as the negative control. FIG. 3d, Western blotting detected the expression of Zta, Rta, the downstream early (BGLF4) and late (VCA) lytic proteins and cleaved caspase 3 in SNU719 and C666-1 cells treated with mTZ3-LNPs for 3 to 96 h. FIG. 3e, Immunofluorescence staining and (FIG. 3f) flow cytometry analysis illustrated the induction of Zta, EA-D and gp350 expression in mTZ3-LNP-treated SNU719 and C666-1 cells.

FIG. 4. Highly efficient EBV lytic reactivation in a panel of EBV-positive cancer cells treated with mTZ3-LNPs. Representative flow cytometry plots show the high efficiency of mTZ3-LNP treatment (48h) for inducing Zta expression in a panel of EBV-positive tumor cell lines, including (FIG. 4a) NPC (C666-1, NNPC76c, C17, NPC43 and NPC-M81), (FIG. 4b) EBVaGC (SNU719, Y CCLE1 and AGS-EBV) and (FIG. 4c) Burkitt lymphoma (P3HR1 and Akata-EBV). SNU719 and C666-1 cells treated with NaB were included as a reference of chemically induced lytic reactivation. FIG. 4d. The percentages of Zta-positive cells in the NPC, EBVaGC and BL cell lines treated with mTZ 3-LNP for 48 h are shown. Data are presented as mean±SD. FIG. 4e, The expression of Zta, its downstream lytic proteins (Rta, BGLF4 and EA-D) and cleaved caspase 3 in YCCEL1, NPC31M81 and C17 cells treated with mTZ3-LNPs were detected by western blotting.

FIG. 5. Effects of mTZ3-LNP treatment on EBV-positive epithelial cancer cells. FIG. 5a, Using RNA sequencing, a few significantly differentially ex pressed genes were detected in EBV-negative HK1 cells treated with mTZ3-LNP. FIG. 5b, Differentially expressed genes identified in SNU719 and C666-1 cells treated with mTZ3-LNP versus those treated with control mRNA-LNP for 48 h. BZLF1 and EBV-encoded transcripts are illustrated in blue and red dots, respectively. FIG. 5c, EBV transcriptome profiles illustrating the induction of multiple EBV lytic genes in SNU719 and C666-1 cells after 48 h of mTZ3-LNP treatment. FIG. 5d, The genomic specificity of the TZ3 TALE transcriptional activator was evaluated in mTZ3-LNP-treated C666-1 cells using ChI P-sequencing with an anti-FLAG antibody. A single peak was illustrated in the promoter region of BZLF1 in the EBV genome from C666-1 cells treated with mTZ3-LNP for 6 h. C666-1 cells treated with control-LNP were included as a control. The experiments were conducted in duplicate. FIG. 5e, The viability of SNU719, C666-1 and HK1 cells treated with mTZ3-LNP alone and a combination of mTZ 3-LNP and GCV for 24, 48, 72 and 96 h was determined. Significant growth inhibition was observed in the EBV-positive SNU719 and C666-1 cells treated with mTZ3-LNP alone and in combination with GCV. Data are presented as mean±SD. ns: not significant; ****: P<0.0001. One-way analysis of variance (ANOVA).

FIG. 6. mTZ3-LNP reactivates EBV lytic gene expression in in vivo EBV-positive tumor models. FIG. 6a, Using immunohistochemical staining, the expression of Zta, EA-D and gp350 was detected in the representative tissue sections of SNU719 tumors from NOD-SCID mouse models at 12, 24 and 48 h after the intravenous administration of mTZ3-LNP. The tumor cells with gp350 ex pression were illustrated by red arrows. Scale bar=50 μm. FIG. 6b, The percentages of tumor cells with Zta, EA-D and gp350 expression in the tumors from the mice were determined at 12, 24 and 48 h post-intravenous administration of mTZ3-LNPs. At least four different representative fields (×200 magnification) obtained from the section of each triplicate were counted. Data are presented as mean±SD. FIG. 6c. The expression of EBV immediate early (BZLF1), early (BMRF1 and BGLF4) and late (BLLF1) lytic gene transcripts in representative FFPE sections of SN U719 tumors from NOD-SCID mouse models at 12 and 48 h post-treatment with mT Z3-LNP was determined using RNAscope RNA in-situ hybridization. Representative tumor cells expressing high and low copy numbers of EBV lytic gene transcripts are indicated by blue and red arrows respectively. Scale bar=50 μm. FIG. 6d. The percentages of tumor cells expressing BZLF1, BGLF4, BMRF1 and BLLF1 mRNAs in the tumors from the mice were determined at 12, 24 and 48 h post treatment with mTZ3-LNP. At least four different representative fields (×200 magnification) obtained from the section of each triplicate were counted. Data are presented as mean±SD.

FIG. 7. In vivo inhibition of EBV-positive epithelial cancers by mTZ 3-LNP-based lytic induction treatment. FIG. 7a, A scheme illustrating the in vivo treatment of EBV-positive EBVaGC (SNU719) and NPC (C666-1, C17, Xeno-76) preclinical xenograft NOD-SCID mouse models with mTZ3-LNP and GCV. FIG. 7b, Tumor volumes were measured throughout the treatment period (n=6-7). Data are presented as mean±SEM. ns: not significant. FIG. 7c, Using EBER in-situ hybridization, EBV-positive tumor cells were detected in representative FFPE sections of residual tumors harvested after treatment. Scale bar=250 μm. Representative images from n=6-7 mice/group are shown. FIG. 7d, The tumor index of each harvested EBV-positive tumor was determined after treatment with mTZ3-LNP alone, combined mTZ3-LNP and GCV, GCV and vehicle controls. Tumor index=tumor volume×percentage of the EBER-positive area. Significant tumor growth inhibition was observed in SNU719, C666-1, C17 and Xeno-76 xenografts treated with mTZ3-LNP alone or combined with GCV (n=6-7/group). Data are presented as mean±SD. ns: not significant; ***: P<0.001; ****: P<0.0001. One-way analysis of variance (ANOVA).

FIG. 8. mRNA-LNP-based lytic induction therapy targets EBV-positive epithelial cancers. Schematic illustration of the synthesis of mTZ3-LNP and its application in lytic induction therapy against EBV-positive epithelial cancers. The LNP-encapsulated modified mRNA mTZ3-LNP encodes the BZLF1-specific TALE transcriptional activator TZ3 to induce the transcription of EBV lytic genes in EBV-positive tumor cells. Treatment induces the lytic cycle to kill EBV-infected cells directly and activate GCV cytotoxicity and bystander killing effects in the tumor. mTZ3-LNP treatment may also activate the host's innate and adaptive immune responses to EBV-positive cancers.

### DEFINITIONS

The term “Epstein-Barr virus” or “EBV” refers to a member of the herp es virus family that is also known as human herpesvirus 4. EBV is classically associated with three malignancies, Burkitt's lymphoma, B-cell lymphoproliferative syndromes, and nasopharyngeal carcinoma. In later studies, EBV has been identified as associated with Hodgkin's disease, T-cell lymphomas, and gastric carcinoma, as well as being the causal agent for infectious mononucleosis. As used herein, the term “EBV-associated cancer” encompasses any pathological conditions or diseases including malignancies where the clonal EBV episomes are detected in the cells of the affected or diseased (e.g., malignant) tissue. Cells taken from pertinent tissues of an EBV-associated disease/pathology (e.g., an EBV-associated cancer) possess at least one copy, possibly more copies, of the EBV genome per cell as verifiable by a polymerase chain reaction (PCR)-based testing methodology.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have desired qualities, e.g., having similar binding properties as the reference nucleic acid, metabolized in a manner similar to naturally occurring nucleotides, and/or encoding a certain amino acid sequence. Unless otherwise indicated, a particular nucleic acid sequence a Iso implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (lea der and trailer) as well as intervening sequences (introns) between individual coding segments (exons).

As used in herein, the terms “identical” or percent “identity,” in the context of de scribing two or more polynucleotide or amino acid sequences, refer to two or more sequences or subsequences that are the same or have a specified percentage of amino acid residues or nucleotides that are the same (for example, a transactivation domain amino acid sequence has at least 80% identity, preferably 85%, 90%, 91%, 92%, 93, 94%, 95%, 96%, 97%, 98%, 99%, or 100% identity, to a reference sequence, e.g., one that is encoded by the 2722-3564 segment of the polynucleotide sequence set forth in SEQ ID NO: 33), when compared and aligned for maximum correspondence over a comparison window, or designated region as measured using one of the following sequence comparison algorithms or by manual alignment and visual inspection.

Such sequences are then said to be “substantially identical.” With regard to polynucleotide sequences, this definition also refers to the complement of a test sequence. Preferably, the identity exists over a region that is at least about 50 amino acids or nucleotides in length, over a region that is 75-100 amino acids or nucleotides in length, over a region that is 200-500 amino acids or nucleotides in length, or over a region that is 500-1000 amino acids or nucleotides in length.

For sequence comparison, typically one sequence acts as a reference sequence, to which test sequences are compared. When using a sequence comparison algorithm, test and reference sequences are entered into a computer, sub sequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window,” as used herein, includes reference to a segment of any one of the number of contiguous positions selected from the group consisting of from about 20 to about 2000, from about 20 to about 1000, 750, 600 or 500, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith &Waterman, Adv. Appl. Math. 2:482 (1981), by the homology alignment algorithm of Needleman &Wunsch, J. Mol. Biol. 48:443 (1970), by the search for similarity method of Pearson &Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215:403-410 and Altschul et al. (1977) Nucleic Acids Res. 25:3389-3402, respectively. Software for performing BLAST analyses is publicly available at the National Center for Biotechnology Information website, ncbi. nlm. nih. gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff &Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nuclear localization signal” or “NLS” refers to an amino acid sequence that is typically a short peptide sequence responsible for direct import of proteins, especially newly synthesized proteins, into the cell nucleus. In genera 1, these sequences contain a high proportion of the basic amino acids lysine and argi nine. Frequently, amino acids such as proline, which disrupt helical domains, are also present. Many NLS sequences are discovered during the analysis of various viral proteins, where the nuclear localizing peptides are covalently linked to the 5′ end of the DNA coding sequence. Well-known viral NLS peptides include those from SV40, HIV, influenza virus, and adenovirus, such as the NLS present in 92-kDa SV40 large T-antigen. Additional exemplary NLS sequences are pro vided in this disclosure, e.g., a 3×NLS encoded by the 91-162 segment of any one of SEQ ID NOs: 31-60.

The term “recombinant” when used with reference, e.g., to a cell, or a nucleic acid, a protein, or a vector, indicates that the cell, nucleic acid, protein or vector, has been modified by the introduction of a heterologous or exogenous nucleic acid or protein or the alteration of a native nucleic acid or protein, or that the cell is deriv ed from a cell so modified. Thus, for example, recombinant cells express genes that are not found within the native (non-recombinant) form of the cell or express native gen es that are otherwise abnormally expressed, under expressed or not expressed at all.

A “promoter” is defined as an array of polynucleotide control sequences that direct transcription of another polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and develop mental conditions. In contrast, an “inducible” promoter is a promoter that rendered active by environmental or developmental regulation and under certain specific environ mental or developmental conditions. The term “operably linked” refers to a functional linkage between a polynucleotide expression control sequence (such as a promoter, or array of transcription factor binding sites) and another polynucleotide sequence (such as a protein-coding sequence), wherein the expression control sequence directs transcription of the second polynucleotide sequence.

An “expression cassette” is a nucleic acid construct, generated recombinantly or synthetically, with a series of specified polynucleotide elements that permit transcription of a particular polynucleotide sequence in a host cell or in an in vitro transcription system (e.g., partially reconstituted cell lysate). An expression cassette may be the entirety or a part of a plasmid, viral genome, or other replicable nucleic acid construct such as episome. Typically, an expression cassette includes a polynucleotide to be transcribed, operably linked to a promoter.

The term “cancer” refers to any of various malignant neoplasms characterized by the proliferation of anaplastic cells that tend to invade surrounding tis sue and metastasize to new anatomic sites within a patient's body. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma, Hodgkin's lymphoma, B-cell lymphoma, or Burkitt's lymphoma), and multiple myeloma.

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process or signal (e.g., a subject's bodyweight, or the blood glucose/cholesterol level, or any measurable symptom or biomarker in a subject, such as an infection rate among subjects by a pathogenic infectious agent or cancer cell proliferation rate/metastatic rate), or any one of the downstream parameters, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “in crease,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or parameter.

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing downstream- and/or side-effects caused by such disease or condition. A preventive measure in this context and its variations do not require 100% elimination of the occurrence of an event; rather, they refer to a suppression or reduction in the likelihood or severity of such occurrence or a delay in such occurrence.

The term “severity” of a disease refers to the level and extent to which a disease pr ogresses to cause detrimental effects on the well-being and health of a patient suffering from the disease, such as short-term and long-term physical, mental, and psychological disability, up to and including death of the patient. Severity of a disease can be reflected in the nature and quantity of the necessary therapeutic and maintenance e measures, the time duration required for patient recovery, the extent of possible recovery, the percentage of patient full recovery, the percentage of patients in need of long-term care, and mortality rate.

A “patient” or “subject” receiving the composition or treatment method of this invent ion is a human, including both adult and juvenile human, of any age, gender, and ethnic background, who may or may not have been diagnosed with any particular disease or disorder (e.g., an EBV-associated cancer) but is at heightened risk of developing such disease and therefore is in need of prophylactic or therapeutic medical intervention (e.g., to reduce or eliminate risk for developing an EB V⁺ cancer). Typically, the patient or subject receiving treatment according to the met hod of this invention to treat or prevent cancer is not otherwise in need of treatment by the same therapeutic agents. For example, if a subject is receiving the nucleic acid composition according to the claimed method, the subject is not suffering from any disease that is known to be treated by the same therapeutic agents. Although a patient may be of any age, in some cases the patient is at least 40, 45, 50, 55, 60, 65, 70, 75, 80, or 85 years of age; in some cases, a patient may be between 40 and 45 years old, or between 50 and 65 years of age, or between 65 and 85 years of age. A “child” subject is one under the age of 18 years, e.g., about 5-17, 9 or 10-17, or 12-17 years old, including an “infant,” who is younger than about 12 months old, e.g., younger than about 10, 8, 6, 4, or 2 months old, whereas an “adult” subject is one who is 18 years or older.

The term “effective amount,” as used herein, refers to an amount that produces intended (e.g., therapeutic or prophylactic) effects for which a composition is administer ed. The effects include the prevention, correction, or inhibition of progression of the symptoms of a particular disease/condition and related complications to any detect able extent, e.g., incidence of an EBV-associated disease, level of severity including mortality rate, one or more of the symptoms of such a disease (e.g., the incidence of an EBV-associated cancer, metastatic rate, and 5- or 10-year survival rat e). The exact amount of an “effective amount” for a particular substance will depend on the purpose of the treatment and is ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “about” when used in reference to a given value denotes a range encompassing ±10% of the value. For example, “about 10” denotes a range of 10+/−1, i.e., 9-11.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of an active agent (e.g., a mRNA LNP formulation of the present invention) effective for treating an EBV-associated disease in a subject is a composition that does not contain any other agents that may have any detectable positive or negative effect on the same target process (e.g., suppress ion of an EBV-associated disease) or that may increase or decrease to any measurable extent of the disease severity among the recipients.

As used herein, the term “particle” refers to a structured entity formed by molecules or molecule complexes, which may be a micro- or nano-sized structure, such as a micro- or nano-sized compact structure dispersed in a medium. For instance, a “particle” may be a nucleic acid-encapsulated particle such as a particle containing DNA, RNA, or a mixture thereof. Electrostatic interactions between positively charged molecules (such as polymers and lipids) and negatively charged nucleic acid are involved in particle formation. This results in complexation and spontaneous formation of nucleic acid particles. Depending on the size, such a nucleic acid-containing particle may be a nanoparticle. As used in the present disclosure, a “nano particle” is a particle having an average diameter suitable for parenteral administration, e.g., in the nanometer range. A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles of this invention include lipid nanoparticle (LNP)-based and lipoplex (LPX)-based formulations.

### DETAILED DESCRIPTION OF THE INVENTION

### I. Introduction

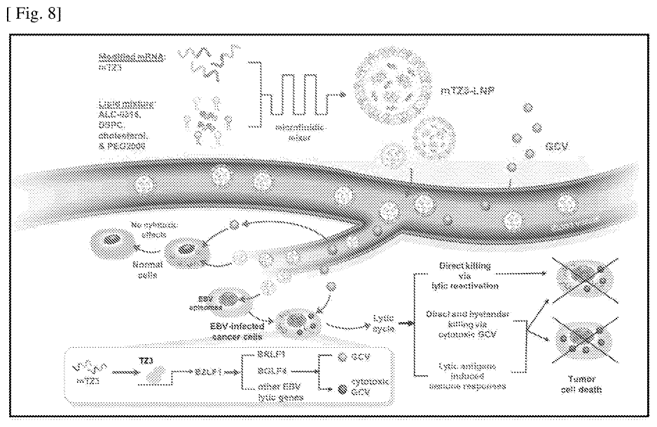

This invention describes a novel method for treating cancers that are associated with latent viral infection, such as EBV-associated cancers, where the cancer cells harbor EBV episomes, by way of inducing the latent virus into its lytic cycle and therefore destroying the cancer cells. For instance, in order to induce latent EBV into the lytic cycle, one or more of the immediate-early (IE) proteins such as BZLF1 and BRLF1 must be expressed, which in turn activate the transcription of early and late the proteins to further drive the lytic cycle. The present invention resides in the use of a synthetic mRNA encoding a fusion protein, which comprises a DNA binding domain linked to a transcriptional activation domain and is therefore able to specifically activate the transcription of EBV-encoded lytic genes such as BZLF1, BRLF1, and BGLF4, to effectively trigger EBV lytic gene expression in EBV-latent infected cancer cells and lead to the lysis of these cells. The synthetic mRNA molecule, preferably modified, can be packaged into lipid nanoparticles (LNPs) or other suitable formulations for delivery to the EBV-associated cancer cells, see FIG. 8.

### II. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in kilobases (kb), base pairs (bp) or nucleotides (nt). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage &Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange HPLC as described in Pearson &Reanier, J. Chrom. 255:137-149 (1983).

The sequence of a gene of interest, a polynucleotide sequence encoding a recombinant polypeptide of interest, and a synthetic oligonucleotide or polynucleotide sequence can be verified after cloning or subcloning using, e.g., the chain termination method for sequencing double-stranded templates of Wallace et a 1., Gene 16:21-26 (1981).

### III. Synthesis and Modification of RNA

The RNA molecule of this invention is designed to encode a fusion protein recombinantly expressed by a host's cells upon receiving the mRNA transcript, which fusion protein specifically targets or binds the promoter sequence of at least one of the EBV IE and E proteins such as BZLF1, BRLF1, and BGLF4 and activates the transcription of the IE and E protein(s) so as to induce the lytic cycle of EBV in cells that have been infected with EBV and remain EBV⁺ (e.g., retaining at least a portion of the EBV genomic sequence in exosomes). Thus, the mRNA of this invention includes the following segments: the first is a coding sequence for a nuclear localization signal, which directs the newly synthesized fusion protein to enter the host cell nucleus to activate transcription of the EBV IE or E protein(s). An exemplary 3×NLS can be found in the 91-162 segment of any one of SEQ ID NOs: 31-60 (underlined with dotted lines in Tables 2-4). The second segment is a coding sequence for a transcription activator-like effector (TALE) domain, which ensures the fusion protein to specifically recognize and bind a pre-selected portion of the promoter sequence for the BZLF1, BRLF1, or BGLF4 protein. TALE are initially derived from naturally occurring proteins secreted by plant pathogenic bacteria Xanthomonads. The recent illustration of the TAL-DNA code now allows one to design a TALE sequence as the DNA-binding domain of an artificial transcription-activating protein for the purpose of targeting the protein to bind any known promoter sequence for a specific gene (e.g., any pre-selected portion of the promoter directing transcription of the EBV IE protein BZLF1 or BRLF1, or E protein BGLF4) in order to activate the transcription of this specific gene. The bold portion of any one of SEQ ID NOs: 31-60 provides an exemplary TALE coding sequence targeting the promoter of the BZLF1, BRLF1, or BGLF4 gene. The third segment is a coding sequence for a transactivation domain, which is capable of initiating the transcription of a gene and is typically taken from a known, naturally-occurring transcription factor, e.g., p53 and p65. The underlined portion of any one of SEQ ID NOs: 31-60 provides an exemplary coding sequence for a transactivation domain. In addition, the fusion protein encoded by the mRNA of this invention may optionally further include one or more epitope tags, typically located at the N- or C-terminus or both of the fusion protein. An example of such an epitope tag is the FLAG tag, e.g., encoded by the 1-69 segment of any one of SEQ ID NOs: 31-60. These coding sequence elements may be selected to form combinations in addition to those shown by SEQ ID NOs: 31-60 (especially TALE-transactivation domain combinations within each group of SEQ ID NOs: 31-40; SEQ ID NOs: 41-50; or SEQ ID NOs: 51-60) to yield further fusion proteins having the utility of activating the lytic cycle of EBV, thus leading to the lysis of EBV-positive cells such as EBV-positive cancer cells.

Polynucleotide sequences including RNA or any derivatives or modified versions thereof may be chemically synthesized according to methods known in the pertinent technical field. An RNA molecule can be modified by substitution with one or more nucleotide analogs and/or at the base moiety, sugar moiety, or phosphate backbone, for example, to improve stability of the molecule, hybridization or binding capability, or bioavailability, etc. The polynucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; WO 88/09810) or the blood-brain barrier (se e, e.g., WO 89/10134), hybridization-triggered cleavage agents (See, e.g., Krol et al., 1988, BioTechniques 6:958-976) or intercalating agents (see, e.g., Zon, 1988, Pharm. Res. 5:539-549). To this end, the RNA molecule can be conjugated to another molecule for purposes such as tissue/cell targeting, stability, bioavailability, and the like.

The RNA molecules of this invention may be synthesized by standard methods known in the art, e.g., by use of an automated polynucleotide synthesizer (such as are commercially available from Biosearch, Applied Biosystems, etc.). As examples, phosphorothioate polynucleotides may be synthesized by the meth od of Stein et al. (1988, Nucl. Acids Res. 16:3209), methylphosphonate polynucleotides can be prepared by use of controlled pore glass polymer supports (Sarin et al., 1988, Proc. Natl. Acad. Sci. U.S.A. 85:7448-7451) etc.

### A. In Vitro Synthesis of RNA

In addition to chemical synthetic methods, production of RNA molecules of the present invention can be carried out by recombinant nucleic acid techniques. To obtain a high level of an RNA transcript of a nucleic acid encoding a desired polypeptide, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Samb rook and Russell, supra, and Ausubel et al., supra.

For instance, the RNA molecules of this invention are in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a polynucleotide sequence encoding a recombinant protein of interest and introducing it into an appropriate vector for in vitro transcription.

In some embodiments, RNA according to the present disclosure comprises a 5′-UTR and/or a 3′-UTR. The term “untranslated region” or “UTR” relates to a region in a DNA molecule that is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start cod on of a protein-encoding region. A 5′-UTR is downstream of the 5′-cap (if present), e.g., directly adjacent to the 5′-cap. A 3′-UTR, if present, is located at the 3′end, downstream of the termination codon of a protein-encoding region, but the term “3′-UTR” does preferably not include the poly (A) sequence. Thus, the 3′-UTR is upstream of the poly (A) sequence (if present), e.g., directly adjacent to the poly (A) sequence.

In some embodiments, the RNA of the present invention comprises a 3′-poly (A) sequence. As used herein, the term “poly (A) sequence” or “poly-A tail” refers to a string of uninterrupted or interrupted adenylate residues located at the 3′-end of an RNA molecule. The RNA molecules of this invention can have a poly (A) sequence attached to the free 3′-end of the RNA by a template-independent RNA polymerase after transcription or a poly (A) sequence encoded by DNA and transcribed by a template-dependent RNA polymerase. It has been demonstrated that a poly (A) sequence of about 120 A nucleotides has a beneficial influence on the levels of RNA in transfected eukaryotic cells, as well as on the levels of protein that is translated from an open reading frame that is present upstream (5′) of the poly (A) sequence (Holtkamp et al., 2006, Blood, vol. 108, pp. 4009-4017). For the present invention, the poly (A) sequence may be of any length. In some embodiments, a poly (A) sequence comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly (A) sequence, typically at least 75%, at least 80%, at least 85%, at least 90%, at least 95%, at least 96%, at least 97%, at least 98%, or at least 99% by number of nucleotides in the poly (A) sequence are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly (A) sequence, i.e., 100% by number of nucleotides in the poly (A) sequence, are A nucleotides. In some embodiments, a poly (A) sequence is attached during RNA transcription, e.g., during preparation of in vitro transcribed RNA, based on a DNA template comprising repeated dT nucleotides (deoxythymidylate) in the strand complementary to the coding strand. The DNA sequence encoding a poly (A) sequence (coding strand) is referred to as poly (A) cassette.

In some embodiments, the RNA of the present invention comprise one or more modified nucleosides as described herein and by methods known in the art. For example, the RNA may include a modified nucleoside in place of at least one (e.g., every) uridine.

When an RNA transcript is produced in an in vitro system in satisfying quantity, it may be purified following the standard nucleic acid purification procedure including size differential filtration and column chromatography. The identity of the RNA molecule may be further verified by methods such as nucleic acid sequence analysis and mass spectrometry.

### B. Modified Nucleotides, Nucleosides and Polynucleotides

When in reference to a nucleotide, nucleoside or polynucleotide (such as the nucleic acids of the invention, e.g., mRNA molecule), the terms “modification” and “modified” describe modification with respect to A, G, U and C ribonucleotides. Generally, the se terms are not intended to refer to the ribonucleotide modifications in naturally occurring 5′-terminal mRNA cap moieties.

The modifications may be various distinct modifications. In some embodiments, where the nucleic acid is an mRNA, the coding region, the flanking regions and/or the terminal regions may contain one, two, or more (optionally different) nucleoside or nucleotide modifications.

The polynucleotides can include any useful modification, such as to the sugar, the nucleobase, or the inter-nucleoside linkage (e.g., to a linking phosphate/toa phosphodiester linkage/to the phosphodiester backbone). For example, the major groove of a polynucleotide, or the major groove face of a nucleobase may comprise one or more modifications. One or more atoms of a pyrimidine nucleobase (e.g., on the major groove face) may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), e.g., the substitution of the 2′OH of the ribofuranysyl ring to 2′H, threose nucleic acids (TNAs), glycol nucleic acids (GNAs), peptide nucleic acids (PNAs), locked nucleic acids (LNAs) or hybrids thereof).

The polynucleotides of the invention do not substantially induce an innate immune response of a cell into which the polynucleotide (e.g., mRNA) is introduced. Features of an induced innate immune response include 1) increased expression of pro-inflammatory cytokines; 2) activation of intracellular PRRs (RIG-I, MDA5, etc.; and/or 3) termination or reduction in protein translation.

In certain embodiments, it may desirable for a modified nucleic acid molecule introduced into the cell to be degraded intracellularly. For example, degradation of a modified nucleic acid molecule may be preferable if precise timing of protein production is desired. Thus, in some embodiments, the invention provides a modified nucleic acid molecule containing a degradation domain, which is capable of being acted on in a directed manner within a cell. In other embodiments, a modified polynucleotide introduced to a cell may exhibit reduced degradation in the cell, as compared to an unmodified polynucleotide. In another aspect, the present disclosure provides polynucleotides comprising a nucleoside or nucleotide that can disrupt the binding of a major groove interacting, e.g., binding, partner with the polynucleotide (e.g., where the modified nucleotide has decreased binding affinity to major groove interacting partner, as compared to an unmodified nucleotide).

The modified nucleosides and nucleotides (e.g., building block molecules), which may be incorporated into a polynucleotide (e.g., RNA or mRNA, as described herein), can be modified on the sugar of the ribonucleic acid.

For example, the 2′hydroxyl group (OH) can be modified or replaced with a number of different substituents. Exemplary substitutions at the 2′-position include, but are not limited to, H, halo, optionally substituted C1-6 alkyl; optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein); “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid, and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The present disclosure provides for modified nucleosides and nucleotides. As described herein “nucleoside” is defined as a compound containing a sugar molecule (e.g., a pentose or ribose) or derivative thereof in combination with an organic base (e.g., a purine or pyrimidine) or a derivative thereof (also referred to here in as “nucleobase”). As described herein, “nucleotide” is defined as a nucleoside including a phosphate group. In some embodiments, the nucleosides and nucleotides described herein are generally chemically modified on the major groove f ace. Exemplary non-limiting modifications include an amino group, a thiol group, an alkyl group, a halo group, or any described herein. The modified nucleo tides may by synthesized by any useful method, as described herein (e.g., chemically, enzymatically, or recombinantly to include one or more modified or non-natural nucleosides).

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. These nucleobases can be modified or wholly replaced to provide polynucleotide molecules having enhanced properties, e.g., resistance to nucleases, stability, and these properties may manifest through disruption of the binding of a major groove binding partner. For example, the nucleosides and nucleotides described can be chemically modified on the major groove face. In some embodiments, the major groove chemical modifications can include an amino group, a thiol group, an alkyl group, or a halo group.

In some embodiments, the modified nucleobase is a modified uracil. Exemplary nucleobases and nucleosides having a modified uracil include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridineor 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1-methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5, 6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropyl) pseudouridine, 5-(isopentenylaminomethyl) uridine, 5-(isopentenylaminomethyl)-2-thio-uridine, a-thio-uridine, 2′-O-methyl-uridine, 5, 2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3, 2′-O-dimethyl-uridine, and 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine.

In some embodiments, the modified nucleobase is a modified cytosine.

Exemplary nucleobases and nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine (e.g., 5-iodo-cytidine), 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, a-thio-cytidine, 2′-O-methyl-cytidine, 5, 2′-O-dimethyl-cytidine, N4-acetyl-2′-O-methyl-cytidine, N4, 2′-O-dimethyl-cytidine, 5-formyl-2′-O-methyl-cytidine, N4, N4, 2′-O-trimethyl-cytidine, 1-thio-cytidine, 2′-F-ara-cytidine, 2′-F-cytidine, and 2′-OH-ara-cytidine.

In some embodiments, the modified nucleobase is a modified adenine.

Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2, 6-diaminopurine, 7-deaza-8-aza-2, 6-diaminopurine, 1-methyl-adenosine (m′A), 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine. N6, 2′-O-dimethyl-adenosine, N6, N6, 2′-O-trimethyl-adenosine, 1, 2′-O-dimethyl-adenosine, 2′-O-ribosyladenosine, 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine.

Exemplary nucleobases and nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-methyl-6-thio-guanosine, N2-methyl-6-thio-guanosine, N2, N2-dimethyl-6-thio-guanosine, α-thio-guanosine, 2′-O-methyl-guanosine, N2-methyl-2′-O-methyl-guanosine, N2, N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 2′-O-ribosylguanosine, 1-thio-guanosine, 06-methyl-guanosine, 2′-F-ara-guanosine, and 2′-F-guanosine.

In some embodiments, the nucleotide can be modified on the major groove face. For example, such modifications include replacing hydrogen on C-5 of uracil or cytosine with alkyl (e.g., methyl) or halo.

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In another embodiment, the nucleobase can also include, for example, naturally-occurring and synthetic derivatives of a base, including pyrazolo[3, 4-d] pyrimidines, 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo [1,5-a] 1, 3, 5 triazinones, 9-deazapurines, imidazo[4,5-d]pyrazines, thiazolo [4,5-d]pyrimidines, pyrazin-2-ones, 1, 2, 4-triazine, pyridazine; and 1, 3, 5 triazine. When the nucleotides are depicted using the shorthand A, G, C, T or U, each letter refers to the representative base and/or derivatives thereof, e.g., A includes adenine or adenine analogs, e.g., 7-deaza adenine).

Upon completion of synthesis and isolation/purification of an RNA molecule, it can be tested to confirm its ability to cause death or suppress proliferation of EBV⁺ cells by activating EBV lytic cycle in such cells. For instance, a cell-based assay is performed in vitro by contacting EBV-positive cells with an adequate amount of a test RNA molecule to observe any inhibitory effect on the proliferation of the EBV-positive cells. When an inhibitory effect (e.g., at least 25%, 50%, 80%, 90% or more inhibition in proliferation or viability of treated EBV-positive cells in comparison with untreated EBV-positive cells of the same type) is detected, the RNA molecule is deemed to be effective for use in the method of this invention to treat an EBV-associated pathology (e.g., an EBV⁺ cancer). For further verification of its activity, the RNA molecule may be optionally subject to testing in an animal model, for example, by administering the RNA molecule in an adequate quantity (e.g., by injection) into immunocompromised animals bearing xenograft EBV⁺ tumors to observe an inhibitory effect (e.g., at least 25%, 50%, 80%, 90% or more inhibition) on tumor growth and/or metastasis in comparison to untreated control animals for confirmation of activity.

### IV. Pharmaceutical Compositions and Administration

The present invention provides pharmaceutical compositions comprising an effective amount of RNA encoding a recombinant protein capable of activating EBV immediate-early (IE) proteins (such as BZLF1 and BRLF1) expression and therefore inducing EBV lytic cycle for the purpose of treating an EBV-associated disease, especially EBV⁺ cancers, in a person suffering from such a disease. Pharmaceutical compositions of the invention are suitable for use in a variety of drug delivery systems. Suitable formulations for use in the present invention are found, e.g., in Remington's Pharmaceutical Sciences, Mack Publishing Company, Philadelphia, PA, 17th ed. (1985). For a brief review of methods for drug delivery, see, Langer, Science 249:1527-1533 (1990).

The pharmaceutical compositions of the present invention can be administered by various routes, e.g., systemic administration via oral ingestion or injection (e.g., intravenous, intramuscular, or subcutaneous injection) as well as local delivery such as by intratumoral, intracranial, or intraperitoneal injection or by direct (e.g., topical) application or by using an appropriate suppository. One preferred route of administering the pharmaceutical compositions is intravenous administration at daily doses of about 1 to about 1000 μg, about 5 to about 500 μg, about 10 to about 250 μg, about 20 to about 100 μg, or about 25 to about 50 μg of the RNA of this invention. Additionally, the composition may be formulated in a daily, weekly, or monthly dosage format for administration to the subject. The appropriate dose may be administered in a single, one-time daily dose or as divided doses presented at ap propriate intervals, for example one dose every two, three, four, five, six, or more months such as every 12 months.

For preparing pharmaceutical compositions containing the RNA molecules of this invention, one or more inert and pharmaceutically acceptable carriers are used. Depending on the means of administration, the pharmaceutical carrier can be either solid or liquid. Solid form preparations include, for example, powders, creams/pastes, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

Powders and other versions of solid compositions contain an adequate amount of the active ingredient(s) (e.g., the mRNA of the present invention, option ally with another anti-cancer therapeutic agent) along with one or more carriers. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid pharmaceutical compositions include, for example, solutions suitable for oral administration or local delivery, suspensions, and emulsions suitable for oral administration. Sterile water solutions of the active component (e.g., the mRNA of the present invention, optionally with another anti-cancer agent) or sterile solutions of the active component in solvents comprising water, buffered water, saline, PBS, ethanol, or propylene glycol are examples of liquid or semi-liquid compositions suitable for oral administration or local delivery such as by topical application or rectal suppository. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., the RNA of the present invention, optionally further in combination with one or more anti-cancer therapeutic agent) in the desired solvent system, and then passing the resulting solution through a membrane filter to sterilize it or, alternatively, by dissolving the sterile active component in a previously sterilized solvent un der sterile conditions. The resulting aqueous solutions may be packaged for use as is, or lyophilized, the lyophilized preparation being combined with a sterile aqueous carrier prior to administration. The pH of the preparations is typically between about 3 and about 11, for example, from about 5 to about 9, or from about 7 to about 8. Single or multiple administrations of the compositions can be carried out with dose levels and pattern being selected by the treating physician. In any event, the pharmaceutical formulations should provide a quantity of an active agent (e.g., an mRNA molecule encoding a recombinant protein capable of activating EBV IE protein expression as described herein) sufficient to trigger the EBV lytic cycle.

In some embodiments, the composition comprising the RNA of this invention is formulated as a composition of nucleic acid particles, especially in the form of lipid nanoparticles (LNP) comprising the RNA. One or more types of lipid, as well as other ingredients, may be used in the formulation. For example, the LNP may comprise a cationic lipid, a neutral lipid, a steroid, a polymer conjugated lipid, and the RNA. In some cases, the LNP may further comprise at least one lipid or lipid-like material other than a cationic or cationically ionizable lipid or lipid-like material, at least one polymer other than a cationic polymer, or a mixture there of. In some embodiments, the ratio of mRNA to total lipid (N/P) is between 5 and 10 such as about 6 or about 7. Nucleic acid particles of this invention may have an average diameter ranging from about 30 nm to about 1000 nm, from about 50 nm to about 800 nm, from about 70 nm to about 600 nm, from about 90 nm to about 400 nm, or from about 100 nm to about 300 nm. The nucleic acid particles may exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

The preferred mode of administration of such RNA-encapsulated LNP compositions is intertumoral or intravenous administration, more preferably in aqueous cryoprotectant buffer for intertumoral or intravenous administration. The composition is often a preservative-free, sterile dispersion of RNA formulated in lipid nanoparticles (LNP) in aqueous cryoprotectant buffer for intertumoral or intravenous administration.

### V. Additional Therapeutic Agents

Additional known therapeutic agent or agents with anti-cancer efficacy may be used in combination with an mRNA as described herein during the practice of the present invention for the purpose of treating EBV-associated cancer by way of inducing EBV lytic cycle. In such applications, one or more of these previously known effective anti-cancer therapeutic agents can be administered to patients concurrently with an effective amount of the RNA formulation either together in a single composition or separately in two or more different compositions. They may be used in combination with the active agent of the present invention (such as the mRNA lipid nanoparticles) to suppress cancer growth, inhibit cancer metastasis, and facilitate remission from the disease.

For example, various chemotherapeutic agents are known to be effective for use to treat various cancers. As used herein, a “chemotherapeutic agent” encompasses any chemical compound exhibiting suppressive effect against cancer cells, thus useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods disclosed herein also include cytostatic and/or cytotoxic agents.

Exemplary anti-cancer therapeutic agents include alkylating agents such as altretamine, bendamustine, busulfan, carboquone, carmustine, chlorambucil, chlormethine, chlorozotocin, cyclophosphamide, dacarbazine, fotemustine, ifosfamide, lomustine, melphalan, melphalan flufenamide, mitobronitol, nimustine, nitrosoureas, pipobroman, ranimustine, semustine, streptozotocin, temozolomide, thio tepa, treosulfan, triaziquone, triethylenemelamine, trofosfamide, and uramustine; anthracyclines such as aclarubicin, daunorubicin, doxorubicin, epirubicin, idarubicin, mitoxantrone, pirarubicin, valrubicin, and zorubicin; cytoskeletal disruptors (taxanes) such as abraxane, cabazitaxel, docetaxel, larotaxel, paclitaxel, taxotere, and tesetaxel; epothilones such as ixabepilone; histone deacetylase inhibitors such as vorinostat, romidepsin, and inhibitors of topoisomerase I such as belotecan, camptothecin, exatecan, gimatecan, irinotecan, and topotecan; inhibitors of topoisomerase II such as etoposide, teniposide, and tafluposide; kinase inhibitors such as bortezomib, erlotinib, gefitinib, imatinib, vemurafenib, and vismodegib; nucleotide analogs and precursor analogs such as azacitidine, azathioprine, capecitabine, cytarabine, doxifluridine, fluorouracil, gemcitabine, hydroxyurea, mercaptopurine, methotrexate, and tioguanine (formerly thioguanine); peptide antibiotics such as actinomycin and bleomycin; platinum-based agents such as carboplatin, cisplatin, dicycloplatin, oxaliplatin, nedaplatin, and satraplatin; retinoids such as alitretinoin, bexarotene, and tretinoin; and vinca alkaloids and derivatives such as vinblastine, vincristine, vindesine, and vinorelbine.

In one particular embodiment of the present invention, anti-cancer drug ganciclovir (GCV) is used in conjunction with the RNA composition(s) described herein, especially one or more compositions comprising an RNA encoding a recombinant protein targeting to activate the EBV BGLF4 protein (e.g., any one of SEQ ID NOs: 21-30), to enhance and facilitate its anti-cancer effects in suppressing and eliminating EBV-positive cancer cells.

### VI. Kits

The invention also provides kits for treating EBV-associated diseases, especially EB V-associated cancers, according to the method disclosed herein. The kits typically include a plurality of containers, each containing a pharmaceutical composition comprising one or more of the RNA formulations (e.g., RNA LNPs of this invent ion). Optionally, additional container(s) may be included in the kit each providing pharmaceutical composition(s) comprising one or more of known cancer drugs to be administered concurrently with the RNA composition(s) of the present disclosure. The kits may further include informational material providing instructions on how to dispense the pharmaceutical compositions, including description of the ty pe of patients who may be treated (e.g., human patients who have received a diagnosis of any one of such diseases or who have been deemed to have a heightened risk of dev eloping any one of such diseases at a later time and are therefore seeking to treat such diseases or to reduce future risks of developing such diseases), as well as the dosing and administration schedule for distributing the pharmaceutical compositions to the patients.

### EXAMPLES

The following examples are provided by way of illustration only and not by way of limitation. Those of skill in the art will readily recognize a variety of non-critical parameters that could be changed or modified to yield essentially the same or similar results.

The unique virus-cell interaction in Epstein-Barr virus (EBV)-associated malignancies implies targeting the viral latent-lytic switch is a promising therapeutic strategy. However, the lack of specific and efficient therapeutic agents to induce lytic cycle in these cancers is a major challenge facing clinical implementation. We developed a synthetic transcriptional activator that specifically activates endogenous BZLF1 and efficiently induces lytic reactivation in EBV-positive cancer c ells. A lipid nanoparticle encapsulating nucleoside-modified mRNA encoding a BZLF1-specific transcriptional activator (mTZ3-LNP) was synthesized for EBV-targeted therapy. Compared with conventional chemical inducers, mTZ3-LNP more efficiently activated EBV lytic gene expression in EBV-associated epithelial cancers. We demonstrated the potency and safety of treatment with mTZ3-LNP to suppress tumor growth in EBV-positive tumor xenograft models. The combination of mTZ3-LNP and ganciclovir yielded highly selective cytotoxic effects of mRNA-based lytic induction therapy against EBV-positive tumor cells, indicating the potential of mRNA nanomedicine in the treatment of EBV-associated epithelial cancers.

### INTRODUCTION

Epstein-Barr virus (EBV) is the first cancer-associated virus identified in humans and it affects more than 90% of the global population. While EBV carriers remain mostly asymptomatic lifelong, latent EBV infection contributes to the transformation and progression of a variety of human malignancies, including endemic Burkitt lymphoma (BL), Hodgkin's lymphoma, natura 1 killer (NK)-/T-cell lymphoma, post-transplant lymphoma, nasopharyngeal carcinoma (NPC) and a subset of gastric cancers (GC). These cancers have a global incidence rate of more than 265,000 persons/year and a mortality rate of more than 164,000 persons/year (1, 2). All EBV-associated tumors share unique features including the continued presence of multiple viral genomes (up to 100 genomes/cell) and a restricted latency program. In these malignancies, EBV maintains episomal genomes and expresses multiple latent genes to modulate cancer hallmarks. The presence of viral episomal genomes in all EBV-associated cancer cells serves as a tumor-specific target for the development of efficient therapeutic strategies against these cancers (2, 3).

Switching the EBV-infected cells from latent to lytic cycle can induce growth arrest, promote apoptosis and cause lytic rupture of the cells, and thereby being an attractive approach to curing EBV-associated malignancies (4-9). When a latent EBV is induced into the lytic cycle, the immediate-early (IE) proteins, Zta and Rta encoded by BZLF1 and BRLF1, respectively, must be expressed. These proteins then activate the transcription of a panel of early (e.g., EA-D, BGLF4) and late (e.g., VCA, gp350) proteins to facilitate the lytic replication of EBV genomes and production of infective virions (7-9). Reactivation of EBV from latency is dependent on the expression of the viral Zta protein. Ectopic BZLF1 expression alone can trigger the switch from the latent to the lytic stage and drive EBV lytic cycle completion in EBV-infected cells. Therefore, cytolytic virus activation (CLVA), in which chemical lytic inducers (e.g., gemcitabine, valproic acid, sodium butyrate and other histone deacetylase inhibitors) are used to trigger EBV to enter the lytic phase, has been developed as a therapeutic strategy to specifically target EBV-associated cancers. In this type of lytic induction therapy, antiviral ganciclovir (GCV) is co-administrated with the chemical inducer to patients to mediate specific cell killing and prevent virus production in EBV-infected cells. GCV is non-cytotoxic to EBV-positive tumors with restricted viral latency. However, BGLF4, an EBV-encoded serine/threonine kinase is expressed during lytic reactivation and can convert the non-cytotoxic GCV to an active, cytotoxic form via phosphorylation. This BGLF4-converted cytotoxic GCV or phosphorylated GCV rapidly kills cancer cells and has been evaluated in clinical trials of oncolytic therapy against EBV-associated cancers (3, 7-9). In addition to mediating the direct killing of EBV-positive tumor cells after lytic reactivation, the phosphorylated GCV can be transferred to adjacent cells, leading to a “bystander killing” effect. Importantly, phosphorylated GCV can inhibit EBV encoded DNA polymerase, interrupting the production of infective virions and preventing dissemination of virus during lytic induction therapy (7-11).

CLVA treatment has been evaluated in phase-I/II clinical trials involving patients with recurrent NPC and has elicited a clinical response in some patients (10, 11). However, the efficiency of chemical activators for inducing lytic reactivation in EBV-associated tumors often has been low and variable. These activators also have low specificity for EBV activation and a broad-spectrum of cytotoxicity, meaning that the treated cells probably die from the toxic effect of the chemicals before viral lytic reactivation is induced. Recent studies have revealed that chemical activator-induced EBV lytic reactivation is cell context-specific. The efficiency of chemical activator-based lytic induction therapy depends upon a variety of acquired epigenetic changes and cellular transcription factors in the tumor cells (7-9). In native EBV-associated gastric cancer (EBVaGC) and NPC cell lines (e.g., SNU719, C666-1, NPC43 and C17), weak or no lytic gene expression was detected after treatment with various chemical inducers. Only small proportions of tumor cells with lytic gene expression was observed, even in tumors that responded to treatment with chemical inducers. None of the reported chemical inducers could universally reactivate the lytic cycle in all native EBV-positive epithelial cancer cell lines.

In this study, we explored whether a synthetic BZLF1-specific transcriptional activator could bypass the restrictions imposed by various cellular factors on EBV reactivation and improve the specificity of lytic induction therapy. By exploiting the CRISPR-Casilio activator system, we demonstrated the feasibility of using an artificial activator to reactivate EBV lytic genes and the cytotoxic effect of this activator in NPC and EBVaGC cells (12). A potential artificial transcriptional activator-binding sequence in BZLF1 promoter was also identified using a panel of designed single-guide RNAs (sgRNAs) in this system. The CRISPR-Casilio system, a hybrid system combining CRISPR/dCas9 and a Pumilio RNA-binding domain-fused effector, consists a dcas9 protein, a Pumilio/fem-3 mRNA binding factor (PUF)-p65HSF1 activator module and a sgRNA appended with five PUF-binding sites type a (PBSa) encoded by three lentiviral constructs (12). The complexity of the system potentially poses delivery challenges that reduce the efficiency of in vivo activation of BZLF1 in EBV-positive epithelial cancers and limits its clinical applications. In contrast to the CRISPR-Casilio system, the transcription activator-like effector (TALE)-based activator system encodes only a synthetic protein with a designed DNA-binding domain fused to the p65HSF1 transactivator. The simplicity of the TALE-based transcriptional activator system implies its usefulness in the development of EBV lytic induction therapy for clinical applications (13). To achieve successful in vivo therapeutic delivery of BZLF1-specific artificial transcriptional activation system, we synthesized a TALE-based transcriptional activator for the efficient induction of EBV lytic reactivation based on the sgRNA-binding sequence identified in the BZLF1 promoter. Nucleoside-modified mRNAs encoding this BZLF1-specific TALE-transcriptional activator were then synthesized and encapsulated in formulated lipid nanoparticles (LNPs) for efficient delivery to EBV-associated epithelial cancers and induction of the EBV immediate early lytic gene BZLF1 transcription, subsequently switching toward the lytic cycle in tumor cells. In both in vitro and in vivo EBV-positive tumor models, the LNP-encapsulated mRNA encoding a BZLF1-specific TALE-transcriptional activator induced the EBV lytic cycle with high efficiency and was universally applicable to all EBV-positive epithelial cancers. The potent and specific cytotoxic effect of this EBV-targeted mRNA drug in both in vitro and in vivo tumor models imply its potential as a promising nanomedicine therapy for EBV-associated malignancies.

### Results

### CRISPR-Casilio Activator System Induces EBV Lytic Reactivation

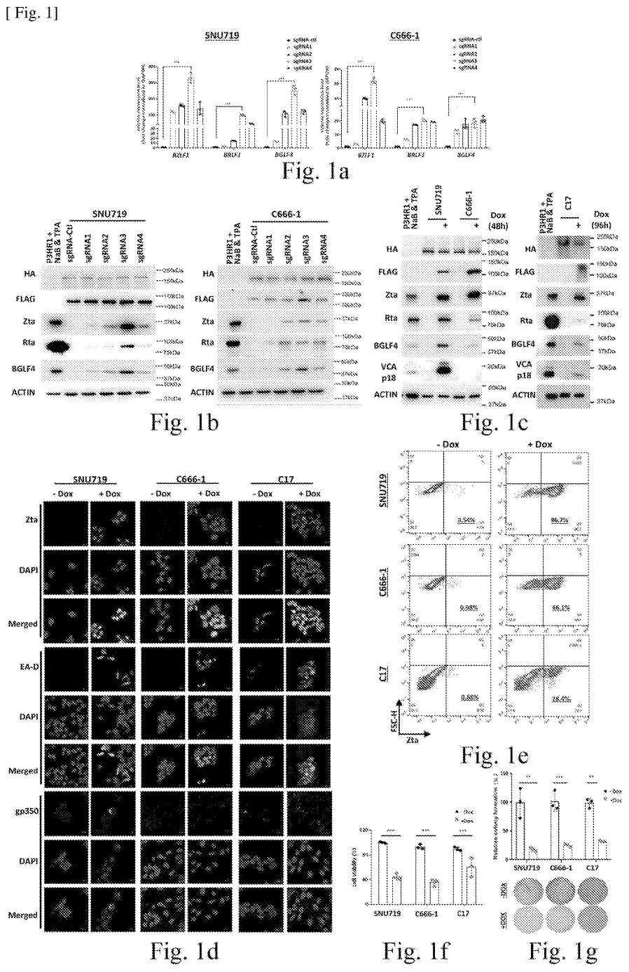

To reactivate the EBV lytic cycle in EBVaGC and NPC cells, we first exploited the efficient CRISPR-based Casilio activator system to induce the endogenous expression of the EBV-encoded immediate early lytic gene BZLF1. The Casilio activator system consists of a dCas9 protein, a PUFa-p65HSF1 activator module and a sgRNA appended with five copies of PBSa (12). It was specifically designed for recruitment of multiple PUF-p65HSF1 activators to enable the potent transcriptional activation of target genes. Given the high feasibility of sgRNA synthesis, this system can be used to screen multiple target sequences for efficient transcriptional activation. Here, the EBVaGC cell line SNU719 and the NPC cell line C666-1 were co-transduced with lentiviral vectors encoding HA-dCas9-EGFP, 3xFLAG-PUFa-p65HSF and a panel of sgRNAs-5×PBSa that can bind to the BZLF1 promoter (sgRNA1, sgRNA2, sgRNA3, sgRNA4) (Tables 8-9). Among the sgRNAs used for the co-transfection of SNU719 and C666-1 cells, sgRNA3 induced the highest expression of BZLF1 and Zta as well as another immediate early lytic protein, Rta, and the early lytic protein, BGLF4 (FIG. 1a-b). The high efficiency of sgRNA3 in inducing EBV lytic reactivation was further demonstrated in SNU719, C666-1 and C17 cells engineered using an inducible Casilio (iCasilio) activator system. The iCasilo system was delivered by transduction with lentiviral vectors constitutively expressing sgRNA3, HA-dCas9-2A-EGFP and a piggyBac transposon containing the Tet-On 3xFLAG-PUFa-p65HSF transactivator (Table 2). The treatment of stably transfected SNU719 and C17 cells with doxycycline (Dox) resulted in transactivator induction and the expression of Zta, Rta, and downstream lytic proteins (e.g., BGLF4, EA-D, VCA and gp350) in these EBV-positive epithelial cancer cells (FIG. 1c-e). The supernatants of the treated cells were collected, and the presence of infectious EBV virions was demonstrated by the successful infection of Akata^{EBV-negative} cells with EBV. Transcripts of EBV-encoded genes were detected in the re-infected Akata cells. The findings confirmed the endogenous activation of BZLF1 induced a complete lytic cycle and the production of infective EBV virions in SNU719 and C17 cells. As reported previously, we observed the induction of an abortive early lytic cycle pattern in C666-1 cells (6, 14). In stably transfected C666-1 cells treated with Dox, no late lytic proteins (VCA and gp350) were detected, whereas immediate early (Zta, Rta) and early (BGLF4) lytic proteins were induced (FIG. 1c-e). The endogenous activation of BZLF1 showed significant in vitro cytolytic effects in EBVaGC and NPC cells. The Casilio-mediated artificial activation of endogenous BZLF1 expression significantly inhibited the cell viability and colony formation ability of EBV-positive cancer cells (FIG. 1f-g). Strikingly, the potent cytotoxic effects of artificial activation of BZLF1 were also demonstrated in vivo in nude mouse models implanted with iCasilio-engineered EBV-positive epithelial cancer cells. In mouse models of EBV-positive epithelial cancer, daily Dox treatment alone or in combination with GCV had a dramatic inhibitory effect on tumor formation. These findings demonstrated that the artificial activation of endogenous BZLF1 expression could efficiently reactivate the lytic cycle and induces a cytolytic effect in EBV-positive epithelial cancers.

### Development of a TALE Activator for the Artificial Activation of BZLF1

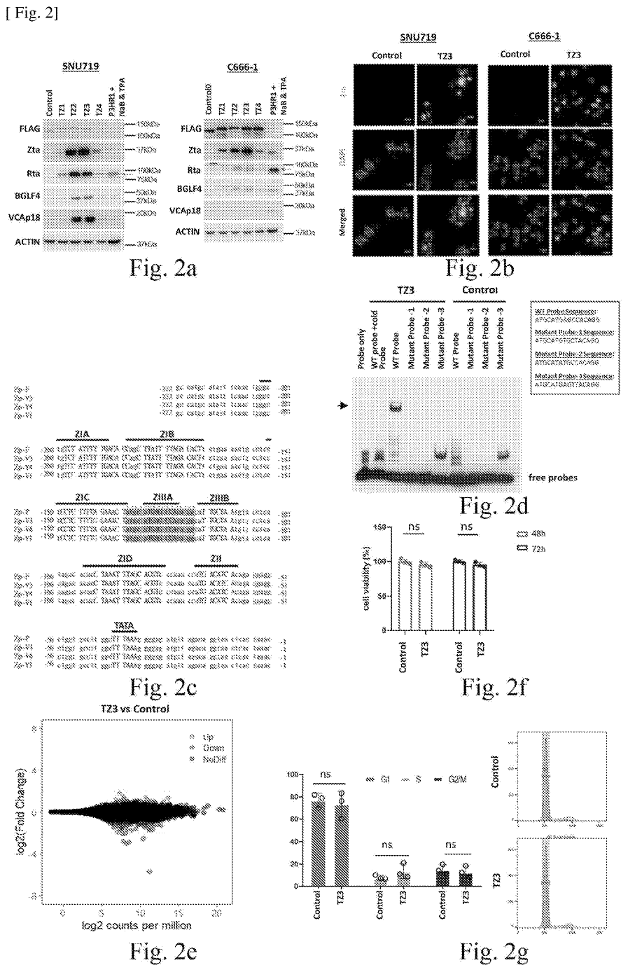

Using the well-established Casilio activation system with designed sgRNAs, we identified a target sequence in the BZLF1 promoter for artificial activation, in support of the effort to develop a synthetic transcriptional activator for efficient EBV lytic induction therapy. However, it would be challenging to effectively deliver the complex Casilio activator system containing three complex and large constructs for the targeting of EBV-positive tumors in patients. We assembled a single construct encoding a precise and efficient TALE transcriptional activator that comprised only a central repeat domain to enable DNA recognition, nuclear localization signals (NLS), and a transcriptional activation domain to artificially activate BZLF1 transcription. We designed and constructed a TALE activator plasmid, TZ3, that would specifically target a predicted binding sequence in the BZLF1 promoter to activate endogenous Zta expression in EBV-positive tumor cells (Tables 8-10). The constructed TALE transcriptional activator has an open reading frame of approximately 3.6 kb that encoded a FLAG tag and the designed TALE DNA-binding and NLS domains, fused to the p65HSF1 transactivator. In addition to TZ3, we also constructed TALE transcriptional activators that targeted regions overlapping with the binding sequences of sgRNA1, sgRNA2, and sgRNA4 (TZ1, TZ2, and TZ4, respectively) to determine the concordance of Casilio- and TALE-based artificial transcriptional activation systems. As shown in FIG. 2a-b, Zta expression was induced in SNU719 and C666-1 cells transiently transfected with BZLF-targeted TALE plasmids, but not in cells transfected with a control vector lacking the DNA-binding domain. In addition to Zta, the expression of downstream immediately early (Rta), early (BGLF4) and late (VCA) lytic proteins was detected in the EBV-positive SNU719 cells transfected with TZ3. Furthermore, TZ3 induced high levels of expression of immediate early (Zta, Rta) and early (BGLF4) lytic proteins in C666-1 cells. These findings confirmed that ectopic expression of the synthetic transcriptional activator TZ3 successfully reactivated the virus lytic cycle in EBV-positive tumor cells. Consistent with the findings obtained when using Casilio activator system with sgRNA3, TZ3 induced the highest level of Zta expression when compared with the other TALEs (TZ1, TZ2 and TZ4).

As shown in FIG. 2c, the TZ3-targeted sequence in the BZLF1 promoter is conserved across all reported EBV variants (15). Using electrophoretic mobility shift assay (EMSA) analysis, we showed the binding of TZ3 activator protein to the predicted BZLF1 promoter sequence in the EBV genome, but not in the mutant probes (FIG. 2d). These findings proved that the synthetic TZ3 protein specifically bound to the BZLF1 promoter in the EBV genome to activate the transcription of this immediate early lytic gene. Furthermore, the transient transfection of HK1 cells, an EBV-negative NPC cell line, with TZ3 did not induce significant changes in the transcriptome or in the cell viability and cell cycle regulation processes (FIG. 2e-g). These findings demonstrated the high specificity of the synthetic TALE-transcriptional activator for targeting EBV-positive tumor cells. Next, TZ3 was used for the further development of TALE-based lytic induction therapy EBV-associated epithelial cancers.

Synthesized nucleoside-modified mRNA encoding TZ3 lytic activator To efficiently activate endogenous BZLF1 expression for clinical application, we synthesized nucleoside-modified mRNAs encoding TALE-based transcriptional activators for in vivo delivery using LNPs. The T7 promoter in the TZ3 construct was used to initiate the in vitro transcription (IVT) of TZ3 mRNA. For in vitro and in vivo studies, nucleoside-modified TZ3 mRNAs with 5′-capping and a 3′-poly-A tail were synthesized and encapsulated by the formulated LNPs containing ALC-0315, 1, 2-distearoyl-sn-glycero-3-phosphocholine (D SPC), cholesterol and 1,2-dimyristoyl-rac-glycero-3-methoxypolyethylene glycol-2000 (DMG-PEG200). These components have previously been used in LNPs formula ted for the in vivo delivery of modified mRNAs in BNT162b2, a U.S. Food and Drug Adminstration (FDA)-approved SARS-CoV-2 vaccine (16).

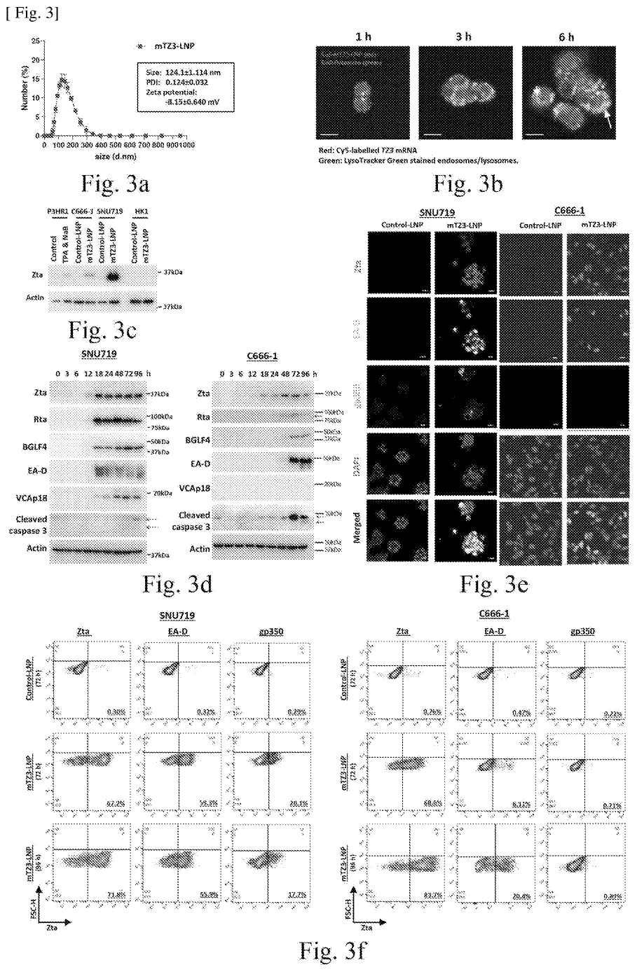

Using dynamic light scattering analysis (DLS), the size of the formulated LNP encapsulated TZ3 mRNA (mTZ3-LNP) s was determined to be approximately 124 nm, and its polydispersity index was 0.124±0.032 (FIG. 3a). The encapsulation efficiency of the synthesized mTZ3-LNP was as high as 90%. In FIG. 3b, we illustrate the uptake of mTZ3-LNPs by EBV positive SNU719 cells and the intracellular release of the TZ3 mRNAs using confocal laser scanning microscopy.

After 24 h of incubation with mTZ3-LNP, efficient induction of Zta expression was detected in EBV-positive SNU719 and C666-1 cells by western blotting (FIG. 3c). The induction of Zta expression was observed in SNU719 and C666-1 cells at 12 and 18 h post-mTZ3-LNP treatment respectively (FIG. 3d). Using quantitative reverse transcription-polymerase chain reaction (qRT-PCR), we revealed the upregulation of BZLF1 transcripts in SNU719 and C666-1 cells as early as 4 and 8 h post-mTZ3-LNP treatment, respectively. The induction of EBV immediately early (Rta), early (BGLF4, EA-D) and late (VCA) lytic proteins downstream of Zta was observed in these cells at later time points (FIG. 3d). The early lytic proteins BGLF4 and EA-D were expressed in C666-1 cells at 48-72 h post-mTZ3-LNP treatment. In SN719 cells, the expression of early (BGLF4, EA-D) and late (VCA) lytic proteins was induced at 18 h post-mTZ3-LNP treatment (FIG. 3d). The prevalence of SNU719 and C666-1 cells expressing Zta and downstream early lytic protein EA-D was demonstrated by immunofluorescence staining. Expression of the late lytic protein gp350 was found in the SNU719 cells at 96 h post-mTZ3-LNP treatment (FIG. 3e). Flow cytometry analysis detected up to 71.9% and 83.7% of Zta-positive tumor cells in mTZ3-LNP treated SNU719 and C666-1 cells respectively (FIG. 3f). Compared with Zta-positive cells, smaller proportions of EA-D- and gp350-positive cells were detected in the mTZ3-LNP treated SNU719 and C666-1 cells at 72 and 96 h post-treatment indicated the heterogenous status of lytic cycle in these lytic reactivated EBV-positive cancer cells. Notably, at 72-96 h post-treatment, caspase-3 cleavage was induced in mTZ3-LNP treated EBV-positive cancer cells, including C666-1 cells that experienced only abortive lytic cycle reactivation. These findings demonstrated that the synthesized mTZ3-LNPs produced functional TZ3 transcriptional activators that could induce BZLF1 expression in EBV-positive cancer cells.

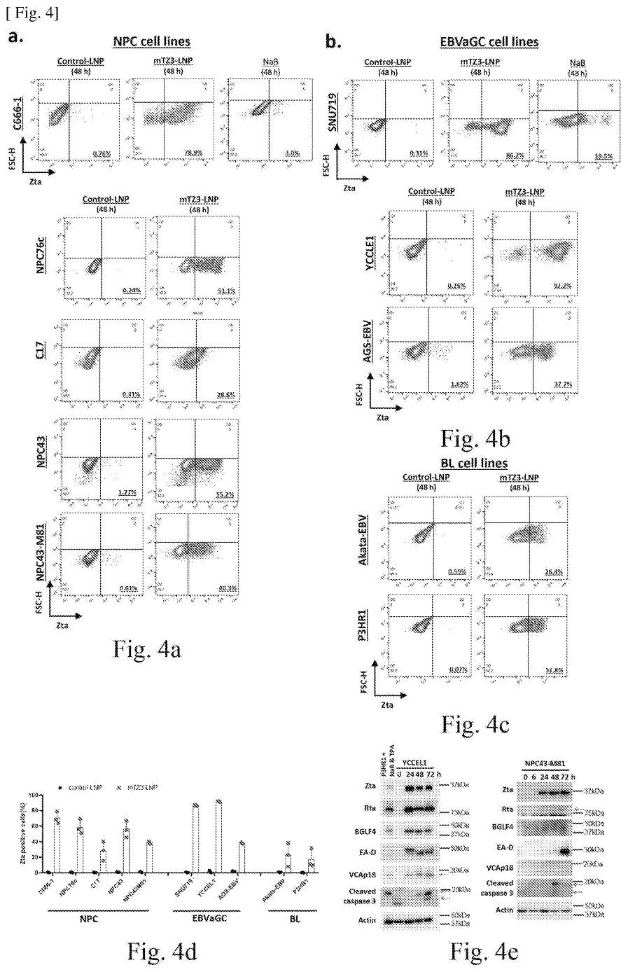

In addition to SNU719 and C666-1, the ability of mTZ3-LNP to reactivate Zta expression was tested in a panel of EBV-positive cancer models, including four NPC (C17, NPC43, NPC43-M81 and NPC76c), two EBVaGC (AGS-EBV and YCCLE1) and two BL (Akata-EBV and P3HR1) cell lines (FIG. 4). Strikingly, mTZ3-LNP efficiently induced Zta expression in all EBV-positive cancer cell lines. After 48 h of treatment with mTZ3-LNP, flow cytometry analysis revealed proportions of Zta-expressing cells ranging from approximately 26.4% to 92.2% in this panel of EBV-positive cancer models (FIG. 4a-d). The induced expression of Zta and its downstream EBV lytic proteins was also demonstrated in mTZ3-LNP-treated YCCLE1 and NPC43-M81 cells using western blotting (FIG. 4e). Compared with chemical activators, mTZ3-LNP reactivated the EBV lytic cycle in EBV-positive epithelial cancers much more efficiently. At 48 h post-mTZ-LNP treatment, we detected Zta expression in up to 86.2% and 78.9% of SNU719 and C666-1 cells, respectively, compared with only 19.5% and 3%, respectively, after sodium butyrate (NaB) treatment (FIG. 4a-b). Our study thus demonstrated that mTZ3-LNP is a potent lytic activator for different types of EBV-positive malignant cells.

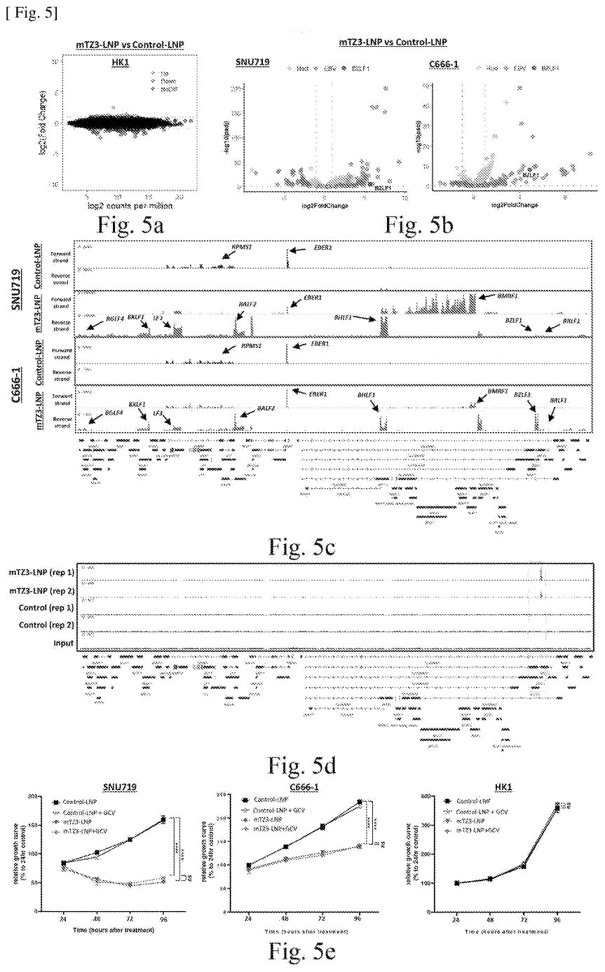

Using RNA-sequencing (RNA-seq), we revealed that few cellular genes were transcriptionally activated in EBV-negative HK1 cells treated with mT Z3-LNP, indicating the high specificity of the TZ3 transcriptional activator (FIG. 5a-b). We observed a similar result in HK1 cells transiently transfect ed with the TZ3 construct (FIG. 2e). Notably, the significant upregulation of EBV and other cellular transcripts was observed in mTZ3-LNP-treated SNU719 and C666-1 cells (FIG. 5b). In addition to BZLF1, EBV transcriptome profiling demonstrated the abundant expression of multiple lytic gene transcripts (e.g., BRLF1, BGLF4, BXLF1, LF3, BALF2, BHLF1, BMRF1) in EBV-positive tumor cells treated with mTZ3-LNP (FIG. 5c). Furthermore, mTZ3-LNP mediated BZLF1 expression and lytic reactivation induced the expression of multiple cellular genes in EBV-positive SNU719 and C666-1 cells. Despite the differentially expressed genes involved in multiple cellular mechanisms, few of them were detected in both mTZ3-LNP-treated SNU719 and C666-1 cells. These distinct transcription patterns may be attributable to unique genomic changes in each cell line and the abortive lytic cycle in C666-1 cells.

In addition to the RNA-seq study, we confirmed the absence of an off-target effect of mTZ3-LNP treatment via chromatin immunoprecipitation (ChIP)-sequencing analysis with an anti-FLAG antibody. As shown in FIG. 5d, the predicted TZ-binding sequences in the BZLF promoter from the EBV genome were enriched in mTZ3-LNP-treated EBV-positive SNU719 cells. ChIP-sequencing analysis did not identify any potential TZ3-binding sequences in the exon, intron and regulatory regions of human genes. Furthermore, the specific cytotoxicity of the synthetic mTZ3-LNP for EBV-positive cancer cells is also demonstrated in FIG. 5d. Specifically, a significant reduction in cell viability was observed in EBV-positive SNU719 and C666-1 cells treated with mTZ3-LNP. Notably, mTZ3-LNP neither alone nor in combination with GCV exerted a significant effect on the viability of the EBV-negative NPC cell line, HK1.

Reactivation of EBV Lytic Genes by In Vivo Delivery of mTZ3-LNP

The in vivo delivery of mRNA to EBV-positive tumors via the formulated ALC-0315-LNPs was examined in nonobese diabetic severe combined immunodeficiency disease (NOD-SCID) mouse models. For this purpose, luciferase mRNA-encapsulated LNPs were intravenously injected into mice. Notably, luciferase protein signals were detected in the tumors at 24 h after injection. No luciferase protein signals were found in other normal organs of the mice except for the liver. Through int ravenous injection of Dil C18-labeled LNP encapsulated TZ3 mRNA into NOD-SCID mice, we determined that the half-life of the formulated mTZ3-LNPs in circulation was 8.34 h. Dil C18 fluorescent signals were also detected in the tumor tissue from the mice at 3 h post-injection of Dil C18-labeled LNP encapsulated TZ3 mRNA.

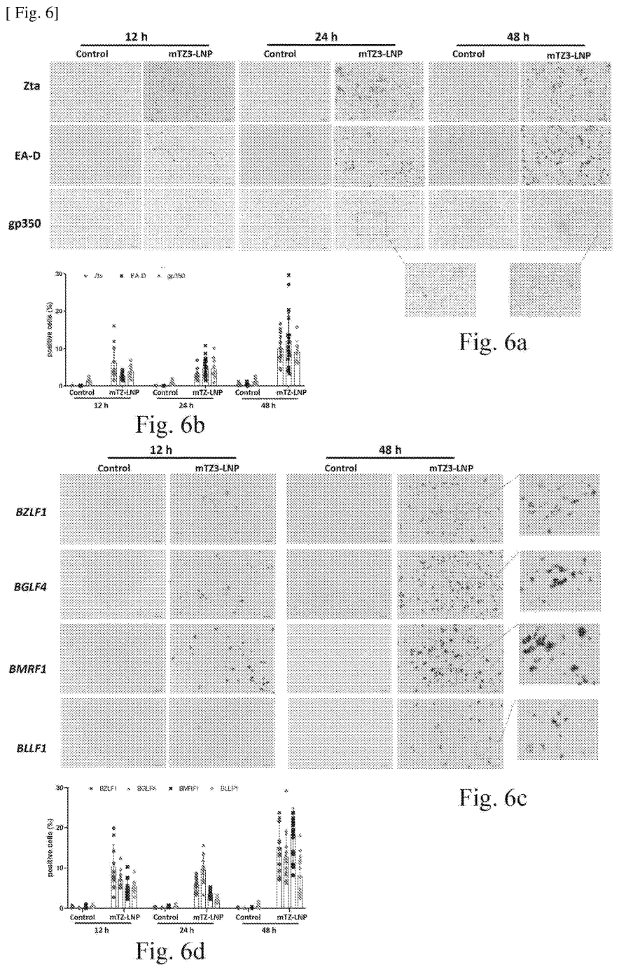

The efficient induction of endogenous Zta expression in EBVaGC and NPC tumors in vivo was demonstrated by the intravenous injection of mTZ3-LNP into NOD-SCID mice implanted with EBV-positive cancer cells. Using immunohistochemical staining, we revealed the obvious induction of Zta, EA-D/BMRF1 and gp350 expression in tumor cells from SNU719 xenografts at 12, 24 and 48 h post-injection of mTZ3-LNP; however, we did not observe similar findings in the controls (FIG. 6a). Expression of Zta, EA-D and gp350 lytic proteins was detected in 10%, 12% and 9% of tumor cells, respectively, at 48 h post-treatment. The findings indicated that, in vivo, mTZ3-LNP treatment successfully induced the expression of the early and late lytic proteins in EBV-positive tumor xenografts in NOD-SCID mouse models. In addition to BZLF1, the abundant transcription of other downstream lytic genes including BGLF4, BMRF1 and BLLF1 was detected in mTZ3-LNP treated tumors via RNAscope RNA in situ hybridization assays (FIG. 6c). Various amounts of immediate early or early lytic gene transcripts were observed in the tumor cells, indicating heterogeneity in terms of the lytic cycle stage. Our study demonstrated the transcriptional activation of BZLF1, BGLF4, BMRF1 and BLLF 1 genes in 15.1%, 12.7%, 18.3% and 8.1% of tumor cells, respectively, at 48 h post-intravenous injection of mTZ3-LNP (FIG. 6d). Notably, BGLF4 is not only a significant marker of lytic cycle progression but also encodes a serine/threonine kinase that converts the non-cytotoxic GCV to a cytotoxic form.

In Vivo Therapeutic Efficacy of mRNA-Based Lytic Induction Therapy

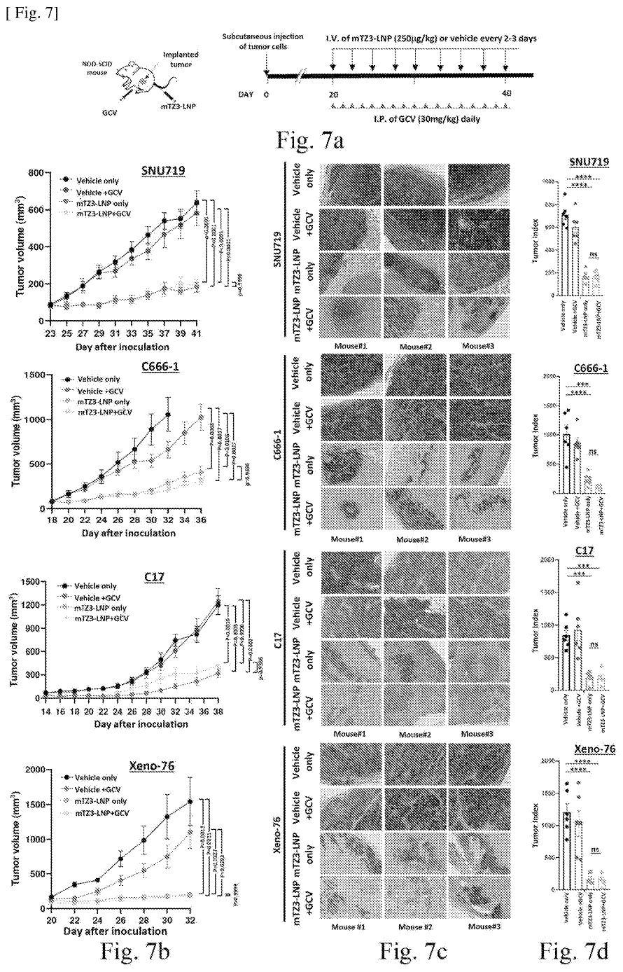

The therapeutic efficacy of combined mTZ3-LNP and GCV treatment for EBV-associated epithelial cancer was evaluated in NOD-SCID mouse models implanted with SNU719, C666-1 and C17 cells and an NPC patient-derived xenograft (PDX), Xeno-76. mRNA-based lytic induction treatment was started when the tumors sizes reach 80-100 mm³. mTZ3-LNP was injected through the tail vein every 2-3 days, whereas GCV was injected intraperitonially every day (FIG. 7a). As shown in FIG. 7b-d, potent growth inhibition of SNU719, C666-1, C17 and xeno-76 tumors was observed in mice treated with mTZ3-LNP alone or in combination with GCV compared with controls (P<0.005). Similar tumor growth inhibitory effects of mTZ3-LNP alone and in combination with GCV treatment were observed in all EBV-positive xenograft models. No obvious changes in the body weights of the mice were observed during treatment. Notably, the residual tumors harvested from mice with mTZ3-LNP treatment alone or in combination with GCV contained markedly fewer tumor cell components but increased proportions of necrotic lesions, lymphocytes and fibroblasts (FIG. 7b). The increased inflammation may be induced by EBV lytic re activation and ongoing cell death process although NOD-SCID mice lack of B and T lymphocytes. This observation reinforced our observation of the potent in vivo antitumor effects of mTZ3-LNP against EBV-positive cancers. In addition to the absence of changes in body weight, neither tissue damage to the organs in mice treated with mTZ3-LNP and GCV exhibited neither tissue damage to the organs nor significant changes in the serum concentrations of alanine aminotransferase (ALT), aspartate aminotransferase (AST) and creatinine, highlighting the safety of mTZ3-LNP-mediated lytic induction therapy in preclinical models of EBV-associated cancer.

### Discussion

The persistent latency in EBV-positive epithelial cancer cells is tightly controlled by acquired genetic changes and epigenetic modifications in the viral and host genomes. Multiple viral and cellular factors have been shown to regulate the latent-lytic switch to prevent cell death by lytic induction during transformation and clonal expansion (7-9). Over the past few decades, reports have described various drugs or reagents with lytic induction capacity in EBV-positive tumor cells. These chemical lytic inducers include histone deacetylase inhibitors (e.g., sodium butyrate, valproic acid, suberanilohydroxamic acid and romidepsin), DNA methyltransferase inhibitors (e.g., 5-aza-2′-deoxycytidine and 5-azacytidine), protein kinase C activators (e.g., TPA), chemotherapeutics agents (e.g., gemcitabine), antibacterial antibiotic (e.g., clofoctol) and several novel compounds (e.g., C7 and E11). These compounds reactivate EBV lytic genes through different mechanisms, targeting either epigenetic regulation or cellular signaling pathways (8). Although phase-I/II clinical trials of CLVA therapy with a combination of gemcitabine, valproic acid and valganciclovir treatment have been shown it to be safe and to elicit clinical responses in patients with recurrent NPC, response rates of less than 30% have been reported (10, 11). Previous in vitro studies have shown that the chemical lytic inducers elicit cell-context- and cell-type-specific lytic induction response from EBV-positive epithelia cancer cells. Most chemical lytic inducers reactivate EBV lytic genes much less efficiently in patient-derived EBV-positive epithelial cancer cell lines (e.g., SNU719, C666-1, NPC43, and C17) than in EBV re-infected cancer cells (e.g., HK1-EBV, AGS-BX1 and HONE-1-EBV) (17). The lytic induction response in EBV-infected tumor cells is believed to be influenced by epigenetic modifications and aberrant oncogenic signaling pathways acquired during clonal expansion. In addition to its low efficiency at lytic reactivation and intertumoral heterogeneity, the clinical implementation of chemical lytic inducers for EBV lytic induction therapy has been limited by the broad-spectrum cytotoxicity of these compounds. EBV lytic reactivation cannot be induced effectively at low doses of these drugs. At high doses, however, both EBV-infected and un-infected normal cells may be indiscriminately killed by the drugs.

To overcome the high level of complexity of EBV latent-lytic switch regulatory mechanisms, we developed a TALE-based transcriptional activator to artificially activate the expression of the EBV immediate early gene BZLF1 in EBV-positive tumor cells (FIG. 8). By exploiting a highly specific LNP-encapsulated mRNA encoding a TALE transcriptional activator, we demonstrated the potent in vivo antitumor effects of the artificial activation of endogenous BZLF1 expression in multiple models of EBV-positive epithelial cancer. We further demonstrated the highly specific cytotoxicity of this approach against to EBV-positive cells. The synthetic transcriptional activator was shown to specifically target the BZLF1 promoter in the EBV-positive cancer cells and did not induce transcriptional activity in EBV-negative cells. In addition to its reported growth arrest and cytotoxic effects, Zta can upregulate the transcription of various cellular genes contributing to multiple cancer hallmarks. Our approach avoids the safety issues caused by the cytotoxicity and potentially onco genic effects of ectopic Zta expression in non-infected cells (18).

Unlike the Casilio CRISPR-dCas9-based platform, which has three essential components, the in vivo delivery of a TALE transcription activator to primary and metastatic tumors of the patients using LNP-encapsulated mRNA technology is feasible. We exploited the LNP formulation used in an FDA-approved mRNA vaccine to develop a novel mRNA drug, mTZ3-LNP, for highly efficient lytic induction therapy against EBV-associated epithelial cancers. In our study, the safety and specificity of mTZ3-LNP were demonstrated in in vitro and in vivo preclinical models of EBVaGC and NPC. In addition to its capability for specific binding to the targeted-sequence, the strong transcriptional activity of the TALE transcriptional activator, TZ3, may be due to the high copy number of EBV episomes and the hypomethylated BZLF1 promoter in EBV-positive tumor cells (7-9). Importantly, once its expression is induced by the mTZ3-LNP, Zta can either cis- or trans-activate the BZLF1 promoters of multiple EBV episomes in tumor cells. Zta also drives the expression of BRLF1, which encodes the transactivator Rta; in turn, Rta induces BZLF1 transcription, forming a positive feedback loop to activate the BZLF1 promoter. Both mechanisms maintain the activation of BZLF1 and the accumulation of Zta after the transient expression of synthetic TZ3 transcriptional activators in EBV-positive tumor cells (9). In C17, an NPC cell line containing only 2-3 copies of the EBV genomes per cell, treatment with artificial activators also induced expression of Zta. As demonstrated by our studies, the delayed transcriptional activation of BZLF1 and downstream lytic genes was observed in C17 cells after Dox treatment. This finding indicates that tumor cells with a low copy number of EBV genome copy number require a longer period of Zta accumulation to activate the expression of downstream early and late lytic proteins. Notably, a significant growth inhibitory effect was observed in C17 CDX models in vivo following the administration of multiple mRNA-TZ3 doses. In addition, the high efficiency of mTZ3-LNP in inducing lytic reactivation in different types of EBV-associated cancers is attributable to its mechanism of action, which is not dependent on the host's epigenetic status or aberrant signaling pathways.

Using a panel of tumor xenograft models, our in vivo study highlighted the therapeutic efficacy of this newly developed mRNA-based lytic induction therapy against EBV-positive epithelial cancers. The safety of the intravenous administration of mTZ3-LNP for long-term treatment was demonstrated in NOD-SCID mouse xenograft models. For clinical implementation of lytic induction therapy, co-admiration with GCV is essential for the rapid and specific killing of EBV-positive tumor cells and inhibition of the infective virion production. Through in vivo study, we observed no significant difference in growth inhibition between tumors treated with mTZ3-LNP alone or in combination with GCV. The absence of an obvious bystander effect with combined treatment may be due to the high efficiency of lytic reactivation and the potent cytotoxic effect of mTZ3-LNP on EBV-positive tumors. Despite of the limited bystander killing effect observed, the GCV ad ministration is an essential procedure for rapid and specific killing of the EBV-positive cells and inhibit the production of infective virions during EBV lytic induction treatment. In addition to the direct cytotoxic effects, the potent innate and adaptive immune responses induced by abundant immunogenic lytic proteins may contribute to the effective eradication of EBV-positive cancers during lytic induction therapy (19, 20).

It is noted that the NOD-SCID mouse model used in this study lacks B- and T-lymphocytes, which are key immune cells triggering the host's immune responses to the highly expressed EBV lytic antigens. Nevertheless, we observed the NK cells accumulated in the adjacent necrosis regions and infiltrated into the residue tumors in the mTZ3-LNP treated mouse models. In a future study, we will establish EBV-positive tumor xenografts in a humanized mouse model, allowing us to accurately elucidate the innate and adaptive immune responses induced by mTZ3-LNP treatment and the potential therapeutic effects of combined treatment with mTZ3-LNP and immune checkpoint blockade (21). Combination of mTZ3-LNP with immunotherapeutic strategies such as immune checkpoint blockade and NK cell therapy may further enhance the treatment response of patients with EBV-associated cancers.

Along with the use of mRNA COVID-19 vaccines during the pandemic, the development of other mRNA therapies against a wide range of human diseases, including cancers, is on the rise. In this proof-of-concept study, we successfully developed a first-in-class mRNA drug for lytic induction therapy against EBV-associated epithelial cancers. By exploiting nucleoside-modified mRNA technologies, non-viral delivery strategies and the TALE artificial activator system, we produced mTZ3-LNP, a highly efficient inducer of the lytic reactivation and selective killing of EBV-positive tumor cells. This newly designed mRNA nano medicine provides a promising clinical opportunity for lytic induction therapy against EBV-associated epithelial cancers.

### Methods

### Cell Lines and Patient-Derived Xenografts

The EBVaGC cell line SNU719 was obtained from the Korean Cell Line Bank, Seoul, Republic of Korea. The EBVaGC cell lines YCCEL1 and AGS-EBV were provided by Professors Qian Tao and Jun Yu from the Chinese University of Hong Kong respectively (22, 23). The EBV-positive NPC cell lines C666-1, C17, NPC43, NPC43-M81 and NPC76c were established in our laboratory (24-26). The cell lines were used in various in vitro experiments. Xeno-76, an EBV-positive NPC PDX, was used in our in vivo study (26). The EBV-negative NPC cell line HK1 was included as a control (27). Two EBV-positive BL cell lines, P3HR1 and Akata-EBV, maintained in our laboratories were also used in the study. Except for C17, NPC43, NPC43M81 and N PC76c, all of the cells were maintained in Roswell Park Memorial Institute (RPMI)-1640 medium (Sigma, St. Louis, MO, USA), supplemented with 10% fetal bovine serum (Gibco, Waltham, MA, USA). To maintain the growth of the C17 and NPC76c cell lines, 0.5 μM of Y-27632 (Enzo Life Sciences Inc., Farmingdale, NY, USA), a ROCK inhibitor, was added to the RPMI-1640 medium. All cell cultures and all biological experiments were performed at 37° C. under 5% CO₂. All of the cell lines used in this study were authenticated using short tandem repeat (STR) profiling and EBER in situ hybridization. All cells were tested for mycoplasma contamination by PCR using the primer set 5′-YGCCTGVGTAGTAYRYW CGC-3′ (MYCO5) and 5′-GCGGTGTGTACAARMCCCGA-3′ (MYCO3). No commonly misidentified cell lines listed in the International Cell Line Authentication Committee (ICLAC) were used in this study.

### Construction of TALE Plasmids

BZLF1 promoter-targeted TALEs were designed and constructed in vitro using the Golden Gate assembly protocol (Golden Gate TAL Effector Kit 2.0, #1000000024; Addgene, Watertown, MA, USA) as described previously (28). Based on the identified target sequences on the BZLF1 promoter, the amino acid sequences of the binding domain of TZ3 and other TALEs (TZ1, TZ2 and TZ4) were designed as shown in Tables 9-10. The construct ed TALEs were then fused with the p65 activation domain to enable transcription. In brief, a DNA sequence designed to express FLAG-NLS-lacZ-p65HSF1 was first cloned into the pcDNA3.1 vector. Customizable polymorphic amino acid repeats from the TAL effectors targeting the EBV BZLF1 promoter sequence were used to replace the lacZ using multiple round of Golden Gate cloning. The TZ3 plasmid or the other TALE plasmids (TZ1, TZ2 and TZ4) was either transiently transfected into the cells for expression or linearized for in vitro transcription.

Preparation of Nucleoside Modified mRNA

The TZ3 plasmid was linearized by XmaI digestion and purified. The linearized TZ3 plasmid was then used as the template under the regulation of the T7 promoter. The nucleoside modified mRNA was synthesized as described [29]. In brief, in vitro transcription was conducted using a T7 high yield RNA synthesis kit (New England BioLabs, Ipswich, MA, USA), with 100% replacement of UTP with N1meΨTP and 1 μg of template. The reactions were incubated at 37° C. for 4 h, followed by treatment with RNase free DNase I. Next, mRNA capping was performed using the Vaccinia Capping System (New England BioLabs), followed by the addition of 3′ poly (A)-tails using E. coli poly (A) polymerase (New England BioLabs). mRNA purification was performed using the Monarch RNA Cleanup Kit (New England BioLabs).

Preparation and Characterization of LNP-Encapsulated mRNA

mTZ3-LNPs were prepared by mixing an ethanol phase containing lipids with purified mTZ3 mRNA in an aqueous phase in a microfluidic device. In brief, the ethanol phase was prepared by solubilizing a mixture of the ionizable lipid ALC-0315 (Cayman Chemical, Ann Arbor, MI, USA), 1,2-distearoyl-sn-glycero-3-phosphocholine (DSPC; Avanti, Alabaster, AL, USA), cholesterol (Sigma) and 1, 2-dimyristoyl-rac-glycero-3-methoxypolyethylene glycol-2000 (DMG-PEG 2000; Avanti) at a molecular ratio of 50:10:38.5:1.5 in ethanol. The aqueous phase was prepared by diluting mTZ3 mRNA or luciferase mRNA (TriLink, San Diego, CA, USA) eight-fold in 200 mM acetate buffer (pH 5.0). Syringe pumps were used to mix the aqueous and ethanol phases at a ratio of 3:1. The resulting LNPs were dialyzed against 1× phosphate-buffered saline (PBS) in a 20,000 MWCO cassette (Invitrogen, Carlsbad, CA, USA) at 4° C. for 2 h. The encapsulation efficiency of the mRNA-LNPs was calculated as reported previously (29). In brief, the samples were treated with PBS buffer alone (as unencapsulated mRNA) or with 2% Triton X-100 (as total mRNA). The Qubit RNA HS Assay Kit (Invitrogen) was used, and the RNA concentrations were measured using a Qubit 4 Fluorometer (Invitrogen). The encapsulation efficiency (EE) was calculated using the following formula: EE=[1-(unencapsulated mRNA/total mRNA)×100%] (30). The average size, polydispersity index and Zeta potential of the formulated mTZ3-LNP were determined using adynamic light scattering method and the Zetasizer Nano ZS90 system (Malvern Panalytical, Worcestershire, UK). The samples were diluted with PBS before measurement. To investigate cell uptake of the formulated mTZ3-LNP, the LNP-encapsulated Cy5-labeled mTZ3 mRNAs were incubated with the SNU719 cells for 1, 3 and 6 h. The fluorescent signals emitted by Cy5-mTZ3-LNP and LysoTracker Green (Invitrogen) stained endosomes were measured using an LSM 880 confocal laser scanning microscope with the AxioObserver system (Zeiss, Jena, Germany).

The fluorescence signals were measured in three channels: Cy5, excitation/emission wavelength, (ex/em) 633/697 nm; Dnd-26, ex/em, 488/524 nm; and hoechst, ex/em 405/460 nm.

### Quantitative Real-Time PCR

Total RNA was extracted from the cells using TRIzol reagent (Thermo Fisher Scientific, Waltham, MA, USA). The extracted RNA was then re verse transcribed into complementary DNA using the RT Regent Kit with h gDNA Eraser (TaKaRa, Kyoto, Japan) and quantitative real-time PCR was performed using SYBR Green master mix (Thermo Fisher Scientific). The mRNA expression levels of the EBV lytic genes were normalized to those of GAPDH. The primer sequences are listed in Table 11.

### Western Blot Assay

Protein extracts were prepared using RIPA lysis buffer supplemented with protease inhibitor (Roche, Basel, Switzerland). The protein concentrations were determined using a protein assay reagent (Bio-Rad, Hercules, CA, USA) against a bovine serum albumin (BSA) standard curve. Equal amounts of proteins in each extract were separated by sodium dodecyl-sulfate polyacrylamide gel electrophoresis (SDS-PAGE) and transferred onto 0.45 μm nitrocellulose membranes. The blocked membranes were incubated with the appropriate primary antibodies. The primary antibodies used in this study include anti-BZLF1/Zta (BZ1, Santa Cruz Biotechnology, Dallas, TX, USA; 1:1000), anti-EA-D (1108-1, Santa Cruz Biotechnology; 1:1000); anti-BGLF4 (1:1000), VCAp18 (#PA1-73003, Invitrogen; 1:500); anti-cleaved caspase 3 (Asp175, Cell Signaling; 1:1000) and anti-Actin (13E5, Cell Signaling; 1:4000) antibodies. After being washed and incubated with secondary antibody. Signals in the blots were detected using the ChemiDoc Image system (Bio-Rad).

### Fluorescence-Activated Cell Sorting (FACS) Analysis

The cells treated with chemical inducers, mTZ3-LNP or control-LNP were trypsinized, collected, and washed with cold PBS. The cells were then fixed in freshly prepared 4% paraformaldehyde and permeabilized with 0.1% TritonX-100 in PBS. The cell pellets were then stained with an Alexa-647-conjugated mouse anti-BZLF1/Zta antibody (BZ1, Santa Cruz Biotechnology; 1:100), Alexa-594-conjugated anti-EA-D antibody (1108-1, Santa Cruz Biotechnology; 1:100) or Alexa-488-conjugated anti-gp350 antibody (Santa Cruz Biotechnology; 1:100) and analyzed using BD LSRFortessa Cell Analyzer (Becton Dickinson, Franklin Lakes, NJ, USA). The data were analyzed using FlowJo software, version 10 (FlowJo, LLC, Ashland, OR, USA).

### Immunofluorescence Staining

For immunofluorescence staining, the cells were seeded onto coverslips in 6-well plates the day before treatment. The cells were then washed with PBS, fixed in 4% paraformaldehyde and permeabilized with 0.1% Triton X-100 in PBS for 30 min. The cells were then incubated with the Alexa-647 conjugated anti-BZLF1 antibody (BZ1, Santa Cruz Biotechnology; 1:100), Alexa-594 conjugated anti-EA-D (1108-1, Santa Cruz Biotechnology; 1:100) or Alexa-488 conjugated anti-gp350 (0221, Santa Cruz Biotechnology; 1:100) for 2 h at room temperature in the dark. Finally, the stained cells were counterstained with DAPI and mounted on slides with h Dako fluorescence mounting medium (Agilent, Santa Clara, CA, USA). The images were processed using an LSM 880 confocal laser-scanning microscope with Zen software (Zeiss, Oberkochen, Germany).

### Immunohistochemical Staining

The expression of the Zta, EA-D and gp350 proteins in the tumor sections of EBV-positive xenografts was detected using immunohistochemical staining. In brief, 4 μm sections were obtained from tumors grown in mice treated with PBS, GCV, mTZ3-LNP or a combination of mTZ3-LNP and GCV. The paraffin-embedded sections were dewaxed, rehydrated, and washed with water. After antigen retrieval, the samples were incubated with an anti-BZLF1/Zta (BZ1, Santa Cruz Biotechnology; 1:100), anti-EA-D (1108-1, Santa Cruz Biotechnology; 1:100) or anti-gp350 (0221, Santa Cruz Biotechnology; 1:100) primary antibodies. The sections were then incubated with a horseradish peroxidase-labeled secondary antibody, developed with 3, 3′-diaminobenzidine and counterstained with hematoxylin (Sigma). The percentages of cells expressing EBV lytic proteins in the tumor sections from mice treated with mTZ3-LNP and controls were evaluated. Representative images were acquired using a Nikon ECLIPSE Ni-E microscope equipped with a Ds-Ri2 microscope camera and NIS-Elements software (Nikon, Tokyo, Japan). At least four different pictures obtained in each triplicate (×200 magnification) and analyzed using ImageJ software to determine the percentage of tumor cells expressing EBV lytic proteins.

### EBER In Situ Hybridization

EBV-positive cancer cells were detected in tumor specimens using by EBER in situ hybridization assay. An EBER probe ISH kit (Leica, Newcastle, U. K.) was us ed to confirm the presence of EBV in formalin-fixed paraffin-embedded (FFPE) tumor sections, according to the manufacturer's instructions.

### RNAscope RNA In Situ Hybridization

The expression of BZLF1 and a panel of lytic gene transcripts (BMRF1, BGLF4, and BLLF1) were detected using RNAscope 2.0 RISH assays and a panel of EBV lytic gene-specific probes (Advanced Cell Diagnostics, USA), as described previously (26). The percentages of cells expressing EBV lytic gene transcripts in the tumor sections of mice treated with mTZ3-LNP and controls were evaluated as described in the immunohistochemical staining section.

### Detection of Infectious EBV Particles

Culture supernatants from EBV-positive tumor cells induced to undergo artificial EBV lytic reactivation were harvested and centrifuged at 800 rpm for 5 min and then filtered through a 0.45 μm cellulose acetate filter to remove the cell debris. The centrifuged and filtered supernatants, which contained the EBV particles, were further subjected to ultracentrifugation at 20,000 rpm for 4 h at 4° C. to pellet the EBV particles. The supernatant was discarded, and the pellet was resuspended in RPMI-1640 medium supplemented with 10% fetal bovine serum at a volume 1/30 of the original supernatant volume. These procedures increased the EBV concentration in the culture supernatant by 30x. EBV-negative Akata cells were then incubated with the concentrated EBV supernatant for 3 days. The infected Akata cells were then harvested and subjected to DNA and RNA extraction to detect the presence of the EBV genome and EBV gene expression, respectively (25).

### RNA Sequencing

To evaluate the RNA profiles of the cancer cells after TZ3 transfection or mTZ3-LNP treatment, total RNA was extracted from the cells using TRIzol reagent (Invitrogen). RNA sequencing libraries were prepared using the Swift RNA Library Kit (Swift Biosciences, Ann Arbor, MI, USA) with DNase I treatment and rRNA and globin depletion. Next-generation sequencing (150 bp, pai red-end) was performed using an Illumina HiSeq1500 sequencing system (Illumina, San Diego, CA, USA). The adapter sequences and low-quality sequences in the total sequencing reads were filtered before downstream analysis. In brief, the reads were mapped, aligned and annotated to the human reference genome (GRCh38) and EBV genome (chrEBV_Akata_inverted) using Hisat2 (2.1.0) with the “--rna-strandness RF” parameter and StringTie (1.3.6) (32). Downstream analyses were performed on the R software platform (v4.1.0). Differentially expressed genes between the control and TZ3-treated samples were identified using DEseq2 (1.32.0) with the criterion of a false discovery rate below 0.05 (33). The expression levels of protein-coding genes were further determined via gene set enrichment analysis (GSEA), using Hallmark and GO: BP gene sets obtained from the Molecular Signature Database and the clusterProfiler package (4.0.5) (34-35). Volcano plots were generated using the ggplot2 package (3.4.1). The raw data in the fastq files of the RNA-sequencing have been deposited in Sequence Read Archive (RA) on National Center for Biotechnology Information (NCBI)) under accession number PRJNA1007461.

### Chromatin Immunoprecipitation Sequencing

The genomic specificity of the TALE transcriptional activator TZ3 was evaluated in mTZ3-LNP-treated C666-1 cells using ChIP-sequencing, as de scribed previously (36). mTZ3-LNP-treated and control cells were fix ed in 1% formaldehyde and quenched by glycine. Chromatin was prepared using a truCHIP Chromatin Shearing Kit (Covaris, Woburn, USA) and broken into 100-500 bp fragments using Covaris S220 Focused-ultrasonicator (Covaris). The protein-DNA complexes were immunoprecipitated using 5 μg of an anti-FLAG antibody (F1804, Sigma) on a rotator at 4° C. overnight and then purified using magnetic beads (26162, Pierce; ThermoFisher). After washing, crosslink reversal and DNA purification were performed, and 8 ng of immunoprecipitate and input DNA were used for each Illumina sequencing library construction ac cording to the manufacturer's protocol (Kapa Hyper Prep Kit, KK8504, Roche). Each library was sequenced on a Nextseq 500 platform (Illumina) to obtain 150 base paired-end reads. The sequencing tags were mapped against the Akata reference genome (accession no. KC207813) using Bowtie 2. Unique FLAG-tag mapper tags were used for broad peak calling by MACS2 analysis. The raw data in the fastq files of the ChIP-sequencing have been deposited in Sequence Read Archive (RA) on National Center for Biotechnology Information (NCBI)) under accession number PRJNA1007461.

### EMSA

EMSA was performed to determine the binding of TZ3 transactivator to its target sequence in the BZLF1 promoter in the NPC cell line C666-1, as described previously (37). In addition to the wild-type sequence, three mutant sequences were included to demonstrate the binding specificity.

### Cell Viability Determination

Approximately 10⁴ cells per well per 100 μL were seeded in 96-well plates the day before transfection with the TZ3 plasmid or treatment with mTZ3-LNP and GCV. For mTZ3-LNP treatment, 100 ng of mRNA per well was added to the 96-well plate the next day, with or without 10 g/mL GCV in a total volume of 100 μL. At the end of the treatment, the medium was refreshed and 10 μl of CCK-8 reagent (Dojindo Molecular Technologies, Rockville, MD, USA) was added to each well to determine the cell viability. After incubation at 37° C. for 3-4 h, the absorbance was measured at 450 nm and 650 nm using a 96-well SpectraMax plate reader (Molecular Devices, San Jose, CA, USA). The cell growth inhibition in each well was calculated as follows: (viability_{control}−viability_{drug})/viability_{control}×100%. Each sample was analyzed in triplicate.

### Cell Cycle Analysis

The cells were detached from the culture plates using trypsin, washed with cold PBS, and fixed in 70% ethanol at 4° C. overnight. The cells were then washed with PBS and incubated with propidium iodide (1 μg/mL; Invitrogen, P3566) and RNase (10 μg/mL; Roche) for 30-min. After washing, 10⁴ cells per sample were analyzed using a FACSCalibur Flow Cytometer (BD Biosciences) to detect the DNA content. FlowJo software was used for data analysis.

### In Vivo Mouse Experiments

Minced Xeno-76 PDX tumor tissues or 5×10⁶ SNU719 or C666-1 cells were subcutaneously inoculated into the flanks of 3-4-week-old NOD-SCID mice with an initial body weight of approximately 18-22 g; the tumors were allowed to grow to ~100 mm³. The mice were housed in the following conditions: temperature of 20° C.-23° C., relative humidity of 40%-60% and a 12-h light/dark cycle (7:00 a. m.-7:00 p. m.). The mice were randomly assigned to different experimental groups and intravenously injected with either the vehicle (PBS) or mTZ3-LNP every 2 days for 2 weeks. GCV was intraperitoneally injected into the mice in the GCV only group and the combined mTZ3-LNP and GCV group. The mice were weighed, and their tumors were measured with a caliper every 3 days. When the tumor sizes exceeded 1000 mm³, the mice were killed, and tumor and blood samples were collected for analysis. The tumor volume was calculated using the formula 0.5×1×w², where 1 and w represent the tumor length and width, respectively. Serum samples and organs, including the heart, lung, liver, spleen, and kidney, were collected at the end of the experiment to evaluate the in vivo cytotoxicity of the mT Z3-LNP treatment. The serum aspartate transaminase (ALT), alkaline phosphatase (AST), and creatinine concentrations were measured. Formalin-fixed paraffin-embedded sections of the organs were subjected to hematoxylin and eosin (H&E) staining. The histological features were assessed by a pathologist (KF To).

To evaluate the circulation lifetime of the LNP-encapsulated mTZ3 mRNA in circulation in NOD-SCID mouse models, LNPs were fluorescently labelled with Dil C18 (Invitrogen). Four mice per group were intravenously injected with either Dil C18-labeled LNP-encapsulated mTZ3 mRNA at a dose of 0.5 mg RNA/kg or PBS as a control. Fifty microliters of blood were collected in the EDTA-treated tubes from the facial vein at 0, 8, 24 and 48 h post-injection. The circulating LNPs were measured by detecting the Dil C18 fluorescent signals in the blood samples, and the plasma was extracted. The circulating LNP were measured by detecting the Dil C18 signals in the plasma using a SpectraMax plate reader (Molecular Devices) (38). All animal care and experimental procedures were approved by the University Animal Experimentation Ethics Committee (AEEC), the Chinese University of Hong Kong.

### Statistical Analysis

All graphs were generated using GraphPad 8 software (GraphPad Inc, San Diego, CA, USA), and all statistical analyses were performed using one-way analysis of variance (ANOVA) or two-sided Student's t-tests. All in vitro experiments were performed in triplicate. Error bars indicate the standard deviation (S. D.) unless otherwise specified to represent the standard error of the mean (S EM). A P value<0.05 was considered to indicate statistical significance.

All patents, patent applications, and other publications, including GenBank Accession Numbers and equivalents, cited in this application are incorporated by reference in the entirety for all purposes.

### REFERENCES

1. Khan, G., Fitzmaurice, C., Naghavi, M., Ahmed, L. A. Global and regional incidence, mortality and disability-adjusted life-years for Epstein-Barr virus-attributable malignancies, 1990-2017. BMJ Open. 10, e037505 (2020).
2. Young, L. S., Yap, L. F., Murray, P. G. Epstein-Barr virus: more than 50 years old and still providing surprises. Nat Rev Cancer. 16, 789-802 (2016).
3. Damania, B., Kenney, S. C., Raab-Traub, N. Epstein-Barr virus: biology and clinical disease. Cell. 185, 3652-70 (2022).
4. Rodriguez, A., Armstrong, M., Dwyer, D., Flemington, E. Genetic dissection of cell growth arrest functions mediated by the Epstein-Barr virus lytic gene product, Zta. J Virol. 73, 9029-38 (1999).
5. Jung, E. J., Lee, Y. M., Lee, B. L., Chang, M. S., Kim, W. H. Lytic in duction and apoptosis of Epstein-Barr virus-associated gastric cancer cell line with epigenetic modifiers and ganciclovir. Cancer Lett. 247, 77-83 (2007).
6. Hui, K. F., et al. Activation of lytic cycle of Epstein-Barr virus by suberoylanilide hydroxamic acid leads to apoptosis and tumor growth sup pression of nasopharyngeal carcinoma. Int J Cancer. 131, 1930-40 (2012).
7. Hau, P. M., et al. Targeting Epstein-Barr virus in nasopharyngeal carcinoma. Front Oncol. 10, 600 (2020).
8. Yiu, S. P. T, Dorothea, M., Hui, K. F., Chiang, A. K. S. Lytic Induction therapy against Epstein-Barr virus-associated malignancies: past, pre sent, and future. Cancers (Basel). 12, 2142 (2020).
9. Mckenzie, J., El-Guindy, A. Epstein-Barr virus lytic cycle reactivation. Curr Top Microbiol Immunol. 391, 237-61 (2015).
10. Wildeman, M. A., et al. Cytolytic virus activation therapy for Epstein-Barr virus-driven tumors. Clin Cancer Res. 18, 5061-70 (2012).
11. Stoker, S. D., et al. Epstein-Barr virus-targeted therapy in nasopharyngeal carcinoma. J Cancer Res Clin Oncol. 141, 1845-57 (2015).
12. Cheng, A. W., et al. Casilio: a versatile CRISPR-Cas9-Pumilio hybrid for gene regulation and genomic labeling. Cell Res. 26, 254-7 (2016).
13. Moore, R., Chandrahas, A., Bleris, L. Transcription activator-like effectors: a toolkit for synthetic biology. ACS Synth Biol. 3, 708-16 (2014).
14. Al-Mozaini, M, et al. Epstein-Barr virus BART gene expression. J Gen Virol. 90, 307-16 (2009).
15. Jin, Y., Xie, Z., Lu, G., Yang, S., Shen, K. Characterization of variants in the promoter of BZLF1 gene of EBV in nonmalignant EBV-associated diseases in Chinese children. Virol J. 7, 92 (2010).
16. Walsh, E. E., et al. Safety and immunogenicity of two RNA-based Covid-19 vaccine candidates. N Engl J Med. 383, 2439-50 (2020).
17. Novalić, Z., van Rossen, T. M., Greijer, A. E., Middeldorp, J. M. Agents and app roaches for lytic induction therapy of Epstein-Barr virus associated malignancies Med Chem (Los Angeles). 6, 449-66 (2016).
18. Germini, D., et al. Oncogenic properties of the EBV ZEBRA protein. Cancers (Basel). 12, 1479 (2020).
19. Deng, Y., Münz, C. Roles of lytic viral replication and co-infections in the oncogenesis and immune control of the Epstein-Barr virus. Cancers (Basel). 13, 2275 (2021).
20. Münz, C. Natural killer cell responses to human oncogenic γ-herpesvirus infections. Semin Immunol. 60, 101652 (2022).
21. Liu, W. N. et al. Successful targeting of PD-1/PD-L1 with chimeric antigen receptor-natural killer cells and nivolumab in a humanized mouse cancer model. Sci Adv. 8: eadd1187 (2022)
22. Kim, D. N., et al. Characterization of naturally Epstein-Barr virus-infected gastric carcinoma cell line YCCEL1. J Gen Virol. 94, 497-506 (2013).
23. Liang, Q., et al. Integrative identification of Epstein-Barr virus-associated mutations and epigenetic alterations in gastric cancer. Gastroenterology. 147, 1350-62 (2014).
24. Cheung, S. T., et al. Nasopharyngeal carcinoma cell line (C666-1) consistently harbouring Epstein-Barr virus. Int J Cancer. 83, 121-6 (1999).
25. Yip, Y. L., et al. Establishment of a nasopharyngeal carcinoma cell line capable of undergoing lytic Epstein-Barr virus reactivation. Lab Invest. 98, 1093-104 (2018).
26. Lin, W., et al. Establishment and characterization of new tumor xenografts and cancer cell lines from EBV-positive nasopharyngeal carcinoma. Nat Commun. 9, 4663 (2018).
27. Huang, D. P., et al. Establishment of a cell line (NPC/HK1) from a differentiated squamous carcinoma of the nasopharynx. Int J Cancer. 26, 127-32 (1980).
28. Cermak, T., et al. Efficient design and assembly of custom TALEN and other TAL effector-based constructs for DNA targeting. Nucleic Acids R es. 39, e82 (2011).
29. Miao, L., et al. Delivery of mRNA vaccines with heterocyclic lipids increases anti-tumor efficacy by STING-mediated immune cell activation. Nat Biotechnol. 37, 1174-85 (2019).
30. Miao, L., et al. Synergistic lipid compositions for albumin receptor mediated delivery of mRNA to the liver. Nat Commun. 11, 2424 (2020)
31. Patel, S., et al. Naturally-occurring cholesterol analogues in lipid nanoparticles induce polymorphic shape and enhance intracellular delivery of m RNA. Nat Commun. 11, 983 (2020).
32. Pertea, M., Kim, D., Pertea, G. M., Leek, J. T., Salzberg, S. L. Transcript-level expression analysis of RNA-seq experiments with HISAT, String Tie and Ballgown. Nat Protoc. 11, 1650-67 (2016).
33. Love, M. I., Huber, W., Anders, S. Moderated estimation of fold change and dispersion for RNA-seq data with DESeq2. Genome Biol. 15, 550 (2014).
34. Liberzon, A., et al. The molecular signatures database hallmark gene set collection. Cell Syst. 1, 417-25 (2015).
35. Wu, T., et al. 2021. clusterProfiler 4.0: A universal enrichment tool for interpreting omics data. Innovation. 2, 100141 (2021).
36. Tsang, D. P. F., et al. Yin Yang 1-mediated epigenetic silencing of tumour-suppressive microRNAs activates nuclear factor-KB in hepatocellular carcinoma. J Pathol. 238:651-64 (2016).
37. Chung, G. T., et al. Constitutive activation of distinct NF-κB signals in EBV-associated nasopharyngeal carcinoma. J Pathol. 231, 311-22 (2013).
38. Chander, N., et al. Lipid nanoparticle mRNA systems containing high levels of sphingomyelin engender higher protein expression in hepatic and extra-hepatic tissues. Mol Ther Methods Clin Dev. 30:235-245 (2023).

TABLE 1
Genome sequence based on Human Herpesvirus 4 Type 1 (NC_007605):
BZLF1 promoter TALE targeting regions

| | TALE targeting regions | Position (5′-3′) |
|---|---|---|
| mTZ1 | AGCAAAGGTGGCCGG (SEQ ID NO: 1) | 90881-90895 |
| mTZ2 | CCTCTGTGATGTCATGG (SEQ ID NO: 2) | 90960-90976 |
| mTZ3 | ATGCATGAGCCACAGG (SEQ ID NO: 3) | 91025-91040 |
| mTZ4 | GTCTAAAATAAGCTGG (SEQ ID NO: 4) | 91076-91091 |
| mTZ5 | AGACACTTCTGAAAACTGCCTCCT (SEQ ID NO: 5) | 91056-91079 |
| mTZ6 | GCATGAGCCACAGGCATTGCTAAT (SEQ ID NO: 6) | 91015-91038 |
| mTZ7 | GCTAATGTACCTCATAGACACACCT (SEQ ID | 90996-91020 |
| | NO: 7) | |
| mTZ8 | GTACCTCATAGACACACCTAAATTT (SEQ ID | 90990-91014 |
| | NO: 8) | |
| mTZ9 | AGCACGTCCCAAACCATGACAT (SEQ ID NO: 9) | 90968-90989 |
| mTZ10 | CTTTTAGAAACTATGCATGAGOCACAGG (SEQ ID NO: 10) | 91025-91052 |

TABLE 2
Genome sequence based on Human Herpesvirus 4 Type 1 (NC_007605):
BRLF1 promoter TALE targeting regions

| | TALE targeting regions | Position (5′-3′) |
|---|---|---|
| mTR1 | GCAATATTTCCTCCAGAAAGAGT (SEQ ID NO: 11) | 94113-94135 |
| mTR2 | GCCCCAGCCAGATGTTCAGGAACCAAAAT (SEQ | 94041-94069 |
| | ID NO: 12) | |
| mTR3 | GCCCACTCATACTTAAGCGAT (SEQ ID NO: 13) | 94003-94023 |
| mTR4 | ATTGCGATTGTCCCGCCCAT (SEQ ID NO: 14) | 93936-93955 |
| mTR5 | CTTGTCCTGTGATAAAATCGCTCATAAGCT (SEQ | 94078-94107 |
| | ID NO: 15) | |
| mTR6 | AGTTAATGCCCCAGCCAGATGTT (SEQ ID NO: 16) | 94054-94076 |
| mTR7 | CTCTGCTGCCCACTCATACTTAAGCGAT (SEQ ID | 94003-94030 |
| | NO: 17) | |
| mTR8 | ATATTGCGATTGTCCCGCCCAT (SEQ ID NO: 18) | 93936-93957 |
| mTR9 | GCGATTGTCCCGCCCATGCCAAT (SEQ ID NO: 19) | 93930-93952 |
| mTR10 | TAGCAGCTCTAAGAAATA (SEQ ID NO: 20) | 93146-93163 |

TABLE 3
Genome sequence based on Human Herpesvirus 4 Type 1 (NC_007605):
BGLF4 promoter TALE targeting regions

| | TALE targeting regions | Position (5′-3′) |
|---|---|---|
| mTG1 | GGCAAAACAGCTCTGCCTGCTGCCT (SEQ ID | 111899-111923 |
| | NO: 21) | |
| mTG2 | CTGCCTGCTGCCTCAGGAACCCTTT (SEQ ID | 111887-111911 |
| | NO: 22) | |
| mTG3 | GCCTCAGGAACCCTTTTGCATCACT (SEQ ID | 111878-111902 |
| | NO: 23) | |
| mTG4 | GCATCACTCGTGTGTGCCTTT (SEQ ID NO: 24) | 111865-111885 |
| mTG5 | GCTACATAAACAAAACCTAAACGCT (SEQ ID | 111833-111857 |
| | NO: 25) | |
| mTG6 | ACTCTACCCCACGTATGATCT (SEQ ID NO: 26) | 111696-111716 |
| mTG7 | GGTCACCGCGACCAAGACTCAATTT (SEQ ID | 111430-111454 |
| | NO: 27) | |
| mTG8 | CTGTTTGAAACACAGTTTACATCTG (SEQ ID | 111534-111558 |
| | NO: 28) | |
| mTG9 | AAGAGCCTCGGTAGAAAAG (SEQ ID NO: 29) | 111604-111622 |
| mTG10 | CTGTTTGAAACACAGTTTAC (SEQ ID NO: 30) | 111539-111558 |

TABLE 4
Sequence of synthetic mRNA activating BZLF1

| | |
|---|---|
| mTZ1 (SEQ ID NO: 31) | AUGGACUACAAGGAUCACGACGGUGACUAUAAGGAUCAUGACAUCGACUAUAAGGACGAUGACGAUAAGAUCGAUGGCGGAGGCGGAUCU |
| | 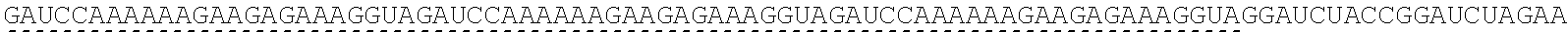 |
| | GAUCCAAAAAAGAAGAGAAAGGUAGAUCCAAAAAAGAAGAGAAAGGUAGAUCCAAAAAAGAAGAGAAAGGUAGGAUCUACCGGAUCUAGAA |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACAAUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACA |
| | UUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACAAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACAAUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACGGUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACAAUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAACAAU |
| | GGCGGCAAGCAAGCGCUCGAAACGGUGCAGCGGCUGUUGCCGGUGCUGUGCCAGGACCAUGGCCUGACUCCGGACCAAGUGGUGGCUAU |
| | CGCCAGCCACGAUGGCGGCAAGCAAGCGCUCGAAACGGUGCAGCGGCUGUUGCCGGUGCUGUGCCAGGACCAUGGCCUGACUCCGGACCA |
| | AGUGGUGGCUAUCGCCAGCCACGAUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAAUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAACAAUGGCGGCAAGCAAGCGCUCGAAAGCAUUGUGGCCCAGCUG |
| | AGCCGGCCUGAUCCGGCGUUGGCCGCGUUAACCAACGACCACCUCGUCGCCUUGGCCUGCCUCGGCGGACGUCCUGCCAUGGAUGCAGUGA |
| | AAAAGGGAUUGCCGCACGCGCCGGAAUUGAUCAGAAGAGUCAAUCGCCGUAUUGGCGAACGCACGUCCCAUCGCGUUGCCAGGGACCCAAA |
| | GAAGAAGCGCAAAGUGGAUCCUAAAAAGAAAAGAAAGGUAGGCGGCCGCGGGGGAGGCGGUUCCGGUGGCGGCGGAAGCGGAGGUGGAG |
| | GAUCAGGGCCGGCCGGAGGAGGUGGAAGCGGAGGAGGAGGAAGCGGAGGAGGAGGUAGCGGACCUAAGAAAAAGAGGAAGGUGGCGGCC |
| | GCUGGAUCCCCUUCAGGGCAGAUCAGCAACCAGGCCCUGGCUCUGGCCCCUAGCUCCGCUCCAGUGCUGGCCCAGACUAUGGUGCCCUCUA |
| | GUGCUAUGGUGCCUCUGGCCCAGCCACCUGCUCCAGCCCCUGUGCUGACCCCAGGACCACCCCAGUCACUGAGCGCUCCAGUGCCCAAGUCU |
| | ACACAGGCCGGCGAGGGGACUCUGAGUGAAGCUCUGCUGCACCUGCAGUUCGACGCUGAUGAGGACCUGGGAGCUCUGCUGGGGAACAGC |
| | ACCGAUCCCGGAGUGUUCACAGAUCUGGCCUCCGUGGACAACUCUGAGUUUCAGCAGCUGCUGAAUCAGGGCGUGUCCAUGUCUCAUAGU |
| | ACAGCCGAACCAAUGCUGAUGGAGUACCCCGAAGCCAUUACCCGGCUGGUGACCGGCAGCCAGCGGCCCCCCGACCCCGCUCCAACUCCCCUG |
| | GGAACCAGCGGCCUGCCUAAUGGGCUGUCCGGAGAUGAAGACUUCUCAAGCAUCGCUGAUAUGGACUUUAGUGCCCUGCUGUCACAGAUU |
| | UCCUCUAGUGGGCAGGGAGGAGGUGGAAGCGGCUUCAGCGUGGACACCAGUGCCCUGCUGGACCUGUUCAGCCCCUCGGUGACCGUGCCC |
| | GACAUGAGCCUGCCUGACCUUGACAGCAGCCUGGCCAGUAUCCAAGAGCUCCUGUCUCCCCAGGAGCCCCCCAGGCCUCCCGAGGCAGAGAA |
| | CAGCAGCCCGGAUUCAGGGAAGCAGCUGGUGCACUACACAGCGCAGCCGCUGUUCCUGCUGGACCCCGGCUCCGUGGACACCGGGAGCAAC |
| | GACCUGCCGGUGCUGUUUGAGCUGGGAGAGGGCUCCUACUUCUCCGAAGGGGACGGCUUCGCCGAGGACCCCACCAUCUCCCUGCUGACAG |
| | GCUCGGAGCCUCCCAAAGCCAAGGACCCCACUGUCUCCAUCGAUUGA |
| mTZ2 (SEQ ID NO: 32) | AUGGACUACAAGGAUCACGACGGUGACUAUAAGGAUCAUGACAUCGACUAUAAGGACGAUGACGAUAAGAUCGAUGGCGGAGGCGGAUCU |
| | 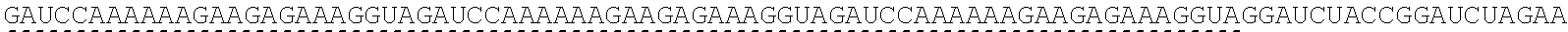 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACGGUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAAUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACAAUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACAUUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACGGUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAACAAU |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAACGGUGGCGGCAAGCAAGCGCUCGAAACGGUGCAGCGGCUGUUGCCGGUGCUGUGCCAGGACCAUGGCCUGACUCCGGACCA |
| | AGUGGUGGCUAUCGCCAGCCACGAUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAUUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAACGGUGGCGGCAAGCAAGCGCUCGAAACGGUGCAGCGGCUGUUG |
| | CCGGUGCUGUGCCAGGACCAUGGCCUGACCCCGGACCAAGUGGUGGCUAUCGCCAGCAACAAUGGCGGCAAGCAAGCGCUCGAAACGGUG |
| | CAGCGGCUGUUGCCGGUGCUGUGCCAGGACCAUGGCCUGACCCCGGACCAAGUGGUGGCUAUCGCCAGCAACAAUGGCGGCAAGCAAGCG |
| | CUCGAAAGCAUUGUGGCCCAGCUGAGCCGGCCUGAUCCGGCGUUGGCCGCGUUAACCAACGACCACCUCGUCGCCUUGGCCUGCCUCGGCG |
| | GACGUCCUGCCAUGGAUGCAGUGAAAAAGGGAUUGCCGCACGCGCCGGAAUUGAUCAGAAGAGUCAAUCGCCGUAUUGGCGAACGCACGU |
| | CCCAUCGCGUUGCCAGGGACCCAAAGAAGAAGCGCAAAGUGGAUCCUAAAAAGAAAAGAAAGGUAGGCGGCCGCGGGGGAGGCGGUUCCGG |
| | UGGCGGCGGAAGCGGAGGUGGAGGAUCAGGGCCGGCCGGAGGAGGUGGAAGCGGAGGAGGAGGAAGCGGAGGAGGAGGUAGCGGACCUA |
| | AGAAAAAGAGGAAGGUGGCGGCCGCUGGAUCCCCUUCAGGGCAGAUCAGCAACCAGGCCCUGGCUCUGGCCCCUAGCUCCGCUCCAGUGCU |
| | GGCCCAGACUAUGGUGCCCUCUAGUGCUAUGGUGCCUCUGGCCCAGCCACCUGCUCCAGCCCCUGUGCUGACCCCAGGACCACCCCAGUCAC |
| | UGAGCGCUCCAGUGCCCAAGUCUACACAGGCCGGCGAGGGGACUCUGAGUGAAGCUCUGCUGCACCUGCAGUUCGACGCUGAUGAGGACCU |
| | GGGAGCUCUGCUGGGGAACAGCACCGAUCCCGGAGUGUUCACAGAUCUGGCCUCCGUGGACAACUCUGAGUUUCAGCAGCUGCUGAAUCA |
| | GGGCGUGUCCAUGUCUCAUAGUACAGCCGAACCAAUGCUGAUGGAGUACCCCGAAGCCAUUACCCGGCUGGUGACCGGCAGCCAGCGGCCC |
| | CCCGACCCCGCUCCAACUCCCCUGGGAACCAGCGGCCUGCCUAAUGGGCUGUCCGGAGAUGAAGACUUCUCAAGCAUCGCUGAUAUGGACU |
| | UUAGUGCCCUGCUGUCACAGAUUUCCUCUAGUGGGCAGGGAGGAGGUGGAAGCGGCUUCAGCGUGGACACCAGUGCCCUGCUGGACCUGU |
| | UCAGCCCCUCGGUGACCGUGCCCGACAUGAGCCUGCCUGACCUUGACAGCAGCCUGGCCAGUAUCCAAGAGCUCCUGUCUCCCCAGGAGCCC |
| | CCCAGGCCUCCCGAGGCAGAGAACAGCAGCCCGGAUUCAGGGAAGCAGCUGGUGCACUACACAGCGCAGCCGCUGUUCCUGCUGGACCCCGG |
| | CUCCGUGGACACCGGGAGCAACGACCUGCCGGUGCUGUUUGAGCUGGGAGAGGGCUCCUACUUCUCCGAAGGGGACGGCUUCGCCGAGGA |
| | CCCCACCAUCUCCCUGCUGACAGGCUCGGAGCCUCCCAAAGCCAAGGACCCCACUGUCUCCAUCGAUUGA |
| mTZ3 (SEQ ID NO: 33) | AUGGACUACAAGGAUCACGACGGUGACUAUAAGGAUCAUGACAUCGACUAUAAGGACGAUGACGAUAAGAUCGAUGGCGGAGGCGGAUCU |
| | 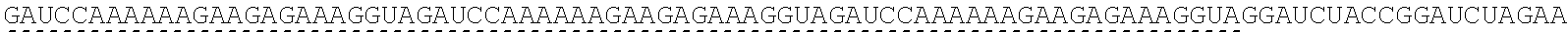 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAAUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACGGUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACAAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACAUUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACAAUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCCACGAU |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAACAUUGGCGGCAAGCAAGCGCUCGAAACGGUGCAGCGGCUGUUGCCGGUGCUGUGCCAGGACCAUGGCCUGACUCCGGACCA |
| | AGUGGUGGCUAUCGCCAGCCACGAUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAUUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAACAAUGGCGGCAAGCAAGCGCUCGAAACGGUGCAGCGGCUGUUG |
| | CCGGUGCUGUGCCAGGACCAUGGCCUGACCCCGGACCAAGUGGUGGCUAUCGCCAGCAACAAUGGCGGCAAGCAAGCGCUCGAAAGCAUU |
| | GUGGCCCAGCUGAGCCGGCCUGAUCCGGCGUUGGCCGCGUUAACCAACGACCACCUCGUCGCCUUGGCCUGCCUCGGCGGACGUCCUGCCA |
| | UGGAUGCAGUGAAAAAGGGAUUGCCGCACGCGCCGGAAUUGAUCAGAAGAGUCAAUCGCCGUAUUGGCGAACGCACGUCCCAUCGCGUUG |
| | CCAGGGACCCAAAGAAGAAGCGCAAAGUGGAUCCUAAAAAGAAAAGAAAGGUAGGCGGCCGCGGGGGAGGCGGUUCCGGUGGCGGCGGAA |
| | GCGGAGGUGGAGGAUCAGGGCCGGCCGGAGGAGGUGGAAGCGGAGGAGGAGGAAGCGGAGGAGGAGGUAGCGGACCUAAGAAAAAGAGG |
| | AAGGUGGCGGCCGCUGGAUCCCCUUCAGGGCAGAUCAGCAACCAGGCCCUGGCUCUGGCCCCUAGCUCCGCUCCAGUGCUGGCCCAGACUA |
| | UGGUGCCCUCUAGUGCUAUGGUGCCUCUGGCCCAGCCACCUGCUCCAGCCCCUGUGCUGACCCCAGGACCACCCCAGUCACUGAGCGCUCCA |
| | GUGCCCAAGUCUACACAGGCCGGCGAGGGGACUCUGAGUGAAGCUCUGCUGCACCUGCAGUUCGACGCUGAUGAGGACCUGGGAGCUCUG |
| | CUGGGGAACAGCACCGAUCCCGGAGUGUUCACAGAUCUGGCCUCCGUGGACAACUCUGAGUUUCAGCAGCUGCUGAAUCAGGGCGUGUCC |
| | AUGUCUCAUAGUACAGCCGAACCAAUGCUGAUGGAGUACCCCGAAGCCAUUACCCGGCUGGUGACCGGCAGCCAGCGGCCCCCCGACCCCGC |
| | UCCAACUCCCCUGGGAACCAGCGGCCUGCCUAAUGGGCUGUCCGGAGAUGAAGACUUCUCAAGCAUCGCUGAUAUGGACUUUAGUGCCCUG |
| | CUGUCACAGAUUUCCUCUAGUGGGCAGGGAGGAGGUGGAAGCGGCUUCAGCGUGGACACCAGUGCCCUGCUGGACCUGUUCAGCCCCUCG |
| | GUGACCGUGCCCGACAUGAGCCUGCCUGACCUUGACAGCAGCCUGGCCAGUAUCCAAGAGCUCCUGUCUCCCCAGGAGCCCCCCAGGCCUCC |
| | CGAGGCAGAGAACAGCAGCCCGGAUUCAGGGAAGCAGCUGGUGCACUACACAGCGCAGCCGCUGUUCCUGCUGGACCCCGGCUCCGUGGAC |
| | ACCGGGAGCAACGACCUGCCGGUGCUGUUUGAGCUGGGAGAGGGCUCCUACUUCUCCGAAGGGGACGGCUUCGCCGAGGACCCCACCAUCU |
| | CCCUGCUGACAGGCUCGGAGCCUCCCAAAGCCAAGGACCCCACUGUCUCCAUCGAUUGA |
| mTZ4 (SEQ ID NO: 34) | AUGGACUACAAGGAUCACGACGGUGACUAUAAGGAUCAUGACAUCGACUAUAAGGACGAUGACGAUAAGAUCGAUGGCGGAGGCGGAUCU |
| | 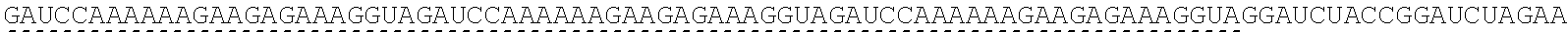 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACAUUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACAUUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACGGUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACAUUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAACAU |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACAAUGGCGGCAAGCAAGCGCUCGAAACGGUGCAGCGGCUGUUGCCGGUGCUGUGCCAGGACCAUGGCCUGACUCCGGACC |
| | AAGUGGUGGCUAUCGCCAGCCACGAUGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAACGGUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACAAUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAAUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |
| mTZ5 (SEQ ID NO: 35) | AUGGACUACAAGGAUCACGACGGUGACUAUAAGGAUCAUGACAUCGACUAUAAGGACGAUGACGAUAAGAUCGAUGGCGGAGGCGGAUCU |
| | 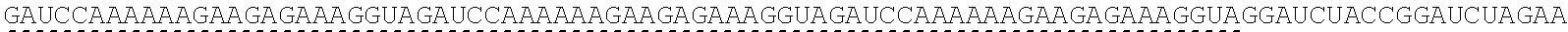 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAAUCACGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAUUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACUCCGGAC |
| | CAAGUGGUGGCUAUCGCCAGCCACGAUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACGGUGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACGGUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAAUCAC |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAACAUUGGCGGCAAGCAAGCGCUCGAAACGGUGCAGCGGCUGUUGCCGGUGCUGUGCCAGGACCAUGGCCUGACCCCGGACCA |
| | AGUGGUGGCUAUCGCCAGCAACAUUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAUUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAACAUUGGCGGCAAGCAAGCGCUCGAAACGGUGCAGCGGCUGUUG |
| | CCGGUGCUGUGCCAGGACCAUGGCCUGACUCCGGACCAAGUGGUGGCUAUCGCCAGCCACGAUGGCGGCAAGCAAGCGCUCGAAACGGUG |
| | CAGCGGCUGUUGCCGGUGCUGUGCCAGGACCAUGGCCUGACCCCGGACCAAGUGGUGGCUAUCGCCAGCAACGGUGGCGGCAAGCAAGCG |
| | CUCGAAACGGUGCAGCGGCUGUUGCCGGUGCUGUGCCAGGACCAUGGCCUGACCCCGGACCAAGUGGUGGCUAUCGCCAGCAAUCACGGC |
| | GGCAAGCAAGCGCUCGAAACGGUGCAGCGGCUGUUGCCGGUGCUGUGCCAGGACCAUGGCCUGACUCCGGACCAAGUGGUGGCUAUCGC |
| | CAGCCACGAUGGCGGCAAGCAAGCGCUCGAAACGGUGCAGCGGCUGUUGCCGGUGCUGUGCCAGGACCAUGGCCUGACUCCGGACCAAGU |
| | GGUGGCUAUCGCCAGCCACGAUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAACGGUGGCGGCAAGCAAGCGCUCGAAACGGUGCAGCGGCUGUUGCCGGUGCUGUGCCAG |
| | GACCAUGGCCUGACUCCGGACCAAGUGGUGGCUAUCGCCAGCCACGAUGGCGGCAAGCAAGCGCUCGAAACGGUGCAGCGGCUGUUGCCG |
| | GUGCUGUGCCAGGACCAUGGCCUGACUCCGGACCAAGUGGUGGCUAUCGCCAGCCACGAUGGCGGCAAGCAAGCGCUCGAAACGGUGCAG |
| | CGGCUGUUGCCGGUGCUGUGCCAGGACCAUGGCCUGACCCCGGACCAAGUGGUGGCUAUCGCCAGCAACGGUGGCGGCAAGCAAGCGCUC |
| | GAAACGGUGCAGCGGCUGUUGCCGGUGCUGUGCCAGGACCAUGGCGCUAUCGCCAGCAACAAUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |
| mTZ6 (SEQ ID NO: 36) | AUGGACUACAAGGAUCACGACGGUGACUAUAAGGAUCAUGACAUCGACUAUAAGGACGAUGACGAUAAGAUCGAUGGCGGAGGCGGAUCU |
| | 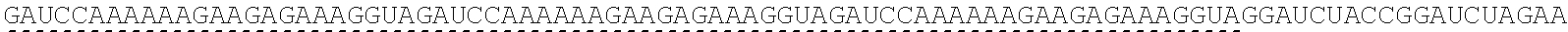 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAUUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAAUCACGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAAUCACGGCGGCAAGCAAGCGCUCGAAACGGUGCAGCGGCUGUUGCCGGUGCUGUG |
| | CCAGGACCAUGGCCUGACUCCGGACCAAGUGGUGGCUAUCGCCAGCCACGAUGGCGGCAAGCAAGCGCUCGAAACGGUGCAGCGGCUGUU |
| | GCCGGUGCUGUGCCAGGACCAUGGCCUGACUCCGGACCAAGUGGUGGCUAUCGCCAGCCACGAUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACAUUGGCGGCAAGCAAGC |
| | GCUCGAAACGGUGCAGCGGCUGUUGCCGGUGCUGUGCCAGGACCAUGGCCUGACUCCGGACCAAGUGGUGGCUAUCGCCAGCCACGAUG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAACAUUGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAAUCACGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAAUCACGGCGGCAAGCAAGCGCUCGAAACGGUGCAGCGGCUGUUGCCGGUGCUGUGCCA |
| | GGACCAUGGCCUGACUCCGGACCAAGUGGUGGCUAUCGCCAGCCACGAUGGCGGCAAGCAAGCGCUCGAAACGGUGCAGCGGCUGUUGCC |
| | GGUGCUGUGCCAGGACCAUGGCCUGACCCCGGACCAAGUGGUGGCUAUCGCCAGCAACAUUGGCGGCAAGCAAGCGCUCGAAACGGUGCA |
| | GCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACGGUGGC |
| | GGCAAGCAAGCGCUCGAAACGGUGCAGCGGCUGUUGCCGGUGCUGUGCCAGGACCAUGGCCUGACCCCGGACCAAGUGGUGGCUAUCGCC |
| | AGCAAUCACGGCGGCAAGCAAGCGCUCGAAACGGUGCAGCGGCUGUUGCCGGUGCUGUGCCAGGACCAUGGCCUGACUCCGGACCAAGUG |
| | GUGGCUAUCGCCAGCCACGAUGGCGGCAAGCAAGCGCUCGAAACGGUGCAGCGGCUGUUGCCGGUGCUGUGCCAGGACCAUGGCCUGACC |
| | CCGGACCAAGUGGUGGCUAUCGCCAGCAACGGUGGCGGCAAGCAAGCGCUCGAAACGGUGCAGCGGCUGUUGCCGGUGCUGUGCCAGGA |
| | CCAUGGCCUGACCCCGGACCAAGUGGUGGCUAUCGCCAGCAACAUUGGCGGCAAGCAAGCGCUCGAAACGGUGCAGCGGCUGUUGCCGGU |
| | GCUGUGCCAGGACCAUGGCCUGACCCCGGACCAAGUGGUGGCUAUCGCCAGCAACAUUGGCGGCAAGCAAGCGCUCGAAACGGUGCAGCG |
| | GCUGUUGCCGGUGCUGUGCCAGGACCAUGGCCUGACCCCGGACCAAGUGGUGGCUAUCGCCAGCAACGGUGGCGGCAAGCAAGCGCUCGA |
| | AACGGUGCAGCGGCUGUUGCCGGUGCUGUGCCAGGACCAUGGCGCUAUCGCCAGCAACAAUGGCGGCAAGCAAGCGCUCGAAAGCAUUG |
| | UGGCCCAGCUGAGCCGGCCUGAUCCGGCGUUGGCCGCGUUAACCAACGACCACCUCGUCGCCUUGGCCUGCCUCGGCGGACGUCCUGCCAU |
| | GGAUGCAGUGAAAAAGGGAUUGCCGCACGCGCCGGAAUUGAUCAGAAGAGUCAAUCGCCGUAUUGGCGAACGCACGUCCCAUCGCGUUGCC |
| | AGGGACCCAAAGAAGAAGCGCAAAGUGGAUCCUAAAAAGAAAAGAAAGGUAGGCGGCCGCGGGGGAGGCGGUUCCGGUGGCGGCGGAAGC |
| | GGAGGUGGAGGAUCAGGGCCGGCCGGAGGAGGUGGAAGCGGAGGAGGAGGAAGCGGAGGAGGAGGUAGCGGACCUAAGAAAAAGAGGAA |
| | GGUGGCGGCCGCUGGAUCCCCUUCAGGGCAGAUCAGCAACCAGGCCCUGGCUCUGGCCCCUAGCUCCGCUCCAGUGCUGGCCCAGACUAUG |
| | GUGCCCUCUAGUGCUAUGGUGCCUCUGGCCCAGCCACCUGCUCCAGCCCCUGUGCUGACCCCAGGACCACCCCAGUCACUGAGCGCUCCAGU |
| | GCCCAAGUCUACACAGGCCGGCGAGGGGACUCUGAGUGAAGCUCUGCUGCACCUGCAGUUCGACGCUGAUGAGGACCUGGGAGCUCUGCU |
| | GGGGAACAGCACCGAUCCCGGAGUGUUCACAGAUCUGGCCUCCGUGGACAACUCUGAGUUUCAGCAGCUGCUGAAUCAGGGCGUGUCCAU |
| | GUCUCAUAGUACAGCCGAACCAAUGCUGAUGGAGUACCCCGAAGCCAUUACCCGGCUGGUGACCGGCAGCCAGCGGCCCCCCGACCCCGCUC |
| | CAACUCCCCUGGGAACCAGGGGCCUGCCUAAUGGGCUGUCCGGAGAUGAAGACUUCUCAAGCAUCGCUGAUAUGGACUUUAGUGCCCUGCU |
| | GUCACAGAUUUCCUCUAGUGGGCAGGGAGGAGGUGGAAGCGGCUUCAGCGUGGACACCAGUGCCCUGCUGGACCUGUUCAGCCCCUCGGU |
| | GACCGUGCCCGACAUGAGCCUGCCUGACCUUGACAGCAGCCUGGCCAGUAUCCAAGAGCUCCUGUCUCCCCAGGAGCCCCCCAGGCCUCCCG |
| | AGGCAGAGAACAGCAGCCCGGAUUCAGGGAAGCAGCUGGUGCACUACACAGCGCAGCCGCUGUUCCUGCUGGACCCCGGCUCCGUGGACAC |
| | CGGGAGCAACGACCUGCCGGUGCUGUUUGAGCUGGGAGAGGGCUCCUACUUCUCCGAAGGGGACGGCUUCGCCGAGGACCCCACCAUCUCC |
| | CUGCUGACAGGCUCGGAGCCUCCCAAAGCCAAGGACCCCACUGUCUCCAUCGAUUGA |
| mTZ7 (SEQ ID NO: 37) | AUGGACUACAAGGAUCACGACGGUGACUAUAAGGAUCAUGACAUCGACUAUAAGGACGAUGACGAUAAGAUCGAUGGCGGAGGCGGAUCU |
| | 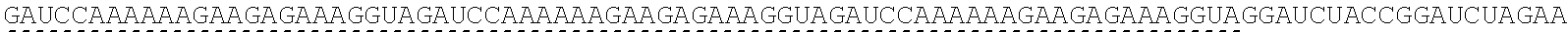 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACA |
| | UUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACGGUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAAUCACGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACGGUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAUUGGCGGCAAGCAAGCGCUCGAAACGGU |
| | GCAGCGGCUGUUGCCGGUGCUGUGCCAGGACCAUGGCCUGACUCCGGACCAAGUGGUGGCUAUCGCCAGCCACGAUGGCGGCAAGCAAGC |
| | GCUCGAAACGGUGCAGCGGCUGUUGCCGGUGCUGUGCCAGGACCAUGGCCUGACUCCGGACCAAGUGGUGGCUAUCGCCAGCCACGAUG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAACGGUGGCGGCAAGCAAGCGCUCGAAACGGUGCAGCGGCUGUUGCCGGUGCUGUGCCAGGACCAUGGCCUGACUCCGGACCAA |
| | GUGGUGGCUAUCGCCAGCCACGAUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACAUUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACGGUGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACAUUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAAUCACGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACAUUGGC |
| | GGCAAGCAAGCGCUCGAAACGGUGCAGCGGCUGUUGCCGGUGCUGUGCCAGGACCAUGGCCUGACUCCGGACCAAGUGGUGGCUAUCGC |
| | CAGCCACGAUGGCGGCAAGCAAGCGCUCGAAACGGUGCAGCGGCUGUUGCCGGUGCUGUGCCAGGACCAUGGCCUGACCCCGGACCAAGU |
| | GGUGGCUAUCGCCAGCAACAUUGGCGGCAAGCAAGCGCUCGAAACGGUGCAGCGGCUGUUGCCGGUGCUGUGCCAGGACCAUGGCCUGA |
| | CUCCGGACCAAGUGGUGGCUAUCGCCAGCCACGAUGGCGGCAAGCAAGCGCUCGAAACGGUGCAGCGGCUGUUGCCGGUGCUGUGCCAG |
| | GACCAUGGCCUGACCCCGGACCAAGUGGUGGCUAUCGCCAGCAACAUUGGCGGCAAGCAAGCGCUCGAAACGGUGCAGCGGCUGUUGCCG |
| | GUGCUGUGCCAGGACCAUGGCCUGACUCCGGACCAAGUGGUGGCUAUCGCCAGCCACGAUGGCGGCAAGCAAGCGCUCGAAACGGUGCAG |
| | CGGCUGUUGCCGGUGCUGUGCCAGGACCAUGGCCUGACUCCGGACCAAGUGGUGGCUAUCGCCAGCCACGAUGGCGGCAAGCAAGCGCUC |
| | GAAACGGUGCAGCGGCUGUUGCCGGUGCUGUGCCAGGACCAUGGCGCUAUCGCCAGCAACAAUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |
| mTZ8 (SEQ ID NO: 38) | AUGGACUACAAGGAUCACGACGGUGACUAUAAGGAUCAUGACAUCGACUAUAAGGACGAUGACGAUAAGAUCGAUGGCGGAGGCGGAUCU |
| | 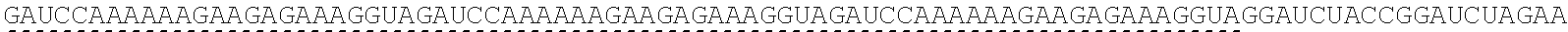 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAUUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACUCCGGACCAAGUGGUGGCU |
| | AUCGCCAGCCACGAUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACGGUGGCGGCAAGCAAGCGCUCGAAACGGUGCAGCGGCUGUUGCCGGUGCUGUGCCAGGACCAUGG |
| | CCUGACUCCGGACCAAGUGGUGGCUAUCGCCAGCCACGAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACAUUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACGGUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACAUUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAAUCAC |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAACAUUGGCGGCAAGCAAGCGCUCGAAACGGUGCAGCGGCUGUUGCCGGUGCUGUGCCAGGACCAUGGCCUGACUCCGGACCA |
| | AGUGGUGGCUAUCGCCAGCCACGAUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAUUGGCGGCAAGCAAGCGCUCGAAACGGUGCAGCGGCUGUUGCCGGUGCUGUGC |
| | CAGGACCAUGGCCUGACUCCGGACCAAGUGGUGGCUAUCGCCAGCCACGAUGGCGGCAAGCAAGCGCUCGAAACGGUGCAGCGGCUGUUG |
| | CCGGUGCUGUGCCAGGACCAUGGCCUGACCCCGGACCAAGUGGUGGCUAUCGCCAGCAACAUUGGCGGCAAGCAAGCGCUCGAAACGGUG |
| | CAGCGGCUGUUGCCGGUGCUGUGCCAGGACCAUGGCCUGACUCCGGACCAAGUGGUGGCUAUCGCCAGCCACGAUGGCGGCAAGCAAGCG |
| | CUCGAAACGGUGCAGCGGCUGUUGCCGGUGCUGUGCCAGGACCAUGGCCUGACUCCGGACCAAGUGGUGGCUAUCGCCAGCCACGAUGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAACGGUGGCGGCAAGCAAGCGCUCGAAACGGUGCAGCGGCUGUUGCCGGUGCUGUGCCAGGACCAUGGCCUGACCCCGGACCAAGU |
| | GGUGGCUAUCGCCAGCAACAUUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAACAUUGGCGGCAAGCAAGCGCUCGAAACGGUGCAGCGGCUGUUGCCGGUGCUGUGCCAG |
| | GACCAUGGCCUGACCCCGGACCAAGUGGUGGCUAUCGCCAGCAACAUUGGCGGCAAGCAAGCGCUCGAAACGGUGCAGCGGCUGUUGCCG |
| | GUGCUGUGCCAGGACCAUGGCCUGACCCCGGACCAAGUGGUGGCUAUCGCCAGCAACGGUGGCGGCAAGCAAGCGCUCGAAACGGUGCAG |
| | CGGCUGUUGCCGGUGCUGUGCCAGGACCAUGGCCUGACCCCGGACCAAGUGGUGGCUAUCGCCAGCAACGGUGGCGGCAAGCAAGCGCUC |
| | GAAACGGUGCAGCGGCUGUUGCCGGUGCUGUGCCAGGACCAUGGCGCUAUCGCCAGCAACAAUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |
| mTZ9 (SEQ ID NO: 39) | AUGGACUACAAGGAUCACGACGGUGACUAUAAGGAUCAUGACAUCGACUAUAAGGACGAUGACGAUAAGAUCGAUGGCGGAGGCGGAUCU |
| | 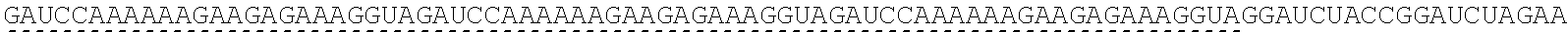 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAAUCACGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACA |
| | UUGGCGGCAAGCAAGCGCUCGAAACGGUGCAGCGGCUGUUGCCGGUGCUGUGCCAGGACCAUGGCCUGACUCCGGACCAAGUGGUGGCU |
| | AUCGCCAGCCACGAUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAAUCACGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACUCCGGACCAAGUGGUGGCUAUCGCCAGCCACGAUGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAACAU |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACAUUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAACAUUGGCGGCAAGCAAGCGCUCGAAACGGUGCAGCGGCUGUUGCCGGUGCUGUGCCAGGACCAUGGC |
| | CUGACUCCGGACCAAGUGGUGGCUAUCGCCAGCCACGAUGGCGGCAAGCAAGCGCUCGAAACGGUGCAGCGGCUGUUGCCGGUGCUGUG |
| | CCAGGACCAUGGCCUGACUCCGGACCAAGUGGUGGCUAUCGCCAGCCACGAUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAUUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACGGUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAAUCACGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAACAUUGGCGGCAAGCAAGCGCUCGAAACGGUGCAGCGGCUGUUGCCGGUGCUGUGCCAGGACCAUGGCCUGACUCCGGACCAAGU |
| | GGUGGCUAUCGCCAGCCACGAUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAACAUUGGCGGCAAGCAAGCGCUCGAAACGGUGCAGCGGCUGUUGCCGGUGCUGUGCCAG |
| | GACCAUGGCGCUAUCGCCAGCAACAAUGGCGGCAAGCAAGCGCUCGAAAGCAUUGUGGCCCAGCUGAGCCGGCCUGAUCCGGCGUUGGCC |
| | GCGUUAACCAACGACCACCUCGUCGCCUUGGCCUGCCUCGGCGGACGUCCUGCCAUGGAUGCAGUGAAAAAGGGAUUGCCGCACGCGCCGG |
| | AAUUGAUCAGAAGAGUCAAUCGCCGUAUUGGCGAACGCACGUCCCAUCGCGUUGCCAGGGACCCAAAGAAGAAGCGCAAAGUGGAUCCUAA |
| | AAAGAAAAGAAAGGUAGGGGGCCGCGGGGGAGGCGGUUCCGGUGGCGGCGGAAGCGGAGGUGGAGGAUCAGGGCCGGCCGGAGGAGGUG |
| | GAAGCGGAGGAGGAGGAAGCGGAGGAGGAGGUAGCGGACCUAAGAAAAAGAGGAAGGUGGCGGCCGCUGGAUCCCCUUCAGGGCAGAUCA |
| | GCAACCAGGCCCUGGCUCUGGCCCCUAGCUCCGCUCCAGUGCUGGCCCAGACUAUGGUGCCCUCUAGUGCUAUGGUGCCUCUGGCCCAGCC |
| | ACCUGCUCCAGCCCCUGUGCUGACCCCAGGACCACCCCAGUCACUGAGCGCUCCAGUGCCCAAGUCUACACAGGCCGGCGAGGGGACUCUGA |
| | GUGAAGCUCUGCUGCACCUGCAGUUCGACGCUGAUGAGGACCUGGGAGCUCUGCUGGGGAACAGCACCGAUCCCGGAGUGUUCACAGAUC |
| | UGGCCUCCGUGGACAACUCUGAGUUUCAGCAGCUGCUGAAUCAGGGCGUGUCCAUGUCUCAUAGUACAGCCGAACCAAUGCUGAUGGAGU |
| | ACCCCGAAGCCAUUACCCGGCUGGUGACCGGCAGCCAGCGGCCCCCCGACCCCGCUCCAACUCCCCUGGGAACCAGCGGCCUGCCUAAUGGGC |
| | UGUCCGGAGAUGAAGACUUCUCAAGCAUCGCUGAUAUGGACUUUAGUGCCCUGCUGUCACAGAUUUCCUCUAGUGGGCAGGGAGGAGGU |
| | GGAAGCGGCUUCAGCGUGGACACCAGUGCCCUGCUGGACCUGUUCAGCCCCUCGGUGACCGUGCCCGACAUGAGCCUGCCUGACCUUGACA |
| | GCAGCCUGGCCAGUAUCCAAGAGCUCCUGUCUCCCCAGGAGCCCCCCAGGCCUCCCGAGGCAGAGAACAGCAGCCCGGAUUCAGGGAAGCAG |
| | CUGGUGCACUACACAGCGCAGCCGCUGUUCCUGCUGGACCCCGGCUCCGUGGACACCGGGAGCAACGACCUGCCGGUGCUGUUUGAGCUGG |
| | GAGAGGGCUCCUACUUCUCCGAAGGGGACGGCUUCGCCGAGGACCCCACCAUCUCCCUGCUGACAGGCUCGGAGCCUCCCAAAGCCAAGGAC |
| | CCCACUGUCUCCAUCGAUUGA |
| mTZ10 (SEQ ID NO: 40) | AUGGACUACAAGGAUCACGACGGUGACUAUAAGGAUCAUGACAUCGACUAUAAGGACGAUGACGAUAAGAUCGAUGGCGGAGGGGGAUCU |
| | 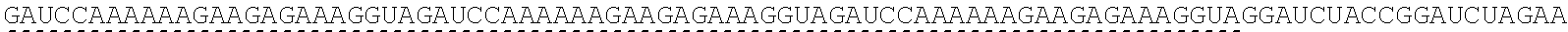 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACGGUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAAUCACGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAAUCACGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACGGUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACAUUGGCGGCAAGCAA |
| | GCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACUCCGGACCAAGUGGUGGCUA |
| | UCGCCAGCCACGAUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAACGGUGGCGGCAAGCAAGCGCUCGAAACGGUGCAGCGGCUGUUGCCGGUGCUGUGCCAGGACCAUGGC |
| | CUGACUCCGGACCAAGUGGUGGCUAUCGCCAGCCACGAUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACAUUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACGGUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACAUUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAAUCACGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAACAUUGGCGGCAAGCAAGCGCUCGAAACGGUGCAGCGGCUGUUGCCGGUGCUGUGCCAGGACCAUGGCCUGACUCCGGACCAAGU |
| | GGUGGCUAUCGCCAGCCACGAUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAACAUUGGCGGCAAGCAAGCGCUCGAAACGGUGCAGCGGCUGUUGCCGGUGCUGUGCCAG |
| | GACCAUGGCCUGACUCCGGACCAAGUGGUGGCUAUCGCCAGCCACGAUGGCGGCAAGCAAGCGCUCGAAACGGUGCAGCGGCUGUUGCCG |
| | GUGCUGUGCCAGGACCAUGGCCUGACCCCGGACCAAGUGGUGGCUAUCGCCAGCAACAUUGGCGGCAAGCAAGCGCUCGAAACGGUGCAG |
| | CGGCUGUUGCCGGUGCUGUGCCAGGACCAUGGCCUGACUCCGGACCAAGUGGUGGCUAUCGCCAGCCACGAUGGCGGCAAGCAAGCGCUC |
| | GAAACGGUGCAGCGGCUGUUGCCGGUGCUGUGCCAGGACCAUGGCCUGACUCCGGACCAAGUGGUGGCUAUCGCCAGCCACGAUGGCGG |
| | CAAGCAAGCGCUCGAAACGGUGCAGCGGCUGUUGCCGGUGCUGUGCCAGGACCAUGGCGCUAUCGCCAGCAACGGUGGCGGCAAGCAAGC |
| | GCUCGAAAGCAUUGUGGCCCAGCUGAGCCGGCCUGAUCCGGCGUUGGCCGCGUUAACCAACGACCACCUCGUCGCCUUGGCCUGCCUCGGC |
| | GGACGUCCUGCCAUGGAUGCAGUGAAAAAGGGAUUGCCGCACGCGCCGGAAUUGAUCAGAAGAGUCAAUCGCCGUAUUGGCGAACGCACG |
| | UCCCAUCGCGUUGCCAGGGACCCAAAGAAGAAGCGCAAAGUGGAUCCUAAAAAGAAAAGAAAGGUAGGCGGCCGCGGGGGAGGCGGUUCCG |
| | GUGGCGGCGGAAGCGGAGGUGGAGGAUCAGGGCCGGCCGGAGGAGGUGGAAGCGGAGGAGGAGGAAGCGGAGGAGGAGGUAGCGGACCU |
| | AAGAAAAAGAGGAAGGUGGCGGCCGCUGGAUCCCCUUCAGGGCAGAUCAGCAACCAGGCCCUGGCUCUGGCCCCUAGCUCCGCUCCAGUGC |
| | UGGCCCAGACUAUGGUGCCCUCUAGUGCUAUGGUGCCUCUGGCCCAGCCACCUGCUCCAGCCCCUGUGCUGACCCCAGGACCACCCCAGUCA |
| | CUGAGCGCUCCAGUGCCCAAGUCUACACAGGCCGGCGAGGGGACUCUGAGUGAAGCUCUGCUGCACCUGCAGUUCGACGCUGAUGAGGACC |
| | UGGGAGCUCUGCUGGGGAACAGCACCGAUCCCGGAGUGUUCACAGAUCUGGCCUCCGUGGACAACUCUGAGUUUCAGCAGCUGCUGAAUC |
| | AGGGCGUGUCCAUGUCUCAUAGUACAGCCGAACCAAUGCUGAUGGAGUACCCCGAAGCCAUUACCCGGCUGGUGACCGGCAGCCAGCGGCC |
| | CCCCGACCCCGCUCCAACUCCCCUGGGAACCAGCGGCCUGCCUAAUGGGCUGUCCGGAGAUGAAGACUUCUCAAGCAUCGCUGAUAUGGACU |
| | UUAGUGCCCUGCUGUCACAGAUUUCCUCUAGUGGGCAGGGAGGAGGUGGAAGCGGCUUCAGCGUGGACACCAGUGCCCUGCUGGACCUGU |
| | UCAGCCCCUCGGUGACCGUGCCCGACAUGAGCCUGCCUGACCUUGACAGCAGCCUGGCCAGUAUCCAAGAGCUCCUGUCUCCCCAGGAGCCC |
| | CCCAGGCCUCCCGAGGCAGAGAACAGCAGCCCGGAUUCAGGGAAGCAGCUGGUGCACUACACAGCGCAGCCGCUGUUCCUGCUGGACCCCGG |
| | CUCCGUGGACACCGGGAGCAACGACCUGCCGGUGCUGUUUGAGCUGGGAGAGGGCUCCUACUUCUCCGAAGGGGACGGCUUCGCCGAGGA |
| | CCCCACCAUCUCCCUGCUGACAGGCUCGGAGCCUCCCAAAGCCAAGGACCCCACUGUCUCCAUCGAUUGA |

TABLE 5
Sequence of synthetic mRNA activating BRLF1

| | |
|---|---|
| mTR1 (SEQ ID NO:41) | AUGGACUACAAGGAUCACGACGGUGACUAUAAGGAUCAUGACAUCGACUAUAAGGACGAUGACGAUAAGAUCGAUGGCGGAGGCGGAUCU |
| | 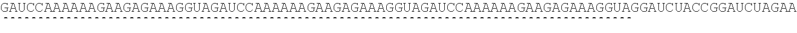 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAUUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACA |
| | UUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACGGUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACGGUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACGGUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACGGUGGCGGCAAGCAAGCGCUCGAAACGGUGCAGCGGCUGUUGCCGGUGCUGUGCCAGGACCAUGGCCUGACUCCGGACC |
| | AAGUGGUGGCUAUCGCCAGCCACGAUGGCGGCAAGCAAGCGCUCGAAACGGUGCAGCGGCUGUUGCCGGUGCUGUGCCAGGACCAUGGC |
| | CUGACUCCGGACCAAGUGGUGGCUAUCGCCAGCCACGAUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACAUUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAAUCACGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACAUUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAACAUUG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAACAUUGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAAUCACGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACAUUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAAUCACGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACGGUGGCGGCAAGCAAGCGCUCGAAAGCAUUG |
| | UGGCCCAGCUGAGCCGGCCUGAUCCGGCGUUGGCCGCGUUAACCAACGACCACCUCGUCGCCUUGGCCUGCCUCGGCGGACGUCCUGCCAU |
| | GGAUGCAGUGAAAAAGGGAUUGCCGCACGCGCCGGAAUUGAUCAGAAGAGUCAAUCGCCGUAUUGGCGAACGCACGUCCCAUCGCGUUGCC |
| | AGGGACCCAAAGAAGAAGCGCAAAGUGGAUCCUAAAAAGAAAAGAAAGGUAGGCGGCCGCGGGGGAGGCGGUUCCGGUGGCGGCGGAAGC |
| | GGAGGUGGAGGAUCAGGGCCGGCCGGAGGAGGUGGAAGCGGAGGAGGAGGAAGCGGAGGAGGAGGUAGCGGACCUAAGAAAAAGAGGAA |
| | GGUGGCGGCCGCUGGAUCCCCUUCAGGGCAGAUCAGCAACCAGGCCCUGGCUCUGGCCCCUAGCUCCGCUCCAGUGCUGGCCCAGACUAUG |
| | GUGCCCUCUAGUGCUAUGGUGCCUCUGGCCCAGCCACCUGCUCCAGCCCCUGUGCUGACCCCAGGACCACCCCAGUCACUGAGCGCUCCAGU |
| | GCCCAAGUCUACACAGGCCGGCGAGGGGACUCUGAGUGAAGCUCUGCUGCACCUGCAGUUCGACGCUGAUGAGGACCUGGGAGCUCUGCU |
| | GGGGAACAGCACCGAUCCCGGAGUGUUCACAGAUCUGGCCUCCGUGGACAACUCUGAGUUUCAGCAGCUGCUGAAUCAGGGCGUGUCCAU |
| | GUCUCAUAGUACAGCCGAACCAAUGCUGAUGGAGUACCCCGAAGCCAUUACCCGGCUGGUGACCGGCAGCCAGCGGCCCCCCGACCCCGCUC |
| | CAACUCCCCUGGGAACCAGCGGCCUGCCUAAUGGGCUGUCCGGAGAUGAAGACUUCUCAAGCAUCGCUGAUAUGGACUUUAGUGCCCUGCU |
| | GUCACAGAUUUCCUCUAGUGGGCAGGGAGGAGGUGGAAGCGGCUUCAGCGUGGACACCAGUGCCCUGCUGGACCUGUUCAGCCCCUCGGU |
| | GACCGUGCCCGACAUGAGCCUGCCUGACCUUGACAGCAGCCUGGCCAGUAUCCAAGAGCUCCUGUCUCCCCAGGAGCCCCCCAGGCCUCCCG |
| | AGGCAGAGAACAGCAGCCCGGAUUCAGGGAAGCAGCUGGUGCACUACACAGCGCAGCCGCUGUUCCUGCUGGACCCCGGCUCCGUGGACAC |
| | CGGGAGCAACGACCUGCCGGUGCUGUUUGAGCUGGGAGAGGGCUCCUACUUCUCCGAAGGGGACGGCUUCGCCGAGGACCCCACCAUCUCC |
| | CUGCUGACAGGCUCGGAGCCUCCCAAAGCCAAGGACCCCACUGUCUCCAUCGAUUGA |
| mTR2 (SEQ ID NO:42) | AUGGACUACAAGGAUCACGACGGUGACUAUAAGGAUCAUGACAUCGACUAUAAGGACGAUGACGAUAAGAUCGAUGGCGGAGGCGGAUCU |
| | 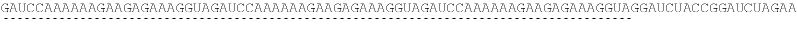 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACUCCGGACCAAGUGGUGGCU |
| | AUCGCCAGCCACGAUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAAUCACGGCGGCAAGCAAGCGCUCGAAACGGUGCAGCGGCUGUUGCCGGUGCUGUG |
| | CCAGGACCAUGGCCUGACUCCGGACCAAGUGGUGGCUAUCGCCAGCCACGAUGGCGGCAAGCAAGCGCUCGAAACGGUGCAGCGGCUGUU |
| | GCCGGUGCUGUGCCAGGACCAUGGCCUGACUCCGGACCAAGUGGUGGCUAUCGCCAGCCACGAUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACAUUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAAUCACGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAACAUUGGCGGCAAGCAAGCGCUCGAAACGGUGCAGCGGCUGUUGCCGGUGCUGUGCCAGGACCAUGGCCUGACCCCGGACCAAGU |
| | GGUGGCUAUCGCCAGCAACGGUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAAUCACGGCGGCAAGCAAGCGCUCGAAACGGUGCAGCGGCUGUUGCCGGUGCUGUGCCAGG |
| | ACCAUGGCCUGACCCCGGACCAAGUGGUGGCUAUCGCCAGCAACGGUGGCGGCAAGCAAGCGCUCGAAACGGUGCAGCGGCUGUUGCCGG |
| | UGCUGUGCCAGGACCAUGGCCUGACCCCGGACCAAGUGGUGGCUAUCGCCAGCAACGGUGGCGGCAAGCAAGCGCUCGAAACGGUGCAGC |
| | GGCUGUUGCCGGUGCUGUGCCAGGACCAUGGCCUGACUCCGGACCAAGUGGUGGCUAUCGCCAGCCACGAUGGCGGCAAGCAAGCGCUC |
| | GAAACGGUGCAGCGGCUGUUGCCGGUGCUGUGCCAGGACCAUGGCCUGACCCCGGACCAAGUGGUGGCUAUCGCCAGCAACAUUGGCGG |
| | CAAGCAAGCGCUCGAAACGGUGCAGCGGCUGUUGCCGGUGCUGUGCCAGGACCAUGGCCUGACCCCGGACCAAGUGGUGGCUAUCGCCAG |
| | CAAUCACGGCGGCAAGCAAGCGCUCGAAACGGUGCAGCGGCUGUUGCCGGUGCUGUGCCAGGACCAUGGCCUGACCCCGGACCAAGUGGU |
| | GGCUAUCGCCAGCAAUCACGGCGGCAAGCAAGCGCUCGAAACGGUGCAGCGGCUGUUGCCGGUGCUGUGCCAGGACCAUGGCCUGACCCC |
| | GGACCAAGUGGUGGCUAUCGCCAGCAACAUUGGCGGCAAGCAAGCGCUCGAAACGGUGCAGCGGCUGUUGCCGGUGCUGUGCCAGGACC |
| | AUGGCCUGACCCCGGACCAAGUGGUGGCUAUCGCCAGCAACAUUGGCGGCAAGCAAGCGCUCGAAACGGUGCAGCGGCUGUUGCCGGUG |
| | CUGUGCCAGGACCAUGGCCUGACUCCGGACCAAGUGGUGGCUAUCGCCAGCCACGAUGGCGGCAAGCAAGCGCUCGAAACGGUGCAGCGG |
| | CUGUUGCCGGUGCUGUGCCAGGACCAUGGCCUGACUCCGGACCAAGUGGUGGCUAUCGCCAGCCACGAUGGCGGCAAGCAAGCGCUCGAA |
| | ACGGUGCAGCGGCUGUUGCCGGUGCUGUGCCAGGACCAUGGCCUGACCCCGGACCAAGUGGUGGCUAUCGCCAGCAACAUUGGCGGCAA |
| | GCAAGCGCUCGAAACGGUGCAGCGGCUGUUGCCGGUGCUGUGCCAGGACCAUGGCCUGACCCCGGACCAAGUGGUGGCUAUCGCCAGCAA |
| | CAUUGGCGGCAAGCAAGCGCUCGAAACGGUGCAGCGGCUGUUGCCGGUGCUGUGCCAGGACCAUGGCCUGACCCCGGACCAAGUGGUGG |
| | CUAUCGCCAGCAACAUUGGCGGCAAGCAAGCGCUCGAAACGGUGCAGCGGCUGUUGCCGGUGCUGUGCCAGGACCAUGGCCUGACCCCGG |
| | ACCAAGUGGUGGCUAUCGCCAGCAACAUUGGCGGCAAGCAAGCGCUCGAAACGGUGCAGCGGCUGUUGCCGGUGCUGUGCCAGGACCAU |
| | GGCGCUAUCGCCAGCAACGGUGGCGGCAAGCAAGCGCUCGAAAGCAUUGUGGCCCAGCUGAGCCGGCCUGAUCCGGCGUUGGCCGCGUUA |
| | ACCAACGACCACCUCGUCGCCUUGGCCUGCCUCGGCGGACGUCCUGCCAUGGAUGCAGUGAAAAAGGGAUUGCCGCACGCGCCGGAAUUGA |
| | UCAGAAGAGUCAAUCGCCGUAUUGGCGAACGCACGUCCCAUCGCGUUGCCAGGGACCCAAAGAAGAAGCGCAAAGUGGAUCCUAAAAAGAA |
| | AAGAAAGGUAGGCGGCCGCGGGGGAGGCGGUUCCGGUGGCGGCGGAAGCGGAGGUGGAGGAUCAGGGCCGGCCGGAGGAGGUGGAAGCG |
| | GAGGAGGAGGAAGCGGAGGAGGAGGUAGCGGACCUAAGAAAAAGAGGAAGGUGGCGGCCGCUGGAUCCCCUUCAGGGCAGAUCAGCAACC |
| | AGGCCCUGGCUCUGGCCCCUAGCUCCGCUCCAGUGCUGGCCCAGACUAUGGUGCCCUCUAGUGCUAUGGUGCCUCUGGCCCAGCCACCUGC |
| | UCCAGCCCCUGUGCUGACCCCAGGACCACCCCAGUCACUGAGCGCUCCAGUGCCCAAGUCUACACAGGCCGGCGAGGGGACUCUGAGUGAAG |
| | CUCUGCUGCACCUGCAGUUCGACGCUGAUGAGGACCUGGGAGCUCUGCUGGGGAACAGCACCGAUCCCGGAGUGUUCACAGAUCUGGCCUC |
| | CGUGGACAACUCUGAGUUUCAGCAGCUGCUGAAUCAGGGCGUGUCCAUGUCUCAUAGUACAGCCGAACCAAUGCUGAUGGAGUACCCCGA |
| | AGCCAUUACCCGGCUGGUGACCGGCAGCCAGCGGCCCCCCGACCCCGCUCCAACUCCCCUGGGAACCAGCGGCCUGCCUAAUGGGCUGUCCG |
| | GAGAUGAAGACUUCUCAAGCAUCGCUGAUAUGGACUUUAGUGCCCUGCUGUCACAGAUUUCCUCUAGUGGGCAGGGAGGAGGUGGAAGC |
| | GGCUUCAGCGUGGACACCAGUGCCCUGCUGGACCUGUUCAGCCCCUCGGUGACCGUGCCCGACAUGAGCCUGCCUGACCUUGACAGCAGCC |
| | UGGCCAGUAUCCAAGAGCUCCUGUCUCCCCAGGAGCCCCCCAGGCCUCCCGAGGCAGAGAACAGCAGCCCGGAUUCAGGGAAGCAGCUGGUG |
| | CACUACACAGCGCAGCCGCUGUUCCUGCUGGACCCCGGCUCCGUGGACACCGGGAGCAACGACCUGCCGGUGCUGUUUGAGCUGGGAGAGG |
| | GCUCCUACUUCUCCGAAGGGGACGGCUUCGCCGAGGACCCCACCAUCUCCCUGCUGACAGGCUCGGAGCCUCCCAAAGCCAAGGACCCCACU |
| | GUCUCCAUCGAUUGA |
| mTR3 (SEQ ID NO: 43) | AUGGACUACAAGGAUCACGACGGUGACUAUAAGGAUCAUGACAUCGACUAUAAGGACGAUGACGAUAAGAUCGAUGGCGGAGGCGGAUCU |
| | 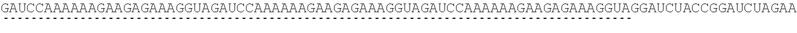 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAACAUUGGCGGCAAGCAAGCGCUCGAAACGGUGCAGCGGCUGUUGCCGGUGCUGUGCCAGGACCAUGGCCUGACUCCGGACCAA |
| | GUGGUGGCUAUCGCCAGCCACGAUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACGGUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACGGCGGCAAGCAAGCGCUCGAAACGGUGCAGCGGCUGUUGCCGG |
| | UGCUGUGCCAGGACCAUGGCCUGACCCCGGACCAAGUGGUGGCUAUCGCCAGCAACAUUGGCGGCAAGCAAGCGCUCGAAACGGUGCAGC |
| | GGCUGUUGCCGGUGCUGUGCCAGGACCAUGGCCUGACCCCGGACCAAGUGGUGGCUAUCGCCAGCAACGGUGGCGGCAAGCAAGCGCUC |
| | GAAACGGUGCAGCGGCUGUUGCCGGUGCUGUGCCAGGACCAUGGCCUGACCCCGGACCAAGUGGUGGCUAUCGCCAGCAACAUUGGCGG |
| | CAAGCAAGCGCUCGAAACGGUGCAGCGGCUGUUGCCGGUGCUGUGCCAGGACCAUGGCCUGACUCCGGACCAAGUGGUGGCUAUCGCCA |
| | GCCACGAUGGCGGCAAGCAAGCGCUCGAAACGGUGCAGCGGCUGUUGCCGGUGCUGUGCCAGGACCAUGGCCUGACCCCGGACCAAGUGG |
| | UGGCUAUCGCCAGCAACGGUGGCGGCAAGCAAGCGCUCGAAACGGUGCAGCGGCUGUUGCCGGUGCUGUGCCAGGACCAUGGCCUGACCC |
| | CGGACCAAGUGGUGGCUAUCGCCAGCAACGGUGGCGGCAAGCAAGCGCUCGAAACGGUGCAGCGGCUGUUGCCGGUGCUGUGCCAGGAC |
| | CAUGGCCUGACCCCGGACCAAGUGGUGGCUAUCGCCAGCAACAUUGGCGGCAAGCAAGCGCUCGAAACGGUGCAGCGGCUGUUGCCGGU |
| | GCUGUGCCAGGACCAUGGCCUGACCCCGGACCAAGUGGUGGCUAUCGCCAGCAACAUUGGCGGCAAGCAAGCGCUCGAAACGGUGCAGCG |
| | GCUGUUGCCGGUGCUGUGCCAGGACCAUGGCCUGACCCCGGACCAAGUGGUGGCUAUCGCCAGCAAUCACGGCGGCAAGCAAGCGCUCGA |
| | AACGGUGCAGCGGCUGUUGCCGGUGCUGUGCCAGGACCAUGGCCUGACUCCGGACCAAGUGGUGGCUAUCGCCAGCCACGAUGGCGGCA |
| | AGCAAGCGCUCGAAACGGUGCAGCGGCUGUUGCCGGUGCUGUGCCAGGACCAUGGCCUGACCCCGGACCAAGUGGUGGCUAUCGCCAGCA |
| | AUCACGGCGGCAAGCAAGCGCUCGAAACGGUGCAGCGGCUGUUGCCGGUGCUGUGCCAGGACCAUGGCCUGACCCCGGACCAAGUGGUG |
| | GCUAUCGCCAGCAACAUUGGCGGCAAGCAAGCGCUCGAAACGGUGCAGCGGCUGUUGCCGGUGCUGUGCCAGGACCAUGGCGCUAUCGCC |
| | AGCAACGGUGGCGGCAAGCAAGCGCUCGAAAGCAUUGUGGCCGCGUUAACCAACGACCACCUCGUCGCCUUGGCCUGCCUCGGCGGACGUC |
| | CUGCCAUGGAUGCAGUGAAAAAGGGAUUGCCGCACGCGCCGGAAUUGAUCAGAAGAGUCAAUCGCCGUAUUGGCGAACGCACGUCCCAUCG |
| | CGUUGCCAGGGACCCAAAGAAGAAGCGCAAAGUGGAUCCUAAAAAGAAAAGAAAGGUAGGCGGCCGCGGGGGAGGCGGUUCCGGUGGCGG |
| | CGGAAGCGGAGGUGGAGGAUCAGGGCCGGCCGGAGGAGGUGGAAGCGGAGGAGGAGGAAGCGGAGGAGGAGGUAGCGGACCUAAGAAAA |
| | AGAGGAAGGUGGCGGCCGCUGGAUCCCCUUCAGGGCAGAUCAGCAACCAGGCCCUGGCUCUGGCCCCUAGCUCCGCUCCAGUGCUGGCCCA |
| | GACUAUGGUGCCCUCUAGUGCUAUGGUGCCUCUGGCCCAGCCACCUGCUCCAGCCCCUGUGCUGACCCCAGGACCACCCCAGUCACUGAGCG |
| | CUCCAGUGCCCAAGUCUACACAGGCCGGCGAGGGGACUCUGAGUGAAGCUCUGCUGCACCUGCAGUUCGACGCUGAUGAGGACCUGGGAGC |
| | UCUGCUGGGGAACAGCACCGAUCCCGGAGUGUUCACAGAUCUGGCCUCCGUGGACAACUCUGAGUUUCAGCAGCUGCUGAAUCAGGGCGU |
| | GUCCAUGUCUCAUAGUACAGCCGAACCAAUGCUGAUGGAGUACCCCGAAGCCAUUACCCGGCUGGUGACCGGCAGCCAGCGGCCCCCCGACC |
| | CCGCUCCAACUCCCCUGGGAACCAGCGGCCUGCCUAAUGGGCUGUCCGGAGAUGAAGACUUCUCAAGCAUCGCUGAUAUGGACUUUAGUGC |
| | CCUGCUGUCACAGAUUUCCUCUAGUGGGCAGGGAGGAGGUGGAAGCGGCUUCAGCGUGGACACCAGUGCCCUGCUGGACCUGUUCAGCCC |
| | CUCGGUGACCGUGCCCGACAUGAGCCUGCCUGACCUUGACAGCAGCCUGGCCAGUAUCCAAGAGCUCCUGUCUCCCCAGGAGCCCCCCAGGC |
| | CUCCCGAGGCAGAGAACAGCAGCCCGGAUUCAGGGAAGCAGCUGGUGCACUACACAGCGCAGCCGCUGUUCCUGCUGGACCCCGGCUCCGU |
| | GGACACCGGGAGCAACGACCUGCCGGUGCUGUUUGAGCUGGGAGAGGGCUCCUACUUCUCCGAAGGGGACGGCUUCGCCGAGGACCCCACC |
| | AUCUCCCUGCUGACAGGCUCGGAGCCUCCCAAAGCCAAGGACCCCACUGUCUCCAUCGAUUGA |
| mTR4 (SEQ ID NO:44) | AUGGACUACAAGGAUCACGACGGUGACUAUAAGGAUCAUGACAUCGACUAUAAGGACGAUGACGAUAAGAUCGAUGGCGGAGGCGGAUCU |
| | 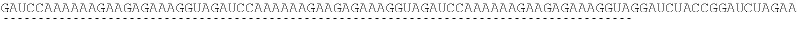 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAAUC |
| | ACGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAAUCACGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACAUUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACGGUGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACGGUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAAUCACGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACGGUGGC |
| | GGCAAGCAAGCGCUCGAAACGGUGCAGCGGCUGUUGCCGGUGCUGUGCCAGGACCAUGGCCUGACUCCGGACCAAGUGGUGGCUAUCGC |
| | CAGCCACGAUGGCGGCAAGCAAGCGCUCGAAACGGUGCAGCGGCUGUUGCCGGUGCUGUGCCAGGACCAUGGCCUGACUCCGGACCAAGU |
| | GGUGGCUAUCGCCAGCCACGAUGGCGGCAAGCAAGCGCUCGAAACGGUGCAGCGGCUGUUGCCGGUGCUGUGCCAGGACCAUGGCCUGA |
| | CUCCGGACCAAGUGGUGGCUAUCGCCAGCCACGAUGGCGGCAAGCAAGCGCUCGAAACGGUGCAGCGGCUGUUGCCGGUGCUGUGCCAG |
| | GACCAUGGCCUGACCCCGGACCAAGUGGUGGCUAUCGCCAGCAAUCACGGCGGCAAGCAAGCGCUCGAAACGGUGCAGCGGCUGUUGCCG |
| | GUGCUGUGCCAGGACCAUGGCCUGACUCCGGACCAAGUGGUGGCUAUCGCCAGCCACGAUGGCGGCAAGCAAGCGCUCGAAACGGUGCAG |
| | CGGCUGUUGCCGGUGCUGUGCCAGGACCAUGGCCUGACUCCGGACCAAGUGGUGGCUAUCGCCAGCCACGAUGGCGGCAAGCAAGCGCUC |
| | GAAACGGUGCAGCGGCUGUUGCCGGUGCUGUGCCAGGACCAUGGCCUGACUCCGGACCAAGUGGUGGCUAUCGCCAGCCACGAUGGCGG |
| | CAAGCAAGCGCUCGAAACGGUGCAGCGGCUGUUGCCGGUGCUGUGCCAGGACCAUGGCCUGACCCCGGACCAAGUGGUGGCUAUCGCCAG |
| | CAACAUUGGCGGCAAGCAAGCGCUCGAAACGGUGCAGCGGCUGUUGCCGGUGCUGUGCCAGGACCAUGGCGCUAUCGCCAGCAACGGUG |
| | GCGGCAAGCAAGCGCUCGAAAGCAUUGUGGCCCAGCUGAGCCGGCCUGAUCCGGCGUUGGCCGCGUUAACCAACGACCACCUCGUCGCCUU |
| | GGCCUGCCUCGGCGGACGUCCUGCCAUGGAUGCAGUGAAAAAGGGAUUGCCGCACGCGCCGGAAUUGAUCAGAAGAGUCAAUCGCCGUAU |
| | UGGCGAACGCACGUCCCAUCGCGUUGCCAGGGACCCAAAGAAGAAGCGCAAAGUGGAUCCUAAAAAGAAAAGAAAGGUAGGCGGCCGCGGG |
| | GGAGGCGGUUCCGGUGGCGGCGGAAGCGGAGGUGGAGGAUCAGGGCCGGCCGGAGGAGGUGGAAGCGGAGGAGGAGGAAGCGGAGGAGG |
| | AGGUAGCGGACCUAAGAAAAAGAGGAAGGUGGCGGCCGCUGGAUCCCCUUCAGGGCAGAUCAGCAACCAGGCCCUGGCUCUGGCCCCUAGC |
| | UCCGCUCCAGUGCUGGCCCAGACUAUGGUGCCCUCUAGUGCUAUGGUGCCUCUGGCCCAGCCACCUGCUCCAGCCCCUGUGCUGACCCCAG |
| | GACCACCCCAGUCACUGAGCGCUCCAGUGCCCAAGUCUACACAGGCCGGCGAGGGGACUCUGAGUGAAGCUCUGCUGCACCUGCAGUUCGA |
| | CGCUGAUGAGGACCUGGGAGCUCUGCUGGGGAACAGCACCGAUCCCGGAGUGUUCACAGAUCUGGCCUCCGUGGACAACUCUGAGUUUCA |
| | GCAGCUGCUGAAUCAGGGCGUGUCCAUGUCUCAUAGUACAGCCGAACCAAUGCUGAUGGAGUACCCCGAAGCCAUUACCCGGCUGGUGACC |
| | GGCAGCCAGCGGCCCCCCGACCCCGCUCCAACUCCCCUGGGAACCAGCGGCCUGCCUAAUGGGCUGUCCGGAGAUGAAGACUUCUCAAGCAU |
| | CGCUGAUAUGGACUUUAGUGCCCUGCUGUCACAGAUUUCCUCUAGUGGGCAGGGAGGAGGUGGAAGCGGCUUCAGCGUGGACACCAGUG |
| | CCCUGCUGGACCUGUUCAGCCCCUCGGUGACCGUGCCCGACAUGAGCCUGCCUGACCUUGACAGCAGCCUGGCCAGUAUCCAAGAGCUCCUG |
| | UCUCCCCAGGAGCCCCCCAGGCCUCCCGAGGCAGAGAACAGCAGCCCGGAUUCAGGGAAGCAGCUGGUGCACUACACAGCGCAGCCGCUGUU |
| | CCUGCUGGACCCCGGCUCCGUGGACACCGGGAGCAACGACCUGCCGGUGCUGUUUGAGCUGGGAGAGGGCUCCUACUUCUCCGAAGGGGAC |
| | GGCUUCGCCGAGGACCCCACCAUCUCCCUGCUGACAGGCUCGGAGCCUCCCAAAGCCAAGGACCCCACUGUCUCCAUCGAUUGA |
| mTR5 (SEQ ID NO:45) | AUGGACUACAAGGAUCACGACGGUGACUAUAAGGAUCAUGACAUCGACUAUAAGGACGAUGACGAUAAGAUCGAUGGCGGAGGCGGAUCU |
| | 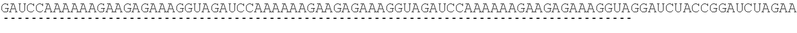 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACUCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAAUC |
| | ACGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACGGUGGCGGCAAGCAAGCGCUCGAAACGGUGCAGCGGCUGUUGCCGGUGCUGUGCCAGGACCAUGGCCUGACUCCGGAC |
| | CAAGUGGUGGCUAUCGCCAGCCACGAUGGCGGCAAGCAAGCGCUCGAAACGGUGCAGCGGCUGUUGCCGGUGCUGUGCCAGGACCAUGG |
| | CCUGACUCCGGACCAAGUGGUGGCUAUCGCCAGCCACGAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACGGUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAAUCACGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAACGGUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAAUCAC |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAACAUUGGCGGCAAGCAAGCGCUCGAAACGGUGCAGCGGCUGUUGCCGGUGCUGUGCCAGGACCAUGGCCUGACCCCGGACCA |
| | AGUGGUGGCUAUCGCCAGCAACGGUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAUUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAACAUUGGCGGCAAGCAAGCGCUCGAAACGGUGCAGCGGCUGUUG |
| | CCGGUGCUGUGCCAGGACCAUGGCCUGACCCCGGACCAAGUGGUGGCUAUCGCCAGCAACAUUGGCGGCAAGCAAGCGCUCGAAACGGUG |
| | CAGCGGCUGUUGCCGGUGCUGUGCCAGGACCAUGGCCUGACCCCGGACCAAGUGGUGGCUAUCGCCAGCAACAUUGGCGGCAAGCAAGCG |
| | CUCGAAACGGUGCAGCGGCUGUUGCCGGUGCUGUGCCAGGACCAUGGCCUGACCCCGGACCAAGUGGUGGCUAUCGCCAGCAACGGUGG |
| | CGGCAAGCAAGCGCUCGAAACGGUGCAGCGGCUGUUGCCGGUGCUGUGCCAGGACCAUGGCCUGACUCCGGACCAAGUGGUGGCUAUCG |
| | CCAGCCACGAUGGCGGCAAGCAAGCGCUCGAAACGGUGCAGCGGCUGUUGCCGGUGCUGUGCCAGGACCAUGGCCUGACCCCGGACCAAG |
| | UGGUGGCUAUCGCCAGCAAUCACGGCGGCAAGCAAGCGCUCGAAACGGUGCAGCGGCUGUUGCCGGUGCUGUGCCAGGACCAUGGCCUG |
| | ACUCCGGACCAAGUGGUGGCUAUCGCCAGCCACGAUGGCGGCAAGCAAGCGCUCGAAACGGUGCAGCGGCUGUUGCCGGUGCUGUGCCA |
| | GGACCAUGGCCUGACCCCGGACCAAGUGGUGGCUAUCGCCAGCAACGGUGGCGGCAAGCAAGCGCUCGAAACGGUGCAGCGGCUGUUGCC |
| | GGUGCUGUGCCAGGACCAUGGCCUGACUCCGGACCAAGUGGUGGCUAUCGCCAGCCACGAUGGCGGCAAGCAAGCGCUCGAAACGGUGCA |
| | GCGGCUGUUGCCGGUGCUGUGCCAGGACCAUGGCCUGACCCCGGACCAAGUGGUGGCUAUCGCCAGCAACAUUGGCGGCAAGCAAGCGCU |
| | CGAAACGGUGCAGCGGCUGUUGCCGGUGCUGUGCCAGGACCAUGGCCUGACCCCGGACCAAGUGGUGGCUAUCGCCAGCAACGGUGGCG |
| | GCAAGCAAGCGCUCGAAACGGUGCAGCGGCUGUUGCCGGUGCUGUGCCAGGACCAUGGCCUGACCCCGGACCAAGUGGUGGCUAUCGCCA |
| | GCAACAUUGGCGGCAAGCAAGCGCUCGAAACGGUGCAGCGGCUGUUGCCGGUGCUGUGCCAGGACCAUGGCCUGACCCCGGACCAAGUG |
| | GUGGCUAUCGCCAGCAAUCACGGCGGCAAGCAAGCGCUCGAAACGGUGCAGCGGCUGUUGCCGGUGCUGUGCCAGGACCAUGGCCUGAC |
| | UCCGGACCAAGUGGUGGCUAUCGCCAGCCACGAUGGCGGCAAGCAAGCGCUCGAAACGGUGCAGCGGCUGUUGCCGGUGCUGUGCCAGG |
| | ACCAUGGCGCUAUCGCCAGCAACGGUGGCGGCAAGCAAGCGCUCGAAAGCAUUGUGGCCCAGCUGAGCCGGCCUGAUCCGGCGUUGGCCG |
| | CGUUAACCAACGACCACCUCGUCGCCUUGGCCUGCCUCGGCGGACGUCCUGCCAUGGAUGCAGUGAAAAAGGGAUUGCCGCACGCGCCGGA |
| | AUUGAUCAGAAGAGUCAAUCGCCGUAUUGGCGAACGCACGUCCCAUCGCGUUGCCAGGGACCCAAAGAAGAAGCGCAAAGUGGAUCCUAAA |
| | AAGAAAAGAAAGGUAGGCGGCCGCGGGGGAGGCGGUUCCGGUGGCGGCGGAAGCGGAGGUGGAGGAUCAGGGCCGGCCGGAGGAGGUGG |
| | AAGCGGAGGAGGAGGAAGCGGAGGAGGAGGUAGCGGACCUAAGAAAAAGAGGAAGGUGGCGGCCGCUGGAUCCCCUUCAGGGCAGAUCAG |
| | CAACCAGGCCCUGGCUCUGGCCCCUAGCUCCGCUCCAGUGCUGGCCCAGACUAUGGUGCCCUCUAGUGCUAUGGUGCCUCUGGCCCAGCCAC |
| | CUGCUCCAGCCCCUGUGCUGACCCCAGGACCACCCCAGUCACUGAGCGCUCCAGUGCCCAAGUCUACACAGGCCGGCGAGGGGACUCUGAGU |
| | GAAGCUCUGCUGCACCUGCAGUUCGACGCUGAUGAGGACCUGGGAGCUCUGCUGGGGAACAGCACCGAUCCCGGAGUGUUCACAGAUCUG |
| | GCCUCCGUGGACAACUCUGAGUUUCAGCAGCUGCUGAAUCAGGGCGUGUCCAUGUCUCAUAGUACAGCCGAACCAAUGCUGAUGGAGUAC |
| | CCCGAAGCCAUUACCCGGCUGGUGACCGGCAGCCAGCGGCCCCCCGACCCCGCUCCAACUCCCCUGGGAACCAGCGGCCUGCCUAAUGGGCUG |
| | UCCGGAGAUGAAGACUUCUCAAGCAUCGCUGAUAUGGACUUUAGUGCCCUGCUGUCACAGAUUUCCUCUAGUGGGCAGGGAGGAGGUGG |
| | AAGCGGCUUCAGCGUGGACACCAGUGCCCUGCUGGACCUGUUCAGCCCCUCGGUGACCGUGCCCGACAUGAGCCUGCCUGACCUUGACAGC |
| | AGCCUGGCCAGUAUCCAAGAGCUCCUGUCUCCCCAGGAGCCCCCCAGGCCUCCCGAGGCAGAGAACAGCAGCCCGGAUUCAGGGAAGCAGCU |
| | GGUGCACUACACAGCGCAGCCGCUGUUCCUGCUGGACCCCGGCUCCGUGGACACCGGGAGCAACGACCUGCCGGUGCUGUUUGAGCUGGGA |
| | GAGGGCUCCUACUUCUCCGAAGGGGACGGCUUCGCCGAGGACCCCACCAUCUCCCUGCUGACAGGCUCGGAGCCUCCCAAAGCCAAGGACCC |
| | CACUGUCUCCAUCGAUUGA |
| mTR6 (SEQ ID NO:46) | AUGGACUACAAGGAUCACGACGGUGACUAUAAGGAUCAUGACAUCGACUAUAAGGACGAUGACGAUAAGAUCGAUGGCGGAGGCGGAUCU |
| | 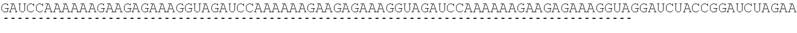 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAAUCACGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAAUCACGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACUCCGGACCAAGUGGUGGCUA |
| | UCGCCAGCCACGAUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAACAUUGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAAUCACGGCGGCAAGCAAGCGCUCGAAACGGUGCAGCGGCUGUUGCCGGUGCUGUGC |
| | CAGGACCAUGGCCUGACUCCGGACCAAGUGGUGGCUAUCGCCAGCCACGAUGGCGGCAAGCAAGCGCUCGAAACGGUGCAGCGGCUGUUG |
| | CCGGUGCUGUGCCAGGACCAUGGCCUGACUCCGGACCAAGUGGUGGCUAUCGCCAGCCACGAUGGCGGCAAGCAAGCGCUCGAAACGGUG |
| | CAGCGGCUGUUGCCGGUGCUGUGCCAGGACCAUGGCCUGACCCCGGACCAAGUGGUGGCUAUCGCCAGCAACAUUGGCGGCAAGCAAGCG |
| | CUCGAAACGGUGCAGCGGCUGUUGCCGGUGCUGUGCCAGGACCAUGGCCUGACCCCGGACCAAGUGGUGGCUAUCGCCAGCAAUCACGGC |
| | GGCAAGCAAGCGCUCGAAACGGUGCAGCGGCUGUUGCCGGUGCUGUGCCAGGACCAUGGCCUGACCCCGGACCAAGUGGUGGCUAUCGCC |
| | AGCAACAUUGGCGGCAAGCAAGCGCUCGAAACGGUGCAGCGGCUGUUGCCGGUGCUGUGCCAGGACCAUGGCCUGACCCCGGACCAAGUG |
| | GUGGCUAUCGCCAGCAACGGUGGCGGCAAGCAAGCGCUCGAAACGGUGCAGCGGCUGUUGCCGGUGCUGUGCCAGGACCAUGGCCUGAC |
| | CCCGGACCAAGUGGUGGCUAUCGCCAGCAAUCACGGCGGCAAGCAAGCGCUCGAAACGGUGCAGCGGCUGUUGCCGGUGCUGUGCCAGG |
| | ACCAUGGCCUGACCCCGGACCAAGUGGUGGCUAUCGCCAGCAACGGUGGCGGCAAGCAAGCGCUCGAAACGGUGCAGCGGCUGUUGCCGG |
| | UGCUGUGCCAGGACCAUGGCGCUAUCGCCAGCAACGGUGGCGGCAAGCAAGCGCUCGAAAGCAUUGUGGCCCAGCUGAGCCGGCCUGAUC |
| | CGGCGUUGGCCGCGUUAACCAACGACCACCUCGUCGCCUUGGCCUGCCUCGGGGGACGUCCUGCCAUGGAUGCAGUGAAAAAGGGAUUGCC |
| | GCACGCGCCGGAAUUGAUCAGAAGAGUCAAUCGCCGUAUUGGCGAACGCACGUCCCAUCGCGUUGCCAGGGACCCAAAGAAGAAGCGCAAA |
| | GUGGAUCCUAAAAAGAAAAGAAAGGUAGGCGGCCGCGGGGGAGGCGGUUCCGGUGGCGGCGGAAGCGGAGGUGGAGGAUCAGGGCCGGC |
| | CGGAGGAGGUGGAAGCGGAGGAGGAGGAAGCGGAGGAGGAGGUAGCGGACCUAAGAAAAAGAGGAAGGUGGCGGCCGCUGGAUCCCCUU |
| | CAGGGCAGAUCAGCAACCAGGCCCUGGCUCUGGCCCCUAGCUCCGCUCCAGUGCUGGCCCAGACUAUGGUGCCCUCUAGUGCUAUGGUGCC |
| | UCUGGCCCAGCCACCUGCUCCAGCCCCUGUGCUGACCCCAGGACCACCCCAGUCACUGAGCGCUCCAGUGCCCAAGUCUACACAGGCCGGCGA |
| | GGGGACUCUGAGUGAAGCUCUGCUGCACCUGCAGUUCGACGCUGAUGAGGACCUGGGAGCUCUGCUGGGGAACAGCACCGAUCCCGGAGU |
| | GUUCACAGAUCUGGCCUCCGUGGACAACUCUGAGUUUCAGCAGCUGCUGAAUCAGGGCGUGUCCAUGUCUCAUAGUACAGCCGAACCAAU |
| | GCUGAUGGAGUACCCCGAAGCCAUUACCCGGCUGGUGACCGGCAGCCAGCGGCCCCCCGACCCCGCUCCAACUCCCCUGGGAACCAGCGGCCU |
| | GCCUAAUGGGCUGUCCGGAGAUGAAGACUUCUCAAGCAUCGCUGAUAUGGACUUUAGUGCCCUGCUGUCACAGAUUUCCUCUAGUGGGCA |
| | GGGAGGAGGUGGAAGCGGCUUCAGCGUGGACACCAGUGCCCUGCUGGACCUGUUCAGCCCCUCGGUGACCGUGCCCGACAUGAGCCUGCCU |
| | GACCUUGACAGCAGCCUGGCCAGUAUCCAAGAGCUCCUGUCUCCCCAGGAGCCCCCCAGGCCUCCCGAGGCAGAGAACAGCAGCCCGGAUUC |
| | AGGGAAGCAGCUGGUGCACUACACAGCGCAGCCGCUGUUCCUGCUGGACCCCGGCUCCGUGGACACCGGGAGCAACGACCUGCCGGUGCUG |
| | UUUGAGCUGGGAGAGGGCUCCUACUUCUCCGAAGGGGACGGCUUCGCCGAGGACCCCACCAUCUCCCUGCUGACAGGCUCGGAGCCUCCCA |
| | AAGCCAAGGACCCCACUGUCUCCAUCGAUUGA |
| mTR7 (SEQ ID NO:47) | AUGGACUACAAGGAUCACGACGGUGACUAUAAGGAUCAUGACAUCGACUAUAAGGACGAUGACGAUAAGAUCGAUGGCGGAGGCGGAUCU |
| | 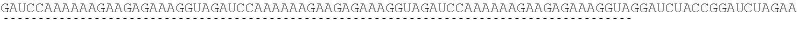 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACUCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAAUCACGGCGGCAAGCAAGCGCUCGAAACGGUGCAGCGGCUGUUGCCGGUGCUGUGCCAGGACCAUGGCCUGACUCCGGAC |
| | CAAGUGGUGGCUAUCGCCAGCCACGAUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAAUCACGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACAUUGGCGGCAAGCAAGCGCUCGAAACGGUGCAGCGGCUGUUGCCGGUGCUGUGCCAGGACCAUGGCCUGACUCCGGACC |
| | AAGUGGUGGCUAUCGCCAGCCACGAUGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAACGGUGGCGGCAAGCAAGCGCUCGAAACGGUGCAGCGGCUGUUGCCGGUGCUGUG |
| | CCAGGACCAUGGCCUGACUCCGGACCAAGUGGUGGCUAUCGCCAGCCACGAUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAUUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACGGUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAACAUUG |
| | GCGGCAAGCAAGCGCUCGAAACGGUGCAGCGGCUGUUGCCGGUGCUGUGCCAGGACCAUGGCCUGACUCCGGACCAAGUGGUGGCUAUC |
| | GCCAGCCACGAUGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAACGGUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACGGUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACAUUGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACAUUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAAUCACGGCGGCAAGCAAGCGC |
| | UCGAAACGGUGCAGCGGCUGUUGCCGGUGCUGUGCCAGGACCAUGGCCUGACUCCGGACCAAGUGGUGGCUAUCGCCAGCCACGAUGGC |
| | GGCAAGCAAGCGCUCGAAACGGUGCAGCGGCUGUUGCCGGUGCUGUGCCAGGACCAUGGCCUGACCCCGGACCAAGUGGUGGCUAUCGCC |
| | AGCAAUCACGGCGGCAAGCAAGCGCUCGAAACGGUGCAGCGGCUGUUGCCGGUGCUGUGCCAGGACCAUGGCCUGACCCCGGACCAAGUG |
| | GUGGCUAUCGCCAGCAACAUUGGCGGCAAGCAAGCGCUCGAAACGGUGCAGCGGCUGUUGCCGGUGCUGUGCCAGGACCAUGGCGCUAU |
| | CGCCAGCAACGGUGGCGGCAAGCAAGCGCUCGAAAGCAUUGUGGCCCAGCUGAGCCGGCCUGAUCCGGCGUUGGCCGCGUUAACCAACGA |
| | CCACCUCGUCGCCUUGGCCUGCCUCGGCGGACGUCCUGCCAUGGAUGCAGUGAAAAAGGGAUUGCCGCACGCGCCGGAAUUGAUCAGAAGA |
| | GUCAAUCGCCGUAUUGGCGAACGCACGUCCCAUCGCGUUGCCAGGGACCCAAAGAAGAAGCGCAAAGUGGAUCCUAAAAAGAAAAGAAAGG |
| | UAGGCGGCCGCGGGGGAGGCGGUUCCGGUGGCGGCGGAAGCGGAGGUGGAGGAUCAGGGCCGGCCGGAGGAGGUGGAAGCGGAGGAGGA |
| | GGAAGCGGAGGAGGAGGUAGCGGACCUAAGAAAAAGAGGAAGGUGGCGGCCGCUGGAUCCCCUUCAGGGCAGAUCAGCAACCAGGCCCUG |
| | GCUCUGGCCCCUAGCUCCGCUCCAGUGCUGGCCCAGACUAUGGUGCCCUCUAGUGCUAUGGUGCCUCUGGCCCAGCCACCUGCUCCAGCCCC |
| | UGUGCUGACCCCAGGACCACCCCAGUCACUGAGCGCUCCAGUGCCCAAGUCUACACAGGCCGGCGAGGGGACUCUGAGUGAAGCUCUGCUG |
| | CACCUGCAGUUCGACGCUGAUGAGGACCUGGGAGCUCUGCUGGGGAACAGCACCGAUCCCGGAGUGUUCACAGAUCUGGCCUCCGUGGACA |
| | ACUCUGAGUUUCAGCAGCUGCUGAAUCAGGGCGUGUCCAUGUCUCAUAGUACAGCCGAACCAAUGCUGAUGGAGUACCCCGAAGCCAUUAC |
| | CCGGCUGGUGACCGGCAGCCAGCGGCCCCCCGACCCCGCUCCAACUCCCCUGGGAACCAGCGGCCUGCCUAAUGGGCUGUCCGGAGAUGAAG |
| | ACUUCUCAAGCAUCGCUGAUAUGGACUUUAGUGCCCUGCUGUCACAGAUUUCCUCUAGUGGGCAGGGAGGAGGUGGAAGCGGCUUCAGCG |
| | UGGACACCAGUGCCCUGCUGGACCUGUUCAGCCCCUCGGUGACCGUGCCCGACAUGAGCCUGCCUGACCUUGACAGCAGCCUGGCCAGUAU |
| | CCAAGAGCUCCUGUCUCCCCAGGAGCCCCCCAGGCCUCCCGAGGCAGAGAACAGCAGCCCGGAUUCAGGGAAGCAGCUGGUGCACUACACAG |
| | CGCAGCCGCUGUUCCUGCUGGACCCCGGCUCCGUGGACACCGGGAGCAACGACCUGCCGGUGCUGUUUGAGCUGGGAGAGGGCUCCUACUU |
| | CUCCGAAGGGGACGGCUUCGCCGAGGACCCCACCAUCUCCCUGCUGACAGGCUCGGAGCCUCCCAAAGCCAAGGACCCCACUGUCUCCAUCG |
| | AUUGA |
| mTR8 (SEQ ID NO:48) | AUGGACUACAAGGAUCACGACGGUGACUAUAAGGAUCAUGACAUCGACUAUAAGGACGAUGACGAUAAGAUCGAUGGCGGAGGCGGAUCU |
| | 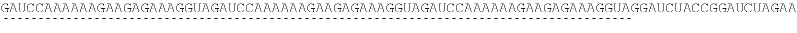 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACUCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAAUCACGGCGGCAAGCAAGCGCUCGAAACGGUGCAGCGGCUGUUGCCGGUGCUGUGCCAGGACCAUGGCCUGACUCCGGAC |
| | CAAGUGGUGGCUAUCGCCAGCCACGAUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAAUCACGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACAUUGGCGGCAAGCAAGCGCUCGAAACGGUGCAGCGGCUGUUGCCGGUGCUGUGCCAGGACCAUGGCCUGACUCCGGACC |
| | AAGUGGUGGCUAUCGCCAGCCACGAUGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAACGGUGGCGGCAAGCAAGCGCUCGAAACGGUGCAGCGGCUGUUGCCGGUGCUGUG |
| | CCAGGACCAUGGCCUGACUCCGGACCAAGUGGUGGCUAUCGCCAGCCACGAUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAUUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACGGUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAACAUUG |
| | GCGGCAAGCAAGCGCUCGAAACGGUGCAGCGGCUGUUGCCGGUGCUGUGCCAGGACCAUGGCCUGACUCCGGACCAAGUGGUGGCUAUC |
| | GCCAGCCACGAUGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAACGGUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACGGUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACAUUGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACAUUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAAUCACGGCGGCAAGCAAGCGC |
| | UCGAAACGGUGCAGCGGCUGUUGCCGGUGCUGUGCCAGGACCAUGGCCUGACUCCGGACCAAGUGGUGGCUAUCGCCAGCCACGAUGGC |
| | GGCAAGCAAGCGCUCGAAACGGUGCAGCGGCUGUUGCCGGUGCUGUGCCAGGACCAUGGCCUGACCCCGGACCAAGUGGUGGCUAUCGCC |
| | AGCAAUCACGGCGGCAAGCAAGCGCUCGAAACGGUGCAGCGGCUGUUGCCGGUGCUGUGCCAGGACCAUGGCCUGACCCCGGACCAAGUG |
| | GUGGCUAUCGCCAGCAACAUUGGCGGCAAGCAAGCGCUCGAAACGGUGCAGCGGCUGUUGCCGGUGCUGUGCCAGGACCAUGGCGCUAU |
| | CGCCAGCAACGGUGGCGGCAAGCAAGCGCUCGAAAGCAUUGUGGCCCAGCUGAGCCGGCCUGAUCCGGCGUUGGCCGCGUUAACCAACGA |
| | CCACCUCGUCGCCUUGGCCUGCCUCGGCGGACGUCCUGCCAUGGAUGCAGUGAAAAAGGGAUUGCCGCACGCGCCGGAAUUGAUCAGAAGA |
| | GUCAAUCGCCGUAUUGGCGAACGCACGUCCCAUCGCGUUGCCAGGGACCCAAAGAAGAAGCGCAAAGUGGAUCCUAAAAAGAAAAGAAAGG |
| | UAGGCGGCCGCGGGGGAGGCGGUUCCGGUGGCGGCGGAAGCGGAGGUGGAGGAUCAGGGCCGGCCGGAGGAGGUGGAAGCGGAGGAGGA |
| | GGAAGCGGAGGAGGAGGUAGCGGACCUAAGAAAAAGAGGAAGGUGGCGGCCGCUGGAUCCCCUUCAGGGCAGAUCAGCAACCAGGCCCUG |
| | GCUCUGGCCCCUAGCUCCGCUCCAGUGCUGGCCCAGACUAUGGUGCCCUCUAGUGCUAUGGUGCCUCUGGCCCAGCCACCUGCUCCAGCCCC |
| | UGUGCUGACCCCAGGACCACCCCAGUCACUGAGCGCUCCAGUGCCCAAGUCUACACAGGCCGGCGAGGGGACUCUGAGUGAAGCUCUGCUG |
| | CACCUGCAGUUCGACGCUGAUGAGGACCUGGGAGCUCUGCUGGGGAACAGCACCGAUCCCGGAGUGUUCACAGAUCUGGCCUCCGUGGACA |
| | ACUCUGAGUUUCAGCAGCUGCUGAAUCAGGGCGUGUCCAUGUCUCAUAGUACAGCCGAACCAAUGCUGAUGGAGUACCCCGAAGCCAUUAC |
| | CCGGCUGGUGACCGGCAGCCAGCGGCCCCCCGACCCCGCUCCAACUCCCCUGGGAACCAGCGGCCUGCCUAAUGGGCUGUCCGGAGAUGAAG |
| | ACUUCUCAAGCAUCGCUGAUAUGGACUUUAGUGCCCUGCUGUCACAGAUUUCCUCUAGUGGGCAGGGAGGAGGUGGAAGCGGCUUCAGCG |
| | UGGACACCAGUGCCCUGCUGGACCUGUUCAGCCCCUCGGUGACCGUGCCCGACAUGAGCCUGCCUGACCUUGACAGCAGCCUGGCCAGUAU |
| | CCAAGAGCUCCUGUCUCCCCAGGAGCCCCCCAGGCCUCCCGAGGCAGAGAACAGCAGCCCGGAUUCAGGGAAGCAGCUGGUGCACUACACAG |
| | CGCAGCCGCUGUUCCUGCUGGACCCCGGCUCCGUGGACACCGGGAGCAACGACCUGCCGGUGCUGUUUGAGCUGGGAGAGGGCUCCUACUU |
| | CUCCGAAGGGGACGGCUUCGCCGAGGACCCCACCAUCUCCCUGCUGACAGGCUCGGAGCCUCCCAAAGCCAAGGACCCCACUGUCUCCAUCG |
| | AUUGA |
| mTR9 (SEQ ID NO:49) | AUGGACUACAAGGAUCACGACGGUGACUAUAAGGAUCAUGACAUCGACUAUAAGGACGAUGACGAUAAGAUCGAUGGCGGAGGCGGAUCU |
| | 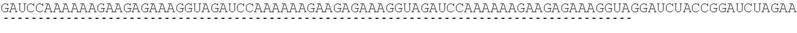 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACUCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAAUCACGGCGGCAAGCA |
| | AGCGCUCGAAACGGUGCAGCGGCUGUUGCCGGUGCUGUGCCAGGACCAUGGCCUGACCCCGGACCAAGUGGUGGCUAUCGCCAGCAAUCA |
| | CGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAAUCACGGCGGCAAGCAAGCGCUCGAAACGGUGCAGCGGCUGUUGCCGGUGCUGUGCCAGGACCAUGGCCUGACUCCGGACC |
| | AAGUGGUGGCUAUCGCCAGCCACGAUGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAACGGUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACGGUGGCGGCAAGCAAGCGCUCGAAACGGUGCAGCGGCUGUU |
| | GCCGGUGCUGUGCCAGGACCAUGGCCUGACUCCGGACCAAGUGGUGGCUAUCGCCAGCCACGAUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACAUUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAAUCACGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAAUCACGGCGGCAAGCAAGCGCUCGAAACGGUGCAGCGGCUGUUGCCGGUGCUGUGCCAGGACCAUGGCCUGACCCCGGACCAAGU |
| | GGUGGCUAUCGCCAGCAACAUUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAAUCACGGCGGCAAGCAAGCGCUCGAAACGGUGCAGCGGCUGUUGCCGGUGCUGUGCCAGG |
| | ACCAUGGCCUGACCCCGGACCAAGUGGUGGCUAUCGCCAGCAACGGUGGCGGCAAGCAAGCGCUCGAAACGGUGCAGCGGCUGUUGCCGG |
| | UGCUGUGCCAGGACCAUGGCCUGACCCCGGACCAAGUGGUGGCUAUCGCCAGCAAUCACGGCGGCAAGCAAGCGCUCGAAACGGUGCAGC |
| | GGCUGUUGCCGGUGCUGUGCCAGGACCAUGGCCUGACCCCGGACCAAGUGGUGGCUAUCGCCAGCAAUCACGGCGGCAAGCAAGCGCUCG |
| | AAACGGUGCAGCGGCUGUUGCCGGUGCUGUGCCAGGACCAUGGCGCUAUCGCCAGCAACGGUGGCGGCAAGCAAGCGCUCGAAAGCAUU |
| | GUGGCCCAGCUGAGCCGGCCUGAUCCGGCGUUGGCCGCGUUAACCAACGACCACCUCGUCGCCUUGGCCUGCCUCGGCGGACGUCCUGCCA |
| | UGGAUGCAGUGAAAAAGGGAUUGCCGCACGCGCCGGAAUUGAUCAGAAGAGUCAAUCGCCGUAUUGGCGAACGCACGUCCCAUCGCGUUG |
| | CCAGGGACCCAAAGAAGAAGCGCAAAGUGGAUCCUAAAAAGAAAAGAAAGGUAGGCGGCCGCGGGGGAGGCGGUUCCGGUGGCGGCGGAA |
| | GCGGAGGUGGAGGAUCAGGGCCGGCCGGAGGAGGUGGAAGCGGAGGAGGAGGAAGCGGAGGAGGAGGUAGCGGACCUAAGAAAAAGAGG |
| | AAGGUGGCGGCCGCUGGAUCCCCUUCAGGGCAGAUCAGCAACCAGGCCCUGGCUCUGGCCCCUAGCUCCGCUCCAGUGCUGGCCCAGACUA |
| | UGGUGCCCUCUAGUGCUAUGGUGCCUCUGGCCCAGCCACCUGCUCCAGCCCCUGUGCUGACCCCAGGACCACCCCAGUCACUGAGCGCUCCA |
| | GUGCCCAAGUCUACACAGGCCGGCGAGGGGACUCUGAGUGAAGCUCUGCUGCACCUGCAGUUCGACGCUGAUGAGGACCUGGGAGCUCUG |
| | CUGGGGAACAGCACCGAUCCCGGAGUGUUCACAGAUCUGGCCUCCGUGGACAACUCUGAGUUUCAGCAGCUGCUGAAUCAGGGCGUGUCC |
| | AUGUCUCAUAGUACAGCCGAACCAAUGCUGAUGGAGUACCCCGAAGCCAUUACCCGGCUGGUGACCGGCAGCCAGCGGCCCCCCGACCCCGC |
| | UCCAACUCCCCUGGGAACCAGCGGCCUGCCUAAUGGGCUGUCCGGAGAUGAAGACUUCUCAAGCAUCGCUGAUAUGGACUUUAGUGCCCUG |
| | CUGUCACAGAUUUCCUCUAGUGGGCAGGGAGGAGGUGGAAGCGGCUUCAGCGUGGACACCAGUGCCCUGCUGGACCUGUUCAGCCCCUCG |
| | GUGACCGUGCCCGACAUGAGCCUGCCUGACCUUGACAGCAGCCUGGCCAGUAUCCAAGAGCUCCUGUCUCCCCAGGAGCCCCCCAGGCCUCC |
| | CGAGGCAGAGAACAGCAGCCCGGAUUCAGGGAAGCAGCUGGUGCACUACACAGCGCAGCCGCUGUUCCUGCUGGACCCCGGCUCCGUGGAC |
| | ACCGGGAGCAACGACCUGCCGGUGCUGUUUGAGCUGGGAGAGGGCUCCUACUUCUCCGAAGGGGACGGCUUCGCCGAGGACCCCACCAUCU |
| | CCCUGCUGACAGGCUCGGAGCCUCCCAAAGCCAAGGACCCCACUGUCUCCAUCGAUUGA |
| mTR10 (SEQ ID NO:50) | AUGGACUACAAGGAUCACGACGGUGACUAUAAGGAUCAUGACAUCGACUAUAAGGACGAUGACGAUAAGAUCGAUGGCGGAGGCGGAUCU |
| | 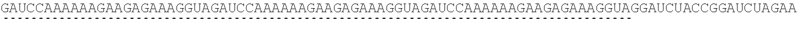 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACGGUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACAUUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAAUCACGGCGGCAAGCA |
| | AGCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACAUUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAAUCACGGCGGCAAGCAAGCGCUCGAAACGGUGCAGCGGCUGUUGCCGGUGCUGUGCCAGGACCAUGGC |
| | CUGACUCCGGACCAAGUGGUGGCUAUCGCCAGCCACGAUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACGGUGGCGGCAAGCAAGCGCUCGAAACGGUGCAGCGGCUGUU |
| | GCCGGUGCUGUGCCAGGACCAUGGCCUGACUCCGGACCAAGUGGUGGCUAUCGCCAGCCACGAUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACGGUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAACAUUG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAACAUUGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAAUCACGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACAUUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACAUUGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACAUUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACAUUGGC |
| | GGCAAGCAAGCGCUCGAAACGGUGCAGCGGCUGUUGCCGGUGCUGUGCCAGGACCAUGGCCUGACCCCGGACCAAGUGGUGGCUAUCGCC |
| | AGCAACAUUGGCGGCAAGCAAGCGCUCGAAACGGUGCAGCGGCUGUUGCCGGUGCUGUGCCAGGACCAUGGCGCUAUCGCCAGCAACGG |
| | UGGCGGCAAGCAAGCGCUCGAAAGCAUUGUGGCCCAGCUGAGCCGGCCUGAUCCGGCGUUGGCCGCGUUAACCAACGACCACCUCGUCGCC |
| | UUGGCCUGCCUCGGCGGACGUCCUGCCAUGGAUGCAGUGAAAAAGGGAUUGCCGCACGCGCCGGAAUUGAUCAGAAGAGUCAAUCGCCGU |
| | AUUGGCGAACGCACGUCCCAUCGCGUUGCCAGGGACCCAAAGAAGAAGCGCAAAGUGGAUCCUAAAAAGAAAAGAAAGGUAGGCGGCCGCG |
| | GGGGAGGCGGUUCCGGUGGCGGCGGAAGCGGAGGUGGAGGAUCAGGGCCGGCCGGAGGAGGUGGAAGCGGAGGAGGAGGAAGCGGAGGA |
| | GGAGGUAGCGGACCUAAGAAAAAGAGGAAGGUGGCGGCCGCUGGAUCCCCUUCAGGGCAGAUCAGCAACCAGGCCCUGGCUCUGGCCCCUA |
| | GCUCCGCUCCAGUGCUGGCCCAGACUAUGGUGCCCUCUAGUGCUAUGGUGCCUCUGGCCCAGCCACCUGCUCCAGCCCCUGUGCUGACCCCA |
| | GGACCACCCCAGUCACUGAGCGCUCCAGUGCCCAAGUCUACACAGGCCGGCGAGGGGACUCUGAGUGAAGCUCUGCUGCACCUGCAGUUCG |
| | ACGCUGAUGAGGACCUGGGAGCUCUGCUGGGGAACAGCACCGAUCCCGGAGUGUUCACAGAUCUGGCCUCCGUGGACAACUCUGAGUUUC |
| | AGCAGCUGCUGAAUCAGGGCGUGUCCAUGUCUCAUAGUACAGCCGAACCAAUGCUGAUGGAGUACCCCGAAGCCAUUACCCGGCUGGUGAC |
| | CGGCAGCCAGCGGCCCCCCGACCCCGCUCCAACUCCCCUGGGAACCAGGGGCCUGCCUAAUGGGCUGUCCGGAGAUGAAGACUUCUCAAGCA |
| | UCGCUGAUAUGGACUUUAGUGCCCUGCUGUCACAGAUUUCCUCUAGUGGGCAGGGAGGAGGUGGAAGCGGCUUCAGCGUGGACACCAGU |
| | GCCCUGCUGGACCUGUUCAGCCCCUCGGUGACCGUGCCCGACAUGAGCCUGCCUGACCUUGACAGCAGCCUGGCCAGUAUCCAAGAGCUCCU |
| | GUCUCCCCAGGAGCCCCCCAGGCCUCCCGAGGCAGAGAACAGCAGCCCGGAUUCAGGGAAGCAGCUGGUGCACUACACAGCGCAGCCGCUGU |
| | UCCUGCUGGACCCCGGCUCCGUGGACACCGGGAGCAACGACCUGCCGGUGCUGUUUGAGCUGGGAGAGGGCUCCUACUUCUCCGAAGGGG |
| | ACGGCUUCGCCGAGGACCCCACCAUCUCCCUGCUGACAGGCUCGGAGCCUCCCAAAGCCAAGGACCCCACUGUCUCCAUCGAUUGA |

TABLE 6
Sequence of synthetic mRNA activating BGLF4

| | |
|---|---|
| mTG1 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO: 51) | 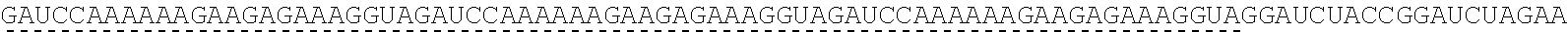 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAAUCACGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACA |
| | UUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAACAUUGGCGGCAAGCAAGCGCUCGAAACGGUGCAGCGGCUGUUGCCGGUGCUGU |
| | GCCAGGACCAUGGCCUGACUCCGGACCAAGUGGUGGCUAUCGCCAGCCACGAUGGCGGCAAGCAAGCGCUCGAAACGGUGCAGCGGCUGU |
| | UGCCGGUGCUGUGCCAGGACCAUGGCCUGACCCCGGACCAAGUGGUGGCUAUCGCCAGCAACAUUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAAUCACGGCGGCAAGCAAG |
| | CGCUCGAAACGGUGCAGCGGCUGUUGCCGGUGCUGUGCCAGGACCAUGGCCUGACUCCGGACCAAGUGGUGGCUAUCGCCAGCCACGAU |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAACGGUGGCGGCAAGCAAGCGCUCGAAACGGUGCAGCGGCUGUUGCCGGUGCUGUGCCAGGACCAUGGCCUGACUCCGGACCA |
| | AGUGGUGGCUAUCGCCAGCCACGAUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACGGUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAAUCACGGCGGCAAGCAAGCGCUCGAAACGGUGCAGCGGCUGUUG |
| | CCGGUGCUGUGCCAGGACCAUGGCCUGACUCCGGACCAAGUGGUGGCUAUCGCCAGCCACGAUGGCGGCAAGCAAGCGCUCGAAACGGUG |
| | CAGCGGCUGUUGCCGGUGCUGUGCCAGGACCAUGGCCUGACUCCGGACCAAGUGGUGGCUAUCGCCAGCCACGAUGGCGGCAAGCAAGCG |
| | CUCGAAACGGUGCAGCGGCUGUUGCCGGUGCUGUGCCAGGACCAUGGCCUGACCCCGGACCAAGUGGUGGCUAUCGCCAGCAACGGUGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAAUCACGGCGGCAAGCAAGCGCUCGAAACGGUGCAGCGGCUGUUGCCGGUGCUGUGCCAGGACCAUGGCCUGACUCCGGACCAAGU |
| | GGUGGCUAUCGCCAGCCACGAUGGCGGCAAGCAAGCGCUCGAAACGGUGCAGCGGCUGUUGCCGGUGCUGUGCCAGGACCAUGGCCUGA |
| | CUCCGGACCAAGUGGUGGCUAUCGCCAGCCACGAUGGCGGCAAGCAAGCGCUCGAAACGGUGCAGCGGCUGUUGCCGGUGCUGUGCCAG |
| | GACCAUGGCCUGACCCCGGACCAAGUGGUGGCUAUCGCCAGCAACGGUGGCGGCAAGCAAGCGCUCGAAACGGUGCAGCGGCUGUUGCCG |
| | GUGCUGUGCCAGGACCAUGGCCUGACCCCGGACCAAGUGGUGGCUAUCGCCAGCAAUCACGGCGGCAAGCAAGCGCUCGAAACGGUGCAG |
| | CGGCUGUUGCCGGUGCUGUGCCAGGACCAUGGCCUGACUCCGGACCAAGUGGUGGCUAUCGCCAGCCACGAUGGCGGCAAGCAAGCGCUC |
| | GAAACGGUGCAGCGGCUGUUGCCGGUGCUGUGCCAGGACCAUGGCCUGACCCCGGACCAAGUGGUGGCUAUCGCCAGCAACGGUGGCGG |
| | CAAGCAAGCGCUCGAAACGGUGCAGCGGCUGUUGCCGGUGCUGUGCCAGGACCAUGGCCUGACCCCGGACCAAGUGGUGGCUAUCGCCAG |
| | CAAUCACGGCGGCAAGCAAGCGCUCGAAACGGUGCAGCGGCUGUUGCCGGUGCUGUGCCAGGACCAUGGCCUGACUCCGGACCAAGUGG |
| | UGGCUAUCGCCAGCCACGAUGGCGGCAAGCAAGCGCUCGAAACGGUGCAGCGGCUGUUGCCGGUGCUGUGCCAGGACCAUGGCCUGACUC |
| | CGGACCAAGUGGUGGCUAUCGCCAGCCACGAUGGCGGCAAGCAAGCGCUCGAAACGGUGCAGCGGCUGUUGCCGGUGCUGUGCCAGGAC |
| | CAUGGCGCUAUCGCCAGCAACGGUGGCGGCAAGCAAGCGCUCGAAAGCAUUGUGGCCCAGCUGAGCCGGCCUGAUCCGGCGUUGGCCGCG |
| | UUAACCAACGACCACCUCGUCGCCUUGGCCUGCCUCGGCGGACGUCCUGCCAUGGAUGCAGUGAAAAAGGGAUUGCCGCACGCGCCGGAAU |
| | UGAUCAGAAGAGUCAAUCGCCGUAUUGGCGAACGCACGUCCCAUCGCGUUGCCAGGGACCCAAAGAAGAAGCGCAAAGUGGAUCCUAAAAA |
| | GAAAAGAAAGGUAGGCGGCCGCGGGGGAGGCGGUUCCGGUGGCGGCGGAAGCGGAGGUGGAGGAUCAGGGCCGGCCGGAGGAGGUGGAA |
| | GCGGAGGAGGAGGAAGCGGAGGAGGAGGUAGCGGACCUAAGAAAAAGAGGAAGGUGGCGGCCGCUGGAUCCCCUUCAGGGCAGAUCAGCA |
| | ACCAGGCCCUGGCUCUGGCCCCUAGCUCCGCUCCAGUGCUGGCCCAGACUAUGGUGCCCUCUAGUGCUAUGGUGCCUCUGGCCCAGCCACC |
| | UGCUCCAGCCCCUGUGCUGACCCCAGGACCACCCCAGUCACUGAGCGCUCCAGUGCCCAAGUCUACACAGGCCGGCGAGGGGACUCUGAGUG |
| | AAGCUCUGCUGCACCUGCAGUUCGACGCUGAUGAGGACCUGGGAGCUCUGCUGGGGAACAGCACCGAUCCCGGAGUGUUCACAGAUCUGG |
| | CCUCCGUGGACAACUCUGAGUUUCAGCAGCUGCUGAAUCAGGGCGUGUCCAUGUCUCAUAGUACAGCCGAACCAAUGCUGAUGGAGUACCC |
| | CGAAGCCAUUACCCGGCUGGUGACCGGCAGCCAGCGGCCCCCCGACCCCGCUCCAACUCCCCUGGGAACCAGCGGCCUGCCUAAUGGGCUGU |
| | CCGGAGAUGAAGACUUCUCAAGCAUCGCUGAUAUGGACUUUAGUGCCCUGCUGUCACAGAUUUCCUCUAGUGGGCAGGGAGGAGGUGGAA |
| | GCGGCUUCAGCGUGGACACCAGUGCCCUGCUGGACCUGUUCAGCCCCUCGGUGACCGUGCCCGACAUGAGCCUGCCUGACCUUGACAGCAG |
| | CCUGGCCAGUAUCCAAGAGCUCCUGUCUCCCCAGGAGCCCCCCAGGCCUCCCGAGGCAGAGAACAGCAGCCCGGAUUCAGGGAAGCAGCUGG |
| | UGCACUACACAGCGCAGCCGCUGUUCCUGCUGGACCCCGGCUCCGUGGACACCGGGAGCAACGACCUGCCGGUGCUGUUUGAGCUGGGAGA |
| | GGGCUCCUACUUCUCCGAAGGGGACGGCUUCGCCGAGGACCCCACCAUCUCCCUGCUGACAGGCUCGGAGCCUCCCAAAGCCAAGGACCCCA |
| | CUGUCUCCAUCGAUUGA |
| mTG2 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:52) | 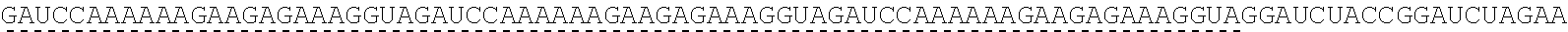 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACUCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAAUCACGGCGGCAAGCA |
| | AGCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACUCCGGACCAAGUGGUGGCUA |
| | UCGCCAGCCACGAUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAACGGUGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAAUCACGGCGGCAAGCAAGCGCUCGAAACGGUGCAGCGGCUGUUGCCGGUGCUGUGC |
| | CAGGACCAUGGCCUGACUCCGGACCAAGUGGUGGCUAUCGCCAGCCACGAUGGCGGCAAGCAAGCGCUCGAAACGGUGCAGCGGCUGUUG |
| | CCGGUGCUGUGCCAGGACCAUGGCCUGACCCCGGACCAAGUGGUGGCUAUCGCCAGCAACGGUGGCGGCAAGCAAGCGCUCGAAACGGUG |
| | CAGCGGCUGUUGCCGGUGCUGUGCCAGGACCAUGGCCUGACCCCGGACCAAGUGGUGGCUAUCGCCAGCAAUCACGGCGGCAAGCAAGCG |
| | CUCGAAACGGUGCAGCGGCUGUUGCCGGUGCUGUGCCAGGACCAUGGCCUGACUCCGGACCAAGUGGUGGCUAUCGCCAGCCACGAUGG |
| | CGGCAAGCAAGCGCUCGAAACGGUGCAGCGGCUGUUGCCGGUGCUGUGCCAGGACCAUGGCCUGACUCCGGACCAAGUGGUGGCUAUCG |
| | CCAGCCACGAUGGCGGCAAGCAAGCGCUCGAAACGGUGCAGCGGCUGUUGCCGGUGCUGUGCCAGGACCAUGGCCUGACCCCGGACCAAG |
| | UGGUGGCUAUCGCCAGCAACGGUGGCGGCAAGCAAGCGCUCGAAACGGUGCAGCGGCUGUUGCCGGUGCUGUGCCAGGACCAUGGCCUG |
| | ACUCCGGACCAAGUGGUGGCUAUCGCCAGCCACGAUGGCGGCAAGCAAGCGCUCGAAACGGUGCAGCGGCUGUUGCCGGUGCUGUGCCA |
| | GGACCAUGGCCUGACCCCGGACCAAGUGGUGGCUAUCGCCAGCAACAUUGGCGGCAAGCAAGCGCUCGAAACGGUGCAGCGGCUGUUGCC |
| | GGUGCUGUGCCAGGACCAUGGCCUGACCCCGGACCAAGUGGUGGCUAUCGCCAGCAAUCACGGCGGCAAGCAAGCGCUCGAAACGGUGCA |
| | GCGGCUGUUGCCGGUGCUGUGCCAGGACCAUGGCCUGACCCCGGACCAAGUGGUGGCUAUCGCCAGCAAUCACGGCGGCAAGCAAGCGCU |
| | CGAAACGGUGCAGCGGCUGUUGCCGGUGCUGUGCCAGGACCAUGGCCUGACCCCGGACCAAGUGGUGGCUAUCGCCAGCAACAUUGGCG |
| | GCAAGCAAGCGCUCGAAACGGUGCAGCGGCUGUUGCCGGUGCUGUGCCAGGACCAUGGCCUGACCCCGGACCAAGUGGUGGCUAUCGCCA |
| | GCAACAUUGGCGGCAAGCAAGCGCUCGAAACGGUGCAGCGGCUGUUGCCGGUGCUGUGCCAGGACCAUGGCCUGACUCCGGACCAAGUG |
| | GUGGCUAUCGCCAGCCACGAUGGCGGCAAGCAAGCGCUCGAAACGGUGCAGCGGCUGUUGCCGGUGCUGUGCCAGGACCAUGGCCUGAC |
| | UCCGGACCAAGUGGUGGCUAUCGCCAGCCACGAUGGCGGCAAGCAAGCGCUCGAAACGGUGCAGCGGCUGUUGCCGGUGCUGUGCCAGG |
| | ACCAUGGCCUGACUCCGGACCAAGUGGUGGCUAUCGCCAGCCACGAUGGCGGCAAGCAAGCGCUCGAAACGGUGCAGCGGCUGUUGCCGG |
| | UGCUGUGCCAGGACCAUGGCCUGACCCCGGACCAAGUGGUGGCUAUCGCCAGCAACGGUGGCGGCAAGCAAGCGCUCGAAACGGUGCAGC |
| | GGCUGUUGCCGGUGCUGUGCCAGGACCAUGGCCUGACCCCGGACCAAGUGGUGGCUAUCGCCAGCAACGGUGGCGGCAAGCAAGCGCUC |
| | GAAACGGUGCAGCGGCUGUUGCCGGUGCUGUGCCAGGACCAUGGCGCUAUCGCCAGCAACGGUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |
| mTG3 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:53) | 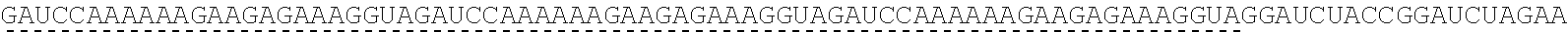 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACUCCGGACCAAGUGGUGGCUAUCGCCAGCCACGAUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACUCCGGACCAAGUGGUGGCU |
| | AUCGCCAGCCACGAUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACCCCGGACCAAGUGGUGGCUAUCGCCAGCAAUCACGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAAUCACGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAUUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACAUUGGCGGCAAGCAAGC |
| | GCUCGAAACGGUGCAGCGGCUGUUGCCGGUGCUGUGCCAGGACCAUGGCCUGACUCCGGACCAAGUGGUGGCUAUCGCCAGCCACGAUG |
| | GCGGCAAGCAAGCGCUCGAAACGGUGCAGCGGCUGUUGCCGGUGCUGUGCCAGGACCAUGGCCUGACUCCGGACCAAGUGGUGGCUAUC |
| | GCCAGCCACGAUGGCGGCAAGCAAGCGCUCGAAACGGUGCAGCGGCUGUUGCCGGUGCUGUGCCAGGACCAUGGCCUGACUCCGGACCAA |
| | GUGGUGGCUAUCGCCAGCCACGAUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAACGGUGGCGGCAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAACGGUGGCGGCAAGCAAGCGCUCGAAACGGUGCAGCGGCUGUUGC |
| | CGGUGCUGUGCCAGGACCAUGGCCUGACCCCGGACCAAGUGGUGGCUAUCGCCAGCAACGGUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAAUCACGGCG |
| | GCAAGCAAGCGCUCGAAACGGUGCAGCGGCUGUUGCCGGUGCUGUGCCAGGACCAUGGCCUGACUCCGGACCAAGUGGUGGCUAUCGCC |
| | AGCCACGAUGGCGGCAAGCAAGCGCUCGAAACGGUGCAGCGGCUGUUGCCGGUGCUGUGCCAGGACCAUGGCCUGACCCCGGACCAAGUG |
| | GUGGCUAUCGCCAGCAACAUUGGCGGCAAGCAAGCGCUCGAAACGGUGCAGCGGCUGUUGCCGGUGCUGUGCCAGGACCAUGGCCUGAC |
| | CCCGGACCAAGUGGUGGCUAUCGCCAGCAACGGUGGCGGCAAGCAAGCGCUCGAAACGGUGCAGCGGCUGUUGCCGGUGCUGUGCCAGG |
| | ACCAUGGCCUGACUCCGGACCAAGUGGUGGCUAUCGCCAGCCACGAUGGCGGCAAGCAAGCGCUCGAAACGGUGCAGCGGCUGUUGCCGG |
| | UGCUGUGCCAGGACCAUGGCCUGACCCCGGACCAAGUGGUGGCUAUCGCCAGCAACAUUGGCGGCAAGCAAGCGCUCGAAACGGUGCAGC |
| | GGCUGUUGCCGGUGCUGUGCCAGGACCAUGGCCUGACUCCGGACCAAGUGGUGGCUAUCGCCAGCCACGAUGGCGGCAAGCAAGCGCUC |
| | GAAACGGUGCAGCGGCUGUUGCCGGUGCUGUGCCAGGACCAUGGCGCUAUCGCCAGCAACGGUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |
| mTG4 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:54) | 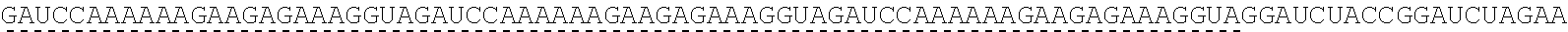 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAUUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACUCCGGACCAAGUGGUGGCU |
| | AUCGCCAGCCACGAUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACUCCGGACCAAGUGGUGGCUAUCGCCAGCCACGAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACGGUGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAAUCACGGCGGCAAGCAAG |
| | CGCUCGAAACGGUGCAGCGGCUGUUGCCGGUGCUGUGCCAGGACCAUGGCCUGACCCCGGACCAAGUGGUGGCUAUCGCCAGCAACGGU |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAAUCACGGCGGCAAGCAAGCGCUCGAAACGGUGCAGCGGCUGUUGCCGGUGCUGUGCCAGGACCAUGGCCUGACCCCGGACCA |
| | AGUGGUGGCUAUCGCCAGCAACGGUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAAUCACGGCGGCAAGCAAGCGCUCGAAACGGUGCAGCGGCUGUUGCCGGUGCUGUGCC |
| | AGGACCAUGGCCUGACUCCGGACCAAGUGGUGGCUAUCGCCAGCCACGAUGGCGGCAAGCAAGCGCUCGAAACGGUGCAGCGGCUGUUGC |
| | CGGUGCUGUGCCAGGACCAUGGCCUGACUCCGGACCAAGUGGUGGCUAUCGCCAGCCACGAUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACGGUGGC |
| | GGCAAGCAAGCGCUCGAAACGGUGCAGCGGCUGUUGCCGGUGCUGUGCCAGGACCAUGGCGCUAUCGCCAGCAACGGUGGCGGCAAGCA |
| | AGCGCUCGAAAGCAUUGUGGCCCAGCUGAGCCGGCCUGAUCCGGCGUUGGCCGCGUUAACCAACGACCACCUCGUCGCCUUGGCCUGCCUC |
| | GGCGGACGUCCUGCCAUGGAUGCAGUGAAAAAGGGAUUGCCGCACGCGCCGGAAUUGAUCAGAAGAGUCAAUCGCCGUAUUGGCGAACGC |
| | ACGUCCCAUCGCGUUGCCAGGGACCCAAAGAAGAAGCGCAAAGUGGAUCCUAAAAAGAAAAGAAAGGUAGGCGGCCGCGGGGGAGGCGGUU |
| | CCGGUGGCGGCGGAAGCGGAGGUGGAGGAUCAGGGCCGGCCGGAGGAGGUGGAAGCGGAGGAGGAGGAAGCGGAGGAGGAGGUAGCGGA |
| | CCUAAGAAAAAGAGGAAGGUGGCGGCCGCUGGAUCCCCUUCAGGGCAGAUCAGCAACCAGGCCCUGGCUCUGGCCCCUAGCUCCGCUCCAG |
| | UGCUGGCCCAGACUAUGGUGCCCUCUAGUGCUAUGGUGCCUCUGGCCCAGCCACCUGCUCCAGCCCCUGUGCUGACCCCAGGACCACCCCAG |
| | UCACUGAGCGCUCCAGUGCCCAAGUCUACACAGGCCGGCGAGGGGACUCUGAGUGAAGCUCUGCUGCACCUGCAGUUCGACGCUGAUGAGG |
| | ACCUGGGAGCUCUGCUGGGGAACAGCACCGAUCCCGGAGUGUUCACAGAUCUGGCCUCCGUGGACAACUCUGAGUUUCAGCAGCUGCUGA |
| | AUCAGGGCGUGUCCAUGUCUCAUAGUACAGCCGAACCAAUGCUGAUGGAGUACCCCGAAGCCAUUACCCGGCUGGUGACCGGCAGCCAGCG |
| | GCCCCCCGACCCCGCUCCAACUCCCCUGGGAACCAGCGGCCUGCCUAAUGGGCUGUCCGGAGAUGAAGACUUCUCAAGCAUCGCUGAUAUGG |
| | ACUUUAGUGCCCUGCUGUCACAGAUUUCCUCUAGUGGGCAGGGAGGAGGUGGAAGCGGCUUCAGCGUGGACACCAGUGCCCUGCUGGACC |
| | UGUUCAGCCCCUCGGUGACCGUGCCCGACAUGAGCCUGCCUGACCUUGACAGCAGCCUGGCCAGUAUCCAAGAGCUCCUGUCUCCCCAGGA |
| | GCCCCCCAGGCCUCCCGAGGCAGAGAACAGCAGCCCGGAUUCAGGGAAGCAGCUGGUGCACUACACAGCGCAGCCGCUGUUCCUGCUGGACC |
| | CCGGCUCCGUGGACACCGGGAGCAACGACCUGCCGGUGCUGUUUGAGCUGGGAGAGGGCUCCUACUUCUCCGAAGGGGACGGCUUCGCCG |
| | AGGACCCCACCAUCUCCCUGCUGACAGGCUCGGAGCCUCCCAAAGCCAAGGACCCCACUGUCUCCAUCGAUUGA |
| mTG5 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:55) | 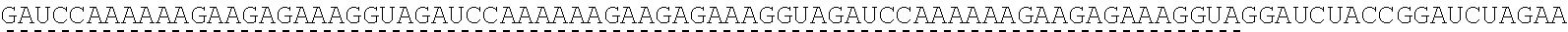 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCAA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACAUUGGCGGCAAGC |
| | AAGCGCUCGAAACGGUGCAGCGGCUGUUGCCGGUGCUGUGCCAGGACCAUGGCCUGACCCCGGACCAAGUGGUGGCUAUCGCCAGCAACG |
| | GUGGCGGCAAGCAAGCGCUCGAAACGGUGCAGCGGCUGUUGCCGGUGCUGUGCCAGGACCAUGGCCUGACUCCGGACCAAGUGGUGGCU |
| | AUCGCCAGCCACGAUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACUCCGGACCAAGUGGUGGCUAUCGCCAGCCACGAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAACGGUGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAAUCACGGCGGCAAGCAAG |
| | CGCUCGAAACGGUGCAGCGGCUGUUGCCGGUGCUGUGCCAGGACCAUGGCCUGACCCCGGACCAAGUGGUGGCUAUCGCCAGCAACGGU |
| | GGCGGCAAGCAAGCGCUCGAAACGGUGCAGCGGCUGUUGCCGGUGCUGUGCCAGGACCAUGGCCUGACCCCGGACCAAGUGGUGGCUAU |
| | CGCCAGCAAUCACGGCGGCAAGCAAGCGCUCGAAACGGUGCAGCGGCUGUUGCCGGUGCUGUGCCAGGACCAUGGCCUGACCCCGGACCA |
| | AGUGGUGGCUAUCGCCAGCAACGGUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAAUCACGGCGGCAAGCAAGCGCUCGAAACGGUGCAGCGGCUGUUGCCGGUGCUGUGCC |
| | AGGACCAUGGCCUGACUCCGGACCAAGUGGUGGCUAUCGCCAGCCACGAUGGCGGCAAGCAAGCGCUCGAAACGGUGCAGCGGCUGUUGC |
| | CGGUGCUGUGCCAGGACCAUGGCCUGACUCCGGACCAAGUGGUGGCUAUCGCCAGCCACGAUGGCGGCAAGCAAGCGCUCGAAACGGUGC |
| | AGCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACGGUGGC |
| | GGCAAGCAAGCGCUCGAAACGGUGCAGCGGCUGUUGCCGGUGCUGUGCCAGGACCAUGGCGCUAUCGCCAGCAACGGUGGCGGCAAGCA |
| | AGCGCUCGAAAGCAUUGUGGCCCAGCUGAGCCGGCCUGAUCCGGCGUUGGCCGCGUUAACCAACGACCACCUCGUCGCCUUGGCCUGCCUC |
| | GGCGGACGUCCUGCCAUGGAUGCAGUGAAAAAGGGAUUGCCGCACGCGCCGGAAUUGAUCAGAAGAGUCAAUCGCCGUAUUGGCGAACGC |
| | ACGUCCCAUCGCGUUGCCAGGGACCCAAAGAAGAAGCGCAAAGUGGAUCCUAAAAAGAAAAGAAAGGUAGGCGGCCGCGGGGGAGGCGGUU |
| | CCGGUGGCGGCGGAAGCGGAGGUGGAGGAUCAGGGCCGGCCGGAGGAGGUGGAAGCGGAGGAGGAGGAAGCGGAGGAGGAGGUAGCGGA |
| | CCUAAGAAAAAGAGGAAGGUGGCGGCCGCUGGAUCCCCUUCAGGGCAGAUCAGCAACCAGGCCCUGGCUCUGGCCCCUAGCUCCGCUCCAG |
| | UGCUGGCCCAGACUAUGGUGCCCUCUAGUGCUAUGGUGCCUCUGGCCCAGCCACCUGCUCCAGCCCCUGUGCUGACCCCAGGACCACCCCAG |
| | UCACUGAGCGCUCCAGUGCCCAAGUCUACACAGGCCGGCGAGGGGACUCUGAGUGAAGCUCUGCUGCACCUGCAGUUCGACGCUGAUGAGG |
| | ACCUGGGAGCUCUGCUGGGGAACAGCACCGAUCCCGGAGUGUUCACAGAUCUGGCCUCCGUGGACAACUCUGAGUUUCAGCAGCUGCUGA |
| | AUCAGGGCGUGUCCAUGUCUCAUAGUACAGCCGAACCAAUGCUGAUGGAGUACCCCGAAGCCAUUACCCGGCUGGUGACCGGCAGCCAGCG |
| | GCCCCCCGACCCCGCUCCAACUCCCCUGGGAACCAGCGGCCUGCCUAAUGGGCUGUCCGGAGAUGAAGACUUCUCAAGCAUCGCUGAUAUGG |
| | ACUUUAGUGCCCUGCUGUCACAGAUUUCCUCUAGUGGGCAGGGAGGAGGUGGAAGCGGCUUCAGCGUGGACACCAGUGCCCUGCUGGACC |
| | UGUUCAGCCCCUCGGUGACCGUGCCCGACAUGAGCCUGCCUGACCUUGACAGCAGCCUGGCCAGUAUCCAAGAGCUCCUGUCUCCCCAGGA |
| | GCCCCCCAGGCCUCCCGAGGCAGAGAACAGCAGCCCGGAUUCAGGGAAGCAGCUGGUGCACUACACAGCGCAGCCGCUGUUCCUGCUGGACC |
| | CCGGCUCCGUGGACACCGGGAGCAACGACCUGCCGGUGCUGUUUGAGCUGGGAGAGGGCUCCUACUUCUCCGAAGGGGACGGCUUCGCCG |
| | AGGACCCCACCAUCUCCCUGCUGACAGGCUCGGAGCCUCCCAAAGCCAAGGACCCCACUGUCUCCAUCGAUUGA |
| mTG6 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:56) | 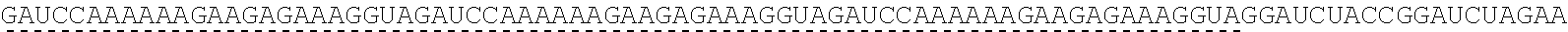 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACUCCGGACCAAGUGGUGGCUAUCGCCAGCCACGAUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACGGUGGCGGCAAGCAAGCGCUCGAAACGGUGCAGCGGCUGUUGCCGGUGCUGUGCCAGGACCAUGGCCUGACCCCGGAC |
| | CAAGUGGUGGCUAUCGCCAGCAACAUUGGCGGCAAGCAAGCGCUCGAAACGGUGCAGCGGCUGUUGCCGGUGCUGUGCCAGGACCAUGG |
| | CCUGACUCCGGACCAAGUGGUGGCUAUCGCCAGCCACGAUGGCGGCAAGCAAGCGCUCGAAACGGUGCAGCGGCUGUUGCCGGUGCUGU |
| | GCCAGGACCAUGGCCUGACUCCGGACCAAGUGGUGGCUAUCGCCAGCCACGAUGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACUCCGGACCAAGUGGUGGCUAUCGCCAGCCACGAUGGCGGCAAGCAA |
| | GCGCUCGAAACGGUGCAGCGGCUGUUGCCGGUGCUGUGCCAGGACCAUGGCCUGACCCCGGACCAAGUGGUGGCUAUCGCCAGCAACAU |
| | UGGCGGCAAGCAAGCGCUCGAAACGGUGCAGCGGCUGUUGCCGGUGCUGUGCCAGGACCAUGGCCUGACUCCGGACCAAGUGGUGGCUA |
| | UCGCCAGCCACGAUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAAUCACGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAACGGUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACAUUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACGGUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAAUCACGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAACAUUG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAACGGUGGCGGCAAGCAAGCGCUCGAAACGGUGCAGCGGCUGUUGCCGGUGCUGUGCCAGGACCAUGGCCUGACUCCGGACCAA |
| | GUGGUGGCUAUCGCCAGCCACGAUGGCGGCAAGCAAGCGCUCGAAACGGUGCAGCGGCUGUUGCCGGUGCUGUGCCAGGACCAUGGCGC |
| | UAUCGCCAGCAACAAUGGCGGCAAGCAAGCGCUCGAAAGCAUUGUGGCCCAGCUGAGCCGGCCUGAUCCGGCGUUGGCCGCGUUAACCAA |
| | CGACCACCUCGUCGCCUUGGCCUGCCUCGGCGGACGUCCUGCCAUGGAUGCAGUGAAAAAGGGAUUGCCGCACGCGCCGGAAUUGAUCAGA |
| | AGAGUCAAUCGCCGUAUUGGCGAACGCACGUCCCAUCGCGUUGCCAGGGACCCAAAGAAGAAGCGCAAAGUGGAUCCUAAAAAGAAAAGAA |
| | AGGUAGGCGGCCGCGGGGGAGGCGGUUCCGGUGGCGGCGGAAGCGGAGGUGGAGGAUCAGGGCCGGCCGGAGGAGGUGGAAGCGGAGGA |
| | GGAGGAAGCGGAGGAGGAGGUAGCGGACCUAAGAAAAAGAGGAAGGUGGCGGCCGCUGGAUCCCCUUCAGGGCAGAUCAGCAACCAGGCC |
| | CUGGCUCUGGCCCCUAGCUCCGCUCCAGUGCUGGCCCAGACUAUGGUGCCCUCUAGUGCUAUGGUGCCUCUGGCCCAGCCACCUGCUCCAG |
| | CCCCUGUGCUGACCCCAGGACCACCCCAGUCACUGAGCGCUCCAGUGCCCAAGUCUACACAGGCCGGCGAGGGGACUCUGAGUGAAGCUCUG |
| | CUGCACCUGCAGUUCGACGCUGAUGAGGACCUGGGAGCUCUGCUGGGGAACAGCACCGAUCCCGGAGUGUUCACAGAUCUGGCCUCCGUG |
| | GACAACUCUGAGUUUCAGCAGCUGCUGAAUCAGGGCGUGUCCAUGUCUCAUAGUACAGCCGAACCAAUGCUGAUGGAGUACCCCGAAGCCA |
| | UUACCCGGCUGGUGACCGGCAGCCAGCGGCCCCCCGACCCCGCUCCAACUCCCCUGGGAACCAGCGGCCUGCCUAAUGGGCUGUCCGGAGAU |
| | GAAGACUUCUCAAGCAUCGCUGAUAUGGACUUUAGUGCCCUGCUGUCACAGAUUUCCUCUAGUGGGCAGGGAGGAGGUGGAAGCGGCUU |
| | CAGCGUGGACACCAGUGCCCUGCUGGACCUGUUCAGCCCCUCGGUGACCGUGCCCGACAUGAGCCUGCCUGACCUUGACAGCAGCCUGGCCA |
| | GUAUCCAAGAGCUCCUGUCUCCCCAGGAGCCCCCCAGGCCUCCCGAGGCAGAGAACAGCAGCCCGGAUUCAGGGAAGCAGCUGGUGCACUAC |
| | ACAGCGCAGCCGCUGUUCCUGCUGGACCCCGGCUCCGUGGACACCGGGAGCAACGACCUGCCGGUGCUGUUUGAGCUGGGAGAGGGCUCCU |
| | ACUUCUCCGAAGGGGACGGCUUCGCCGAGGACCCCACCAUCUCCCUGCUGACAGGCUCGGAGCCUCCCAAAGCCAAGGACCCCACUGUCUCC |
| | AUCGAUUGA |
| mTG7 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:57) | 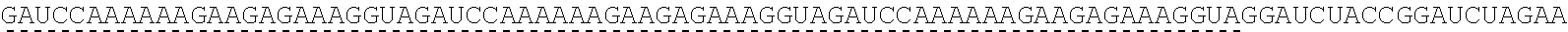 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAAUCACGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAAUCACGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAACGGUGGCGGCAAGC |
| | AAGCGCUCGAAACGGUGCAGCGGCUGUUGCCGGUGCUGUGCCAGGACCAUGGCCUGACUCCGGACCAAGUGGUGGCUAUCGCCAGCCACG |
| | AUGGCGGCAAGCAAGCGCUCGAAACGGUGCAGCGGCUGUUGCCGGUGCUGUGCCAGGACCAUGGCCUGACCCCGGACCAAGUGGUGGCU |
| | AUCGCCAGCAACAUUGGCGGCAAGCAAGCGCUCGAAACGGUGCAGCGGCUGUUGCCGGUGCUGUGCCAGGACCAUGGCCUGACUCCGGAC |
| | CAAGUGGUGGCUAUCGCCAGCCACGAUGGCGGCAAGCAAGCGCUCGAAACGGUGCAGCGGCUGUUGCCGGUGCUGUGCCAGGACCAUGG |
| | CCUGACUCCGGACCAAGUGGUGGCUAUCGCCAGCCACGAUGGCGGCAAGCAAGCGCUCGAAACGGUGCAGCGGCUGUUGCCGGUGCUGU |
| | GCCAGGACCAUGGCCUGACCCCGGACCAAGUGGUGGCUAUCGCCAGCAAUCACGGCGGCAAGCAAGCGCUCGAAACGGUGCAGCGGCUGU |
| | UGCCGGUGCUGUGCCAGGACCAUGGCCUGACUCCGGACCAAGUGGUGGCUAUCGCCAGCCACGAUGGCGGCAAGCAAGCGCUCGAAACGG |
| | UGCAGCGGCUGUUGCCGGUGCUGUGCCAGGACCAUGGCCUGACCCCGGACCAAGUGGUGGCUAUCGCCAGCAAUCACGGCGGCAAGCAAG |
| | CGCUCGAAACGGUGCAGCGGCUGUUGCCGGUGCUGUGCCAGGACCAUGGCCUGACCCCGGACCAAGUGGUGGCUAUCGCCAGCAACAUU |
| | GGCGGCAAGCAAGCGCUCGAAACGGUGCAGCGGCUGUUGCCGGUGCUGUGCCAGGACCAUGGCCUGACUCCGGACCAAGUGGUGGCUAU |
| | CGCCAGCCACGAUGGCGGCAAGCAAGCGCUCGAAACGGUGCAGCGGCUGUUGCCGGUGCUGUGCCAGGACCAUGGCCUGACUCCGGACCA |
| | AGUGGUGGCUAUCGCCAGCCACGAUGGCGGCAAGCAAGCGCUCGAAACGGUGCAGCGGCUGUUGCCGGUGCUGUGCCAGGACCAUGGCC |
| | UGACCCCGGACCAAGUGGUGGCUAUCGCCAGCAACAUUGGCGGCAAGCAAGCGCUCGAAACGGUGCAGCGGCUGUUGCCGGUGCUGUGC |
| | CAGGACCAUGGCCUGACCCCGGACCAAGUGGUGGCUAUCGCCAGCAACAUUGGCGGCAAGCAAGCGCUCGAAACGGUGCAGCGGCUGUUG |
| | CCGGUGCUGUGCCAGGACCAUGGCCUGACCCCGGACCAAGUGGUGGCUAUCGCCAGCAAUCACGGCGGCAAGCAAGCGCUCGAAACGGUG |
| | CAGCGGCUGUUGCCGGUGCUGUGCCAGGACCAUGGCCUGACCCCGGACCAAGUGGUGGCUAUCGCCAGCAACAUUGGCGGCAAGCAAGCG |
| | CUCGAAACGGUGCAGCGGCUGUUGCCGGUGCUGUGCCAGGACCAUGGCCUGACUCCGGACCAAGUGGUGGCUAUCGCCAGCCACGAUGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAACGGUGGCGGCAAGCAAGCGCUCGAAACGGUGCAGCGGCUGUUGCCGGUGCUGUGCCAGGACCAUGGCCUGACUCCGGACCAAG |
| | UGGUGGCUAUCGCCAGCCACGAUGGCGGCAAGCAAGCGCUCGAAACGGUGCAGCGGCUGUUGCCGGUGCUGUGCCAGGACCAUGGCCUG |
| | ACCCCGGACCAAGUGGUGGCUAUCGCCAGCAACAUUGGCGGCAAGCAAGCGCUCGAAACGGUGCAGCGGCUGUUGCCGGUGCUGUGCCA |
| | GGACCAUGGCCUGACCCCGGACCAAGUGGUGGCUAUCGCCAGCAACAUUGGCGGCAAGCAAGCGCUCGAAACGGUGCAGCGGCUGUUGCC |
| | GGUGCUGUGCCAGGACCAUGGCCUGACCCCGGACCAAGUGGUGGCUAUCGCCAGCAACGGUGGCGGCAAGCAAGCGCUCGAAACGGUGCA |
| | GCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCGCUAUCGCCAGCAACAAUGGCGGCAAGCAAGCGCUCGAAAGC |
| | AUUGUGGCCCAGCUGAGCCGGCCUGAUCCGGCGUUGGCCGCGUUAACCAACGACCACCUCGUCGCCUUGGCCUGCCUCGGCGGACGUCCUG |
| | CCAUGGAUGCAGUGAAAAAGGGAUUGCCGCACGCGCCGGAAUUGAUCAGAAGAGUCAAUCGCCGUAUUGGCGAACGCACGUCCCAUCGCGU |
| | UGCCAGGGACCCAAAGAAGAAGCGCAAAGUGGAUCCUAAAAAGAAAAGAAAGGUAGGCGGCCGCGGGGGAGGCGGUUCCGGUGGCGGCGG |
| | AAGCGGAGGUGGAGGAUCAGGGCCGGCCGGAGGAGGUGGAAGCGGAGGAGGAGGAAGCGGAGGAGGAGGUAGCGGACCUAAGAAAAAGA |
| | GGAAGGUGGCGGCCGCUGGAUCCCCUUCAGGGCAGAUCAGCAACCAGGCCCUGGCUCUGGCCCCUAGCUCCGCUCCAGUGCUGGCCCAGAC |
| | UAUGGUGCCCUCUAGUGCUAUGGUGCCUCUGGCCCAGCCACCUGCUCCAGCCCCUGUGCUGACCCCAGGACCACCCCAGUCACUGAGCGCUC |
| | CAGUGCCCAAGUCUACACAGGCCGGCGAGGGGACUCUGAGUGAAGCUCUGCUGCACCUGCAGUUCGACGCUGAUGAGGACCUGGGAGCUC |
| | UGCUGGGGAACAGCACCGAUCCCGGAGUGUUCACAGAUCUGGCCUCCGUGGACAACUCUGAGUUUCAGCAGCUGCUGAAUCAGGGCGUGU |
| | CCAUGUCUCAUAGUACAGCCGAACCAAUGCUGAUGGAGUACCCCGAAGCCAUUACCCGGCUGGUGACCGGCAGCCAGCGGCCCCCCGACCCC |
| | GCUCCAACUCCCCUGGGAACCAGCGGCCUGCCUAAUGGGCUGUCCGGAGAUGAAGACUUCUCAAGCAUCGCUGAUAUGGACUUUAGUGCCC |
| | UGCUGUCACAGAUUUCCUCUAGUGGGCAGGGAGGAGGUGGAAGCGGCUUCAGCGUGGACACCAGUGCCCUGCUGGACCUGUUCAGCCCCU |
| | CGGUGACCGUGCCCGACAUGAGCCUGCCUGACCUUGACAGCAGCCUGGCCAGUAUCCAAGAGCUCCUGUCUCCCCAGGAGCCCCCCAGGCCU |
| | CCCGAGGCAGAGAACAGCAGCCCGGAUUCAGGGAAGCAGCUGGUGCACUACACAGCGCAGCCGCUGUUCCUGCUGGACCCCGGCUCCGUGG |
| | ACACCGGGAGCAACGACCUGCCGGUGCUGUUUGAGCUGGGAGAGGGCUCCUACUUCUCCGAAGGGGACGGCUUCGCCGAGGACCCCACCAU |
| | CUCCCUGCUGACAGGCUCGGAGCCUCCCAAAGCCAAGGACCCCACUGUCUCCAUCGAUUGA |
| mTG8 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:58) | 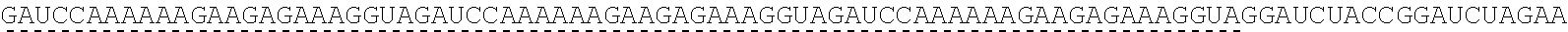 |
| | ACGAUGGUGGUGGUGGAAGGGGGGGGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACGGUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAAUCACGGCGGCAAGCA |
| | AGCGCUCGAAACGGUGCAGCGGCUGUUGCCGGUGCUGUGCCAGGACCAUGGCCUGACUCCGGACCAAGUGGUGGCUAUCGCCAGCCACGA |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACAUUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAACGGUGGCGGCAAGCAAGCGCUCGAAACGGUGCAGCGGCUGUUGCCGGUGCUGUGCCAGGACCAUGGC |
| | CUGACUCCGGACCAAGUGGUGGCUAUCGCCAGCCACGAUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACAUUGGCGGCAAGCAAGCGCUCGAAACGGUGCAGCGGCUGUU |
| | GCCGGUGCUGUGCCAGGACCAUGGCCUGACUCCGGACCAAGUGGUGGCUAUCGCCAGCCACGAUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAACGGUGGCGGCAAGCAAGC |
| | GCUCGAAACGGUGCAGCGGCUGUUGCCGGUGCUGUGCCAGGACCAUGGCCUGACUCCGGACCAAGUGGUGGCUAUCGCCAGCCACGAUG |
| | GCGGCAAGCAAGCGCUCGAAACGGUGCAGCGGCUGUUGCCGGUGCUGUGCCAGGACCAUGGCCUGACCCCGGACCAAGUGGUGGCUAUC |
| | GCCAGCAAUCACGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAACGGUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAAUCACGGCGGCAAGCAAGCGCUCGAAACGGUGCAGCGGCUGUUGCCGGUGCUGUGCCA |
| | GGACCAUGGCGCUAUCGCCAGCAACAAUGGCGGCAAGCAAGCGCUCGAAAGCAUUGUGGCCCAGCUGAGCCGGCCUGAUCCGGCGUUGGC |
| | CGCGUUAACCAACGACCACCUCGUCGCCUUGGCCUGCCUCGGCGGACGUCCUGCCAUGGAUGCAGUGAAAAAGGGAUUGCCGCACGCGCCG |
| | GAAUUGAUCAGAAGAGUCAAUCGCCGUAUUGGCGAACGCACGUCCCAUCGCGUUGCCAGGGACCCAAAGAAGAAGCGCAAAGUGGAUCCUA |
| | AAAAGAAAAGAAAGGUAGGCGGCCGCGGGGGAGGCGGUUCCGGUGGCGGCGGAAGCGGAGGUGGAGGAUCAGGGCCGGCCGGAGGAGGU |
| | GGAAGCGGAGGAGGAGGAAGCGGAGGAGGAGGUAGCGGACCUAAGAAAAAGAGGAAGGUGGCGGCCGCUGGAUCCCCUUCAGGGCAGAUC |
| | AGCAACCAGGCCCUGGCUCUGGCCCCUAGCUCCGCUCCAGUGCUGGCCCAGACUAUGGUGCCCUCUAGUGCUAUGGUGCCUCUGGCCCAGC |
| | CACCUGCUCCAGCCCCUGUGCUGACCCCAGGACCACCCCAGUCACUGAGCGCUCCAGUGCCCAAGUCUACACAGGCCGGCGAGGGGACUCUG |
| | AGUGAAGCUCUGCUGCACCUGCAGUUCGACGCUGAUGAGGACCUGGGAGCUCUGCUGGGGAACAGCACCGAUCCCGGAGUGUUCACAGAU |
| | CUGGCCUCCGUGGACAACUCUGAGUUUCAGCAGCUGCUGAAUCAGGGCGUGUCCAUGUCUCAUAGUACAGCCGAACCAAUGCUGAUGGAG |
| | UACCCCGAAGCCAUUACCCGGCUGGUGACCGGCAGCCAGCGGCCCCCCGACCCCGCUCCAACUCCCCUGGGAACCAGCGGCCUGCCUAAUGGG |
| | CUGUCCGGAGAUGAAGACUUCUCAAGCAUCGCUGAUAUGGACUUUAGUGCCCUGCUGUCACAGAUUUCCUCUAGUGGGCAGGGAGGAGG |
| | UGGAAGCGGCUUCAGCGUGGACACCAGUGCCCUGCUGGACCUGUUCAGCCCCUCGGUGACCGUGCCCGACAUGAGCCUGCCUGACCUUGAC |
| | AGCAGCCUGGCCAGUAUCCAAGAGCUCCUGUCUCCCCAGGAGCCCCCCAGGCCUCCCGAGGCAGAGAACAGCAGCCCGGAUUCAGGGAAGCA |
| | GCUGGUGCACUACACAGCGCAGCCGCUGUUCCUGCUGGACCCCGGCUCCGUGGACACCGGGAGCAACGACCUGCCGGUGCUGUUUGAGCUG |
| | GGAGAGGGCUCCUACUUCUCCGAAGGGGACGGCUUCGCCGAGGACCCCACCAUCUCCCUGCUGACAGGCUCGGAGCCUCCCAAAGCCAAGGA |
| | CCCCACUGUCUCCAUCGAUUGA |
| mTG9 (SEQ ID | AUGGACUACAAGGAUCACGACGGUGACUAUAAGGAUCAUGACAUCGACUAUAAGGACGAUGACGAUAAGAUCGAUGGCGGAGGGGAUCU |
| NO:59) | 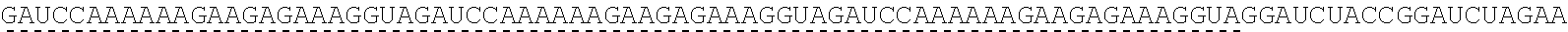 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACCCCGGACCAAGUGGUGGCUAUCGCCAGCAACAUUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACAUUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAAUCACGGCGGCAAGCA |
| | AGCGCUCGAAACGGUGCAGCGGCUGUUGCCGGUGCUGUGCCAGGACCAUGGCCUGACCCCGGACCAAGUGGUGGCUAUCGCCAGCAACAU |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAAUCACGGCGGCAAGCAAGCGCUCGAAACGGUGCAGCGGCUGUUGCCGGUGCUGUGCCAGGACCAUGGCCUGACUCCGGACC |
| | AAGUGGUGGCUAUCGCCAGCCACGAUGGCGGCAAGCAAGCGCUCGAAACGGUGCAGCGGCUGUUGCCGGUGCUGUGCCAGGACCAUGGC |
| | CUGACUCCGGACCAAGUGGUGGCUAUCGCCAGCCACGAUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACGGUGGCGGCAAGCAAGCGCUCGAAACGGUGCAGCGGCUGUU |
| | GCCGGUGCUGUGCCAGGACCAUGGCCUGACUCCGGACCAAGUGGUGGCUAUCGCCAGCCACGAUGGCGGCAAGCAAGCGCUCGAAACGGU |
| | GCAGCGGCUGUUGCCGGUGCUGUGCCAGGACCAUGGCCUGACCCCGGACCAAGUGGUGGCUAUCGCCAGCAAUCACGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAAUCACGG |
| | CGGCAAGCAAGCGCUCGAAACGGUGCAGCGGCUGUUGCCGGUGCUGUGCCAGGACCAUGGCCUGACCCCGGACCAAGUGGUGGCUAUCGC |
| | CAGCAACGGUGGCGGCAAGCAAGCGCUCGAAACGGUGCAGCGGCUGUUGCCGGUGCUGUGCCAGGACCAUGGCCUGACCCCGGACCAAGU |
| | GGUGGCUAUCGCCAGCAACAUUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAAUCACGGCGGCAAGCAAGCGCUCGAAACGGUGCAGCGGCUGUUGCCGGUGCUGUGCCAGG |
| | ACCAUGGCCUGACCCCGGACCAAGUGGUGGCUAUCGCCAGCAACAUUGGCGGCAAGCAAGCGCUCGAAACGGUGCAGCGGCUGUUGCCGG |
| | UGCUGUGCCAGGACCAUGGCCUGACCCCGGACCAAGUGGUGGCUAUCGCCAGCAACAUUGGCGGCAAGCAAGCGCUCGAAACGGUGCAGC |
| | GGCUGUUGCCGGUGCUGUGCCAGGACCAUGGCCUGACCCCGGACCAAGUGGUGGCUAUCGCCAGCAACAUUGGCGGCAAGCAAGCGCUCG |
| | AAACGGUGCAGCGGCUGUUGCCGGUGCUGUGCCAGGACCAUGGCCUGACCCCGGACCAAGUGGUGGCUAUCGCCAGCAACAUUGGCGGC |
| | AAGCAAGCGCUCGAAACGGUGCAGCGGCUGUUGCCGGUGCUGUGCCAGGACCAUGGCGCUAUCGCCAGCAACAAUGGCGGCAAGCAAGC |
| | GCUCGAAAGCAUUGUGGCCCAGCUGAGCCGGCCUGAUCCGGCGUUGGCCGCGUUAACCAACGACCACCUCGUCGCCUUGGCCUGCCUCGGC |
| | GGACGUCCUGCCAUGGAUGCAGUGAAAAAGGGAUUGCCGCACGCGCCGGAAUUGAUCAGAAGAGUCAAUCGCCGUAUUGGCGAACGCACG |
| | UCCCAUCGCGUUGCCAGGGACCCAAAGAAGAAGCGCAAAGUGGAUCCUAAAAAGAAAAGAAAGGUAGGCGGCCGCGGGGGAGGCGGUUCCG |
| | GUGGCGGCGGAAGCGGAGGUGGAGGAUCAGGGCCGGCCGGAGGAGGUGGAAGCGGAGGAGGAGGAAGCGGAGGAGGAGGUAGCGGACCU |
| | AAGAAAAAGAGGAAGGUGGCGGCCGCUGGAUCCCCUUCAGGGCAGAUCAGCAACCAGGCCCUGGCUCUGGCCCCUAGCUCCGCUCCAGUGC |
| | UGGCCCAGACUAUGGUGCCCUCUAGUGCUAUGGUGCCUCUGGCCCAGCCACCUGCUCCAGCCCCUGUGCUGACCCCAGGACCACCCCAGUCA |
| | CUGAGCGCUCCAGUGCCCAAGUCUACACAGGCCGGCGAGGGGACUCUGAGUGAAGCUCUGCUGCACCUGCAGUUCGACGCUGAUGAGGACC |
| | UGGGAGCUCUGCUGGGGAACAGCACCGAUCCCGGAGUGUUCACAGAUCUGGCCUCCGUGGACAACUCUGAGUUUCAGCAGCUGCUGAAUC |
| | AGGGCGUGUCCAUGUCUCAUAGUACAGCCGAACCAAUGCUGAUGGAGUACCCCGAAGCCAUUACCCGGCUGGUGACCGGCAGCCAGCGGCC |
| | CCCCGACCCCGCUCCAACUCCCCUGGGAACCAGCGGCCUGCCUAAUGGGCUGUCCGGAGAUGAAGACUUCUCAAGCAUCGCUGAUAUGGACU |
| | UUAGUGCCCUGCUGUCACAGAUUUCCUCUAGUGGGCAGGGAGGAGGUGGAAGCGGCUUCAGCGUGGACACCAGUGCCCUGCUGGACCUGU |
| | UCAGCCCCUCGGUGACCGUGCCCGACAUGAGCCUGCCUGACCUUGACAGCAGCCUGGCCAGUAUCCAAGAGCUCCUGUCUCCCCAGGAGCCC |
| | CCCAGGCCUCCCGAGGCAGAGAACAGCAGCCCGGAUUCAGGGAAGCAGCUGGUGCACUACACAGCGCAGCCGCUGUUCCUGCUGGACCCCGG |
| | CUCCGUGGACACCGGGAGCAACGACCUGCCGGUGCUGUUUGAGCUGGGAGAGGGCUCCUACUUCUCCGAAGGGGACGGCUUCGCCGAGGA |
| | CCCCACCAUCUCCCUGCUGACAGGCUCGGAGCCUCCCAAAGCCAAGGACCCCACUGUCUCCAUCGAUUGA |
| mTG10 (SEQ ID | AUGGACUACAAGGAUCACGACGGUGACUAUAAGGAUCAUGACAUCGACUAUAAGGACGAUGACGAUAAGAUCGAUGGCGGAGGCGGAUCU |
| NO:60) | 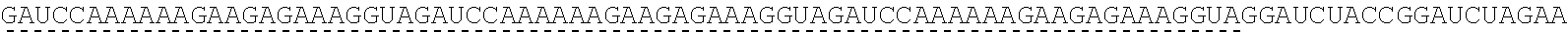 |
| | ACGAUGGUGGUGGUGGAAGCGGGGGUGGGGGCAGCGGUGGAGGGGGAAGCGGGCGCGCCGUGGAUCUACGCACGCUCGGCUACAGUCAG |
| | CAGCAGCAAGAGAAGAUCAAACCGAAGGUGCGUUCGACAGUGGCGCAGCACCACGAGGCACUGGUGGGCCAUGGGUUUACACACGCGCACA |
| | UCGUUGCGCUCAGCCAACACCCGGCAGCGUUAGGGACCGUCGCUGUCACGUAUCAGCACAUAAUCACGGCGUUGCCAGAGGCGACACACGA |
| | AGACAUCGUUGGCGUCGGCAAACAGUGGUCCGGCGCACGCGCCCUGGAGGCCUUGCUCACGGAUGCGGGGGAGUUGAGAGGUCCGCCGUU |
| | ACAGUUGGACACAGGCCAACUUGUGAAGAUUGCAAAACGUGGCGGCGUGACCGCAAUGGAGGCAGUGCAUGCAUCGCGCAAUGCACUGAC |
| | GGGUGCCCCCCUGAACCUGACUCCGGACCAAGUGGUGGCUAUCGCCAGCCACGAUGGCGGCAAGCAAGCGCUCGAAACGGUGCAGCGGCU |
| | GUUGCCGGUGCUGUGCCAGGACCAUGGCCUGACCCCGGACCAAGUGGUGGCUAUCGCCAGCAACGGUGGCGGCAAGCAAGCGCUCGAAAC |
| | GGUGCAGCGGCUGUUGCCGGUGCUGUGCCAGGACCAUGGCCUGACCCCGGACCAAGUGGUGGCUAUCGCCAGCAAUCACGGCGGCAAGCA |
| | AGCGCUCGAAACGGUGCAGCGGCUGUUGCCGGUGCUGUGCCAGGACCAUGGCCUGACCCCGGACCAAGUGGUGGCUAUCGCCAGCAACGG |
| | UGGCGGCAAGCAAGCGCUCGAAACGGUGCAGCGGCUGUUGCCGGUGCUGUGCCAGGACCAUGGCCUGACCCCGGACCAAGUGGUGGCUA |
| | UCGCCAGCAACGGUGGCGGCAAGCAAGCGCUCGAAACGGUGCAGCGGCUGUUGCCGGUGCUGUGCCAGGACCAUGGCCUGACCCCGGACC |
| | AAGUGGUGGCUAUCGCCAGCAAUCACGGCGGCAAGCAAGCGCUCGAAACGGUGCAGCGGCUGUUGCCGGUGCUGUGCCAGGACCAUGGC |
| | CUGACCCCGGACCAAGUGGUGGCUAUCGCCAGCAACAUUGGCGGCAAGCAAGCGCUCGAAACGGUGCAGCGGCUGUUGCCGGUGCUGUG |
| | CCAGGACCAUGGCCUGACCCCGGACCAAGUGGUGGCUAUCGCCAGCAACAUUGGCGGCAAGCAAGCGCUCGAAACGGUGCAGCGGCUGUU |
| | GCCGGUGCUGUGCCAGGACCAUGGCCUGACCCCGGACCAAGUGGUGGCUAUCGCCAGCAACAUUGGCGGCAAGCAAGCGCUCGAAACGGU |
| | GCAGCGGCUGUUGCCGGUGCUGUGCCAGGACCAUGGCCUGACUCCGGACCAAGUGGUGGCUAUCGCCAGCCACGAUGGCGGCAAGCAAGC |
| | GCUCGAAACGGUGCAGCGGCUGUUGCCGGUGCUGUGCCAGGACCAUGGCCUGACCCCGGACCAAGUGGUGGCUAUCGCCAGCAACAUUG |
| | GCGGCAAGCAAGCGCUCGAAACGGUGCAGCGGCUGUUGCCGGUGCUGUGCCAGGACCAUGGCCUGACUCCGGACCAAGUGGUGGCUAUC |
| | GCCAGCCACGAUGGCGGCAAGCAAGCGCUCGAAACGGUGCAGCGGCUGUUGCCGGUGCUGUGCCAGGACCAUGGCCUGACCCCGGACCAA |
| | GUGGUGGCUAUCGCCAGCAACAUUGGCGGCAAGCAAGCGCUCGAAACGGUGCAGCGGCUGUUGCCGGUGCUGUGCCAGGACCAUGGCCU |
| | GACCCCGGACCAAGUGGUGGCUAUCGCCAGCAAUCACGGCGGCAAGCAAGCGCUCGAAACGGUGCAGCGGCUGUUGCCGGUGCUGUGCCA |
| | GGACCAUGGCCUGACCCCGGACCAAGUGGUGGCUAUCGCCAGCAACGGUGGCGGCAAGCAAGCGCUCGAAACGGUGCAGCGGCUGUUGCC |
| | GGUGCUGUGCCAGGACCAUGGCCUGACCCCGGACCAAGUGGUGGCUAUCGCCAGCAACGGUGGCGGCAAGCAAGCGCUCGAAACGGUGCA |
| | GCGGCUGUUGCCGGUGCUGUGCCAGGACCAUGGCCUGACCCCGGACCAAGUGGUGGCUAUCGCCAGCAACGGUGGCGGCAAGCAAGCGC |
| | UCGAAACGGUGCAGCGGCUGUUGCCGGUGCUGUGCCAGGACCAUGGCCUGACCCCGGACCAAGUGGUGGCUAUCGCCAGCAACAUUGGC |
| | GGCAAGCAAGCGCUCGAAACGGUGCAGCGGCUGUUGCCGGUGCUGUGCCAGGACCAUGGCCUGACUCCGGACCAAGUGGUGGCUAUCGC |
| | CAGCCACGAUGGCGGCAAGCAAGCGCUCGAAACGGUGCAGCGGCUGUUGCCGGUGCUGUGCCAGGACCAUGGCCUGACCCCGGACCAAGU |
| | GGUGGCUAUCGCCAGCAACAUUGGCGGCAAGCAAGCGCUCGAAACGGUGCAGCGGCUGUUGCCGGUGCUGUGCCAGGACCAUGGCCUGA |
| | CCCCGGACCAAGUGGUGGCUAUCGCCAGCAACGGUGGCGGCAAGCAAGCGCUCGAAACGGUGCAGCGGCUGUUGCCGGUGCUGUGCCAG |
| | GACCAUGGCCUGACUCCGGACCAAGUGGUGGCUAUCGCCAGCCACGAUGGCGGCAAGCAAGCGCUCGAAACGGUGCAGCGGCUGUUGCCG |
| | GUGCUGUGCCAGGACCAUGGCCUGACCCCGGACCAAGUGGUGGCUAUCGCCAGCAACGGUGGCGGCAAGCAAGCGCUCGAAACGGUGCAG |
| | CGGCUGUUGCCGGUGCUGUGCCAGGACCAUGGCCUGACCCCGGACCAAGUGGUGGCUAUCGCCAGCAAUCACGGCGGCAAGCAAGCGCUC |
| | GAAACGGUGCAGCGGCUGUUGCCGGUGCUGUGCCAGGACCAUGGCGCUAUCGCCAGCAACAAUGGCGGCAAGCAAGCGCUCGAAAGCAU |
| | UGUGGCCCAGCUGAGCCGGCCUGAUCCGGCGUUGGCCGCGUUAACCAACGACCACCUCGUCGCCUUGGCCUGCCUCGGCGGACGUCCUGCC |
| | AUGGAUGCAGUGAAAAAGGGAUUGCCGCACGCGCCGGAAUUGAUCAGAAGAGUCAAUCGCCGUAUUGGCGAACGCACGUCCCAUCGCGUU |
| | GCCAGGGACCCAAAGAAGAAGCGCAAAGUGGAUCCUAAAAAGAAAAGAAAGGUAGGCGGCCGCGGGGGAGGCGGUUCCGGUGGCGGCGGA |
| | AGCGGAGGUGGAGGAUCAGGGCCGGCCGGAGGAGGUGGAAGCGGAGGAGGAGGAAGCGGAGGAGGAGGUAGCGGACCUAAGAAAAAGAG |
| | GAAGGUGGCGGCCGCUGGAUCCCCUUCAGGGCAGAUCAGCAACCAGGCCCUGGCUCUGGCCCCUAGCUCCGCUCCAGUGCUGGCCCAGACU |
| | AUGGUGCCCUCUAGUGCUAUGGUGCCUCUGGCCCAGCCACCUGCUCCAGCCCCUGUGCUGACCCCAGGACCACCCCAGUCACUGAGCGCUCC |
| | AGUGCCCAAGUCUACACAGGCCGGCGAGGGGACUCUGAGUGAAGCUCUGCUGCACCUGCAGUUCGACGCUGAUGAGGACCUGGGAGCUCU |
| | GCUGGGGAACAGCACCGAUCCCGGAGUGUUCACAGAUCUGGCCUCCGUGGACAACUCUGAGUUUCAGCAGCUGCUGAAUCAGGGCGUGUC |
| | CAUGUCUCAUAGUACAGCCGAACCAAUGCUGAUGGAGUACCCCGAAGCCAUUACCCGGCUGGUGACCGGCAGCCAGCGGCCCCCCGACCCCG |
| | CUCCAACUCCCCUGGGAACCAGCGGCCUGCCUAAUGGGCUGUCCGGAGAUGAAGACUUCUCAAGCAUCGCUGAUAUGGACUUUAGUGCCCU |
| | GCUGUCACAGAUUUCCUCUAGUGGGCAGGGAGGAGGUGGAAGCGGCUUCAGCGUGGACACCAGUGCCCUGCUGGACCUGUUCAGCCCCUC |
| | GGUGACCGUGCCCGACAUGAGCCUGCCUGACCUUGACAGCAGCCUGGCCAGUAUCCAAGAGCUCCUGUCUCCCCAGGAGCCCCCCAGGCCUC |
| | CCGAGGCAGAGAACAGCAGCCCGGAUUCAGGGAAGCAGCUGGUGCACUACACAGCGCAGCCGCUGUUCCUGCUGGACCCCGGCUCCGUGGA |
| | CACCGGGAGCAACGACCUGCCGGUGCUGUUUGAGCUGGGAGAGGGCUCCUACUUCUCCGAAGGGGACGGCUUCGCCGAGGACCCCACCAUC |
| | UCCCUGCUGACAGGCUCGGAGCCUCCCAAAGCCAAGGACCCCACUGUCUCCAUCGAUUGA |

TABLE 7
Elements of mRNA sequences
Nucleotides 1-69: FLAG sequence (double underlined)
Nucleotides 91-162: 3x NLS sequence (dotted underlined)

| mRNA | TALES (bold) | p65/HSF-1 (underlined) |
|---|---|---|
| mTZ1 (SEQ ID NO: 31) | 649-2178 | 2620-3461 |
| mTZ2 (SEQ ID NO: 32) | 649-2382 | 2824-3666 |
| mTZ3 (SEQ ID NO: 33) | 649-2280 | 2722-3564 |
| mTZ4 (SEQ ID NO: 34) | 649-2281 | 2723-3565 |
| mTZ5 (SEQ ID NO: 35) | 649-3177 | 3619-4461 |
| mTZ6 (SEQ ID NO: 36) | 649-3177 | 3619-4461 |
| mTZ7 (SEQ ID NO: 37) | 649-3177 | 3619-4461 |
| mTZ8 (SEQ ID NO: 38) | 649-3177 | 3619-4461 |
| mTZ9 (SEQ ID NO: 39) | 649-2871 | 3313-4155 |
| mTZ10 (SEQ ID NO: 40) | 649-3279 | 3721-4563 |
| mTR1 (SEQ ID NO: 41) | 649-2994 | 3436-4278 |
| mTR2 (SEQ ID NO: 42) | 649-3585 | 4027-4869 |
| mTR3 (SEQ ID NO: 43) | 649-2733 | 3175-4017 |
| mTR4 (SEQ ID NO: 44) | 649-2664 | 3106-3948 |
| mTR5 (SEQ ID NO: 45) | 649-3585 | 4027-4869 |
| mTR6 (SEQ ID NO: 46) | 649-2973 | 3415-4257 |
| mTR7 (SEQ ID NO: 47) | 649-3483 | 3925-4767 |
| mTR8 (SEQ ID NO: 48) | 649-3483 | 3925-4767 |
| mTR9 (SEQ ID NO: 49) | 649-2463 | 2906-3747 |
| mTR10 (SEQ ID NO: 50) | 649-2667 | 3109-3951 |
| mTG1 (SEQ ID NO: 51) | 649-3585 | 4027-4869 |
| mTG2 (SEQ ID NO: 52) | 649-3177 | 3619-4461 |
| mTG3 (SEQ ID NO: 53) | 649-3177 | 3619-4461 |
| mTG4 (SEQ ID NO: 54) | 649-2565 | 3007-3849 |
| mTG5 (SEQ ID NO: 55) | 649-2565 | 3007-3849 |
| mTG6 (SEQ ID NO: 56) | 649-2769 | 3211-4053 |
| mTG7 (SEQ ID NO: 57) | 649-3177 | 3619-4461 |
| mTG8 (SEQ ID NO: 58) | 649-2157 | 2599-3441 |
| mTG9 (SEQ ID NO: 59) | 649-2565 | 3007-3849 |
| mTG10 (SEQ ID NO: 60) | 649-3177 | 3619-4461 |

TABLE 8
Plasmids used in this study

| Casilio Activation System: | Description |
|---|---|
| pLenti-HA-dCas9-2A-EGFP-2A- | lentiviral vector constitutively expressing HA- |
| Blast | dCas9-2A-EGFP |
| pLX-3xFLAG-4xNLS-PUFa- | lentiviral vector constitutively expressing 3xFLAG- |
| 2xNLS-p65HSF1-IRES-Hygro | PUFa-p65HSF1 transactivator |
| | sgRNA-target sequence (added 5′ g in lower case) |
| sgRNAs: | Target sequence PAM |
| pLentiGuide-sgBZLF1-1-5xPBSa | GCAAAGATAGCAAAGGTGGC CGG |
| (sgRNA1) | |
| pLentiGuide-sgBZLF1-2-5xPBSa | gCAGCCTCCTCTGTGATGTCA TGG |
| (sgRNA2) | |
| pLentiGuide-sgBZLF1-3-5xPBSa | GAAACTATGCATGAGCCAC AGG |
| (sgRNA3) | |
| pLentiGuide-sgBZLF1-4-5xPBSa | gCAGAAGTGTCTAAAATAAGC TGG |
| (sgRNA4) | |
| Inducible 3xFLAG-PUFa-p65HSF | |
| piggyBac plasmid: | Description |
| PB-Hygro_2a_rtTA3-3xFLAG- | piggyBac transposon vector expressing rtTA3 and |
| 4xNLS-PUFa-2xNLS-p65HSF1 | Tet-On 3xFLAG-PUFa-p65HSF transactivator |
| TALE plasmids | Target sequence |
| pcDNA3.1-3xFLAG- | TAGCAAAGGTGGCCGG |
| 4xNLS_TALE-BZLP1- | |
| 1_2xNLS_p65HSF1 (TZ1) | |
| pcDNA3.1-3xFLAG- | TCCTCTGTGATGTCATGG |
| 4xNLS_TALE-BZLP1- | |
| 2_2xNLS_p65HSF1 (TZ2) | |
| pcDNA3.1-3xFLAG- | TATGCATGAGCCACAGG |
| 4xNLS_TALE-BZLP1- | |
| 3_2xNLS_p65HSF1 (TZ3) | |
| pcDNA3.1-3xFLAG- | TGTCTAAAATAAGCTGG |
| 4xNLS_TALE-BZLP1- | |
| 4_2xNLS_p65HSF1 (TZ4) | |

TABLE 9
sgRNA-and TALE-binding sequences in the
BZLF1 promoter
sgRBA sequences

| | |
|---|---|
| sg1 | 5′ - ggcca ccttt gctat cttt - 3′ |
| sg2 | 5′ - tgaca tcaca gagga ggc - 3′ |
| sg3 | 5′ - aaact atgca tgagc cac - 3′ |
| sg4 | 5′ - gctta tttta gacac ttc - 3′ |

TALE targeted sequences

| | |
|---|---|
| TZ1 | agcaaaggtggccgg |
| TZ2 | cctctgtgatgtcatgg |
| TZ3 | atgcatgagccacagg |
| TZ4 | gtctaaaataagctgg |

TABLE 10
amino acid sequences of the TALEs and RNA sequences of TZ3 mRNA

| | |
|---|---|
| TZ1 | MDYKDHDGDYKDHDIDYKDDDDKIDGGGGSDPKKKRKVDPKKKRKVDP |
| amino | KKKRKVGSTGSRNDGGGGSGGGGSGGGGSGRAVDLRTLGYSQQQQEKIK |
| acid | PKVRSTVAQHHEALVGHGFTHAHIVALSQHPAALGTVAVTYQHIITALPEA |
| sequence | THEDIVGVGKQWSGARALEALLTDAGELRGPPLQLDTGQLVKIAKRGGVT |
| | AMEAVHASRNALTGAPLNLTPDQVVAIASNIGGKQALETVQRLLPVLCQD |
| | HGLTPDQVVAIASNNGGKQALETVQRLLPVLCQDHGLTPDQVVAIASHDG |
| | GKQALETVQRLLPVLCQDHGLTPDQVVAIASNIGGKQALETVQRLLPVLC |
| | QDHGLTPDQVVAIASNIGGKQALETVQRLLPVLCQDHGLTPDQVVAIASNI |
| | GGKQALETVQRLLPVLCQDHGLTPDQVVAIASNNGGKQALETVQRLLPVL |
| | CQDHGLTPDQVVAIASNNGGKQALETVQRLLPVLCQDHGLTPDQVVAIAS |
| | NGGGKQALETVQRLLPVLCQDHGLTPDQVVAIASNNGGKQALETVQRLLP |
| | VLCQDHGLTPDQVVAIASNNGGKQALETVQRLLPVLCQDHGLTPDQVVAI |
| | ASHDGGKQALETVQRLLPVLCQDHGLTPDQVVAIASHDGGKQALETVQR |
| | LLPVLCQDHGLTPDQVVAIASNNGGKQALETVQRLLPVLCQDHGLTPDQV |
| | VAIASNNGGKQALESIVAQLSRPDPALAALTNDHLVALACLGGRPAMDAV |
| | KKGLPHAPELIRRVNRRIGERTSHRVARDPKKKRKVDPKKKRKVGGRGGG |
| | GSGGGGSGGGGSGPAGGGGSGGGGSGGGGSGPKKKRKVAAAGSPSGQIS |
| | NQALALAPSSAPVLAQTMVPSSAMVPLAQPPAPAPVLTPGPPQSLSAPVPK |
| | STQAGEGTLSEALLHLQFDADEDLGALLGNSTDPGVFTDLASVDNSEFQQL |
| | LNQGVSMSHSTAEPMLMEYPEAITRLVTGSQRPPDPAPTPLGTSGLPNGLS |
| | GDEDFSSIADMDFSALLSQISSSGQGGGGSGFSVDTSALLDLESPSVTVPDM |
| | SLPDLDSSLASIQELLSPQEPPRPPEAENSSPDSGKQLVHYTAQPLFLLDPGS |
| | VDTGSNDLPVLFELGEGSYFSEGDGFAEDPTISLLTGSEPPKAKDPTVSID* |
| TZ2 | MDYKDHDGDYKDHDIDYKDDDDKIDGGGGSDPKKKRKVDPKKKRKVDP |
| amino | KKKRKVGSTGSRNDGGGGSGGGGSGGGGSGRAVDLRILGYSQQQQEKIK |
| acid | PKVRSTVAQHHEALVGHGFTHAHIVALSQHPAALGTVAVTYQHIITALPEA |
| sequence | THEDIVGVGKQWSGARALEALLTDAGELRGPPLQLDTGQLVKIAKRGGVT |
| | AMEAVHASRNALTGAPLNLTPDQVVAIASNIGGKQALETVQRLLPVLCQD |
| | HGLTPDQVVAIASNNGGKQALETVQRLLPVLCQDHGLTPDQVVAIASHDG |
| | GKQALETVQRLLPVLCQDHGLTPDQVVAIASNIGGKQALETVQRLLPVLC |
| | QDHGLTPDQVVAIASNIGGKQALETVQRLLPVLCQDHGLTPDQVVAIASNI |
| | GGKQALETVQRLLPVLCQDHGLTPDQVVAIASNNGGKQALETVQRLLPVL |
| | CQDHGLTPDQVVAIASNNGGKQALETVQRLLPVLCQDHGLTPDQVVAIAS |
| | NGGGKQALETVQRLLPVLCQDHGLTPDQVVAIASNNGGKQALETVQRLLP |
| | VLCQDHGLTPDQVVAIASNNGGKQALETVQRLLPVLCQDHGLTPDQVVAI |
| | ASHDGGKQALETVQRLLPVLCQDHGLTPDQVVAIASHDGGKQALETVQR |
| | LLPVLCQDHGLTPDQVVAIASNNGGKQALETVQRLLPVLCQDHGLTPDQV |
| | VAIASNNGGKQALESIVAQLSRPDPALAALTNDHLVALACLGGRPAMDAV |
| | KKGLPHAPELIRRVNRRIGERTSHRVARDPKKKRKVDPKKKRKVGGRGGG |
| | GSGGGGSGGGGSGPAGGGGSGGGGSGGGGSGPKKKRKVAAAGSPSGQIS |
| | NQALALAPSSAPVLAQTMVPSSMVPLAQPPAPAPVLTPGPPQSLSAPVPKS |
| | TQAGEGTLSEALLHLQFDADEDLGALLGNSTDPGVFTDLASVDNSEFQQLL |
| | NQGVSMSHSTAEPMLMEYPEAITRLVTGSQRPPDPAPTPLGTSGLPNGLSG |
| | DEDFSSIADMDFSALLSQISSSGQGGGGSGFSVDTSALLDLFSPSVTVPDMS |
| | LPDLDSSLASIQELLSPQEPPRPPEAENSSPDSGKQLVHYTAQPLFLLDPGSV |
| | DTGSNDLPVLFELGEGSYFSEGDGFAEDPTISLLTGSEPPKAKDPTVSID* |
| TZ3 | MDYKDHDGDYKDHDIDYKDDDDKIDGGGGSDPKKKRKVDPKKKRKVDP |
| amino | KKKRKVGS |
| acid | TGSRNDGGGGSGGGGSGGGGSGRAVDLRTLGYSQQQQEKIKPKVRSTVA |
| sequence | QHHEALVG |
| | HGFTHAHIVALSQHPAALGTVAVTYQHIITALPEATHEDIVGVGKQWSGAR |
| | ALEALLTD |
| | AGELRGPPLQLDTGQLVKIAKRGGVTAMEAVHASRNALTGAPLNLTPDQV |
| | VAIASNIGGKQALETVQRLLPVLCQDHGLTPDQVVAIASNGGGKQALETV |
| | QRLLPVLCQDHGLTPDQVVAIASNNGGKQALETVQRLLPVLCQDHGLTPD |
| | QVVAIASHDGGKQALETVQRLLPVLCQDHGLTPDQVVAIASNIGGKQALE |
| | TVQRLLPVLCQDHGLTPDQVVAIASNGGGKQALETVQRLLPVLCQDHGLT |
| | PDQVVAIASNNGGKQALETVQRLLPVLCQDHGLTPDQVVAIASNIGGKQA |
| | LETVQRLLPVLCQDHGLTPDQVVAIASNNGGKQALETVQRLLPVLCQDHG |
| | LTPDQVVAIASHDGGKQALETVQRLLPVLCQDHGLTPDQVVAIASHDGGK |
| | QALETVQRLLPVLCQDHGLTPDQVVAIASNIGGKQALETVQRLLPVLCQD |
| | HGLTPDQVVAIASHDGGKQALETVQRLLPVLCQDHGLTPDQVVAIASNIG |
| | GKQALETVQRLLPVLCQDHGLTPDQVVAIASNNGGKQALETVQRLLPVLC |
| | QDHGLTPDQVVAIASNNGGKQALESIVAQLSRPDPALAALINDHLVALAC |
| | LGGRPAMDAVKKGLPHAPELIRRVNRRIGERTSHRVARDPKKKRKVDPKK |
| | KRKVGGRGGGGSGGGGSGGGGSGPAGGGGSGGGGSGGGGSGPKKKRKV |
| | AAAGSPSGQISNQALALAPSSAPVLAQTMVPSSAMVPLAQPPAPAPVLTPG |
| | PPQSLSAPVPKSTQAGEGTLSEALLHLQFDADEDLGALLGNSTDPGVFTDL |
| | ASVDNSEFQQLLNQGVSMSHSTAEPMLMEYPEAITRLVTGSQRPPDPAPTP |
| | LGTSGLPNGLSGDEDFSSIADMDFSALLSQISSSGQGGGGSGFSVDTSALLD |
| | LFSPSVTVPDMSLPDLDSSLASIQELLSPQEPPRPPEAENSSPDSGKQLVHYT |
| | AQPLFLLDPGSVDTGSNDLPVLFELGEGSYFSEGDGFAEDPT |
| | ISLLTGSEPPKAKDPTVSID* |
| TZ4 | MDYKDHDGDYKDHDIDYKDDDDKIDGGGGSDPKKKRKVDPKKKRKVDP |
| amino | KKKRKVGSTGSRNDGGGGSGGGGSGGGGSGRAVDLRTLGYSQQQQEKIK |
| acid | PKVRSTVAQHHEALVGHGFTHAHIVALSQHPAALGTVAVTYQHIITALPEA |
| sequence | THEDIVGVGKQWSGARALEALLTDAGELRGPPLQLDTGQLVKIAKRGGVT |
| | AMEAVHASRNALTGAPLNLTPDQVVAIASNNGGKQALETVQRLLPVLCQ |
| | DHGLTPDQVVAIASNGGGKQALETVQRLLPVLCQDHGLTPDQVVAIASHD |
| | GGKQALETVQRLLPVLCQDHGLTPDQVVAIASNGGGKQALETVQRLLPVL |
| | CQDHGLTPDQVVAIASNIGGKQALETVQRLLPVLCQDHGLTPDQVVAIAS |
| | NIGGKQALETVQRLLPVLCQDHGLTPDQVVAIASNIGGKQALETVQRLLPV |
| | LCQDHGLTPDQVVAIASNIGGKQALETVQRLLPVLCQDHGLTPDQVVAIAS |
| | NGGGKQALETVQRLLPVLCQDHGLTPDQVVAIASNIGGKQALETVQRLLP |
| | VLCQDHGLTPDQVVAIASNIGGKQALETVQRLLPVLCQDHGLTPDQVVAI |
| | ASNNGGKQALETVQRLLPVLCQDHGLTPDQVVAIASHDGGKQALETVQR |
| | LLPVLCQDHGLTPDQVVAIASNGGGKQALETVQRLLPVLCQDHGLTPDQV |
| | VAIASNNGGKQALETVQRLLPVLCQDHGLTPDQVVAIASNNGGKQALESI |
| | VAQLSRPDPALAALTNDHLVALACLGGRPAMDAVKKGLPHAPELIRRVNR |
| | RIGERTSHRVARDPKKKRKVDPKKKRKVGGRGGGGSGGGGSGGGGSGPA |
| | GGGGSGGGGSGGGGSGPKKKRKVAAAGSPSGQISNQALALAPSSAPVLAQ |
| | TMVPSSAMVPLAQPPAPAPVLTPGPPQSLSAPVPKSTQAGEGTLSEALLHL |
| | QFDADEDLGALLGNSTDPGVFTDLASVDNSEFQQLLNQGVSMSHSTAEPM |
| | LMEYPEAITRLVTGSQRPPDPAPTPLGTSGLPNGLSGDEDFSSIADMDFSAL |
| | LSQISSSGQGGGGSGFSVDTSALLDLFSPSVTVPDMSLPDLDSSLASIQELLS |
| | PQEPPRPPEAENSSPDSGKQLVHYTAQPLFLLDPGSVDTGSNDLPVLFELGE |
| | GSYFSEGDGFAEDPTI 45 SLLTGSEPPKAKDPTVSID* |
| TZ3 | AUGGACUACAAGGAUCACGACGGUGACUAUAAGGAUCAUGACAUCGA |
| mRNA | CUAUAAGGACGAUGACGAUAAGAUCGAUGGCGGAGGCGGAUCUGAUC |
| | CAAAAAAGAAGAGAAAGGUAGAUCCAAAAAAGAAGAGAAAGGUAGA |
| | UCCAAAAAAGAAGAGAAAGGUAGGAUCUACCGGAUCUAGAAACGAU |
| | GGUGGUGGUGGAAGCGGGGGUGGGGGCAGCGGUGGAGGGGGAAGCG |
| | GGCGCGCCGUGGAUCUACGCACGCUCGGCUACAGUCAGCAGCAGCAA |
| | GAGAAGAUCAAACCGAAGGUGCGUUCGACAGUGGCGCAGCACCACGA |
| | GGCACUGGUGGGCCAUGGGUUUACACACGCGCACAUCGUUGCGCUCA |
| | GCCAACACCCGGCAGCGUUAGGGACCGUCGCUGUCACGUAUCAGCAC |
| | AUAAUCACGGCGUUGCCAGAGGCGACACACGAAGACAUCGUUGGCGU |
| | CGGCAAACAGUGGUCCGGCGCACGCGCCCUGGAGGCCUUGCUCACGG |
| | AUGCGGGGGAGUUGAGAGGUCCGCCGUUACAGUUGGACACAGGCCAA |
| | CUUGUGAAGAUUGCAAAACGUGGCGGCGUGACCGCAAUGGAGGCAGU |
| | GCAUGCAUCGCGCAAUGCACUGACGGGUGCCCCCCUGAACCUGACCC |
| | CGGACCAAGUGGUGGCUAUCGCCAGCAACAUUGGCGGCAAGCAAGCG |
| | CUCGAAACGGUGCAGCGGCUGUUGCCGGUGCUGUGCCAGGACCAUGG |
| | CCUGACCCCGGACCAAGUGGUGGCUAUCGCCAGCAACGGUGGCGGCA |
| | AGCAAGCGCUCGAAACGGUGCAGCGGCUGUUGCCGGUGCUGUGCCAG |
| | GACCAUGGCCUGACCCCGGACCAAGUGGUGGCUAUCGCCAGCAAUCA |
| | CGGCGGCAAGCAAGCGCUCGAAACGGUGCAGCGGCUGUUGCCGGUGC |
| | UGUGCCAGGACCAUGGCCUGACUCCGGACCAAGUGGUGGCUAUCGCC |
| | AGCCACGAUGGCGGCAAGCAAGCGCUCGAAACGGUGCAGCGGCUGUU |
| | GCCGGUGCUGUGCCAGGACCAUGGCCUGACCCCGGACCAAGUGGUGG |
| | CUAUCGCCAGCAACAUUGGCGGCAAGCAAGCGCUCGAAACGGUGCAG |
| | CGGCUGUUGCCGGUGCUGUGCCAGGACCAUGGCCUGACCCCGGACCA |
| | AGUGGUGGCUAUCGCCAGCAACGGUGGCGGCAAGCAAGCGCUCGAAA |
| | CGGUGCAGCGGCUGUUGCCGGUGCUGUGCCAGGACCAUGGCCUGACC |
| | CCGGACCAAGUGGUGGCUAUCGCCAGCAAUCACGGCGGCAAGCAAGC |
| | GCUCGAAACGGUGCAGCGGCUGUUGCCGGUGCUGUGCCAGGACCAUG |
| | GCCUGACCCCGGACCAAGUGGUGGCUAUCGCCAGCAACAUUGGCGGC |
| | AAGCAAGCGCUCGAAACGGUGCAGCGGCUGUUGCCGGUGCUGUGCCA |
| | GGACCAUGGCCUGACCCCGGACCAAGUGGUGGCUAUCGCCAGCAAUC |
| | ACGGCGGCAAGCAAGCGCUCGAAACGGUGCAGCGGCUGUUGCCGGUG |
| | CUGUGCCAGGACCAUGGCCUGACUCCGGACCAAGUGGUGGCUAUCGC |
| | CAGCCACGAUGGCGGCAAGCAAGCGCUCGAAACGGUGCAGCGGCUGU |
| | UGCCGGUGCUGUGCCAGGACCAUGGCCUGACCCCGGACCAAGUGGUG |
| | GCUAUCGCCAGCCACGAUGGCGGCAAGCAAGCGCUCGAAACGGUGCA |
| | GCGGCUGUUGCCGGUGCUGUGCCAGGACCAUGGCCUGACCCCGGACC |
| | AAGUGGUGGCUAUCGCCAGCAACAUUGGCGGCAAGCAAGCGCUCGAA |
| | ACGGUGCAGCGGCUGUUGCCGGUGCUGUGCCAGGACCAUGGCCUGAC |
| | UCCGGACCAAGUGGUGGCUAUCGCCAGCCACGAUGGCGGCAAGCAAG |
| | CGCUCGAAACGGUGCAGCGGCUGUUGCCGGUGCUGUGCCAGGACCAU |
| | GGCCUGACCCCGGACCAAGUGGUGGCUAUCGCCAGCAACAUUGGCGG |
| | CAAGCAAGCGCUCGAAACGGUGCAGCGGCUGUUGCCGGUGCUGUGCC |
| | AGGACCAUGGCCUGACCCCGGACCAAGUGGUGGCUAUCGCCAGCAAU |
| | CACGGGGGCAAGCAAGCGCUCGAAACGGUGCAGCGGCUGUUGCCGGU |
| | GCUGUGCCAGGACCAUGGCCUGACCCCGGACCAAGUGGUGGCUAUCG |
| | CCAGCAAUCACGGCGGCAAGCAAGCGCUCGAAAGCAUUGUGGCCCAG |
| | CUGAGCCGGCCUGAUCCGGCGUUGGCCGCGUUGACCAACGACCACCU |
| | CGUCGCCUUGGCCUGCCUCGGCGGACGUCCUGCCAUGGAUGCAGUGA |
| | AAAAGGGAUUGCCGCACGCGCCGGAAUUGAUCAGAAGAGUCAAUCGC |
| | CGUAUUGGCGAACGCACGUCCCAUCGCGUUGCCAGGGACCCAAAGAA |
| | GAAGCGCAAAGUGGAUCCUAAAAAGAAAAGAAAGGUAGGCGGCCGCG |
| | GGGGAGGCGGUUCCGGUGGCGGCGGAAGCGGAGGUGGAGGAUCAGG |
| | GCCGGCCGGAGGAGGUGGAAGCGGAGGAGGAGGAAGCGGAGGAGGA |
| | GGUAGCGGACCUAAGAAAAAGAGGAAGGUGGCGGCCGCUGGAUCCCC |
| | UUCAGGGCAGAUCAGCAACCAGGCCCUGGCUCUGGCCCCUAGCUCCG |
| | CUCCAGUGCUGGCCCAGACUAUGGUGCCCUCUAGUGCUAUGGUGCCU |
| | CUGGCCCAGCCACCUGCUCCAGCCCCUGUGCUGACCCCAGGACCACCC |
| | CAGUCACUGAGCGCUCCAGUGCCCAAGUCUACACAGGCCGGCGAGGG |
| | GACUCUGAGUGAAGCUCUGCUGCACCUGCAGUUCGACGCUGAUGAGG |
| | ACCUGGGAGCUCUGCUGGGGAACAGCACCGAUCCCGGAGUGUUCACA |
| | GAUCUGGCCUCCGUGGACAACUCUGAGUUUCAGCAGCUGCUGAAUCA |
| | GGGCGUGUCCAUGUCUCAUAGUACAGCCGAACCAAUGCUGAUGGAGU |
| | ACCCCGAAGCCAUUACCCGGCUGGUGACCGGCAGCCAGCGGCCCCCCG |
| | ACCCCGCUCCAACUCCCCUGGGAACCAGCGGCCUGCCUAAUGGGCUG |
| | UCCGGAGAUGAAGACUUCUCAAGCAUCGCUGAUAUGGACUUUAGUGC |
| | CCUGCUGUCACAGAUUUCCUCUAGUGGGCAGGGAGGAGGUGGAAGCG |
| | GCUUCAGCGUGGACACCAGUGCCCUGCUGGACCUGUUCAGCCCCUCG |
| | GUGACCGUGCCCGACAUGAGCCUGCCUGACCUUGACAGCAGCCUGGC |
| | CAGUAUCCAAGAGCUCCUGUCUCCCCAGGAGCCCCCCAGGCCUCCCGA |
| | GGCAGAGAACAGCAGCCCGGAUUCAGGGAAGCAGCUGGUGCACUACA |
| | CAGCGCAGCCGCUGUUCCUGCUGGACCCCGGCUCCGUGGACACCGGG |
| | AGCAACGACCUGCCGGUGCUGUUUGAGCUGGGAGAGGGCUCCUACUU |
| | CUCCGAAGGGGACGGCUUCGCCGAGGACCCCACCAUCUCCCUGCUGA |
| | CAGGCUCGGAGCCUCCCAAAGCCAAGGACCCCACUGUCUCCAUCGAU |
| | UGA |

TABLE 11
primer sequences for quantitative real-time PCR

| Genes | Primers for qRT-PCR |
|---|---|
| BRLF1 | For 5′-GCATGGGCGGGACAATCGCAATATAA -3′ |
| | Rev 5′-CCAGCCAGATGTTCAGGAACCAAA -3′ |
| BZLF1 | For 5′- TACAAGAATCGGGTGGCTTC -3′ |
| | Rev 5′- GCACATCTGCTTCAACAGGA -3 |
| BMRF1 | For 5′ - CACTGCGGTGGAGGTAGAG -3′ |
| | Rev 5′- GGTGGTGTGCCATACAAGG -3′ |
| BLRF2 | For 5′ - ACTGAAGCCCAGGACCAGTTCTA -3′ |
| | Rev 5′ - TAAGACAAGCGTCAGAAGTGCCCA -3′ |
| BGLF4 | For 5′- GCTGACTCCACCCACAAAAT -3′ |
| | Rev 5′- GAGGTCAGGCCCATGTCTAA -3′ |
| GAPDH | For 5′ - GAAGGTGAAGGTCGGAGTCA -3′ |
| | Rev 5′ - TGACAAGCTTCCGGTTCTC -3′ |

1. A nucleic acid comprising a polynucleotide sequence encoding a fusion protein comprising (i) at least one nuclear-localization signal (NLS); (ii) a transcription activator-like effector (TA LE) targeting Epstein-Barr virus (EBV) BZLF1, BRLF1, or BGLF4 promoter sequence; and (iii) a transactivation domain.

2. The nucleic acid of claim 1, which is RNA.

3. The nucleic acid of claim 2, wherein U residues of the RNA are replaced with pseudouridine.

4. The nucleic acid of claim 1, which is DNA and comprises an expression cassette comprising the polynucleotide sequence operably linked to a promoter sequence.

5. The nucleic acid of any one of claims 1-4, wherein the TA LE is encoded by a nucleotide sequence having at least 90% sequence identity to segment 649-2280 of SEQ ID NO: 33.

6. The nucleic acid of any one of claims 1-5, wherein the transactivation domain is encoded by a nucleotide sequence having at 1 east 90% sequence identity to segment 2722-3564 of SEQ ID NO: 33.

7. The nucleic acid of any one of claims 1-6, wherein the at least one NLS is encoded by a nucleotide sequence having at least 90% sequence identity to segment 91-114 of SEQ ID NO: 33.

8. The nucleic acid of any one of claims 1-7, wherein the fusion protein further comprises an epitope tag.

9. The nucleic acid of any one of claims 1-8, wherein the fusion protein comprises, from its N-terminus to its C-terminus, a FLAG encoded by segment 1-69 of SEQ ID NO: 33, at least one NLS encoded by segment 91-162 of SEQ ID NO: 33, a TALE encoded by segment 649-2280 of SEQ ID NO: 33, and a transactivation domain encoded by segment 2722-3564 of SEQ ID NO: 33.

10. The nucleic acid of any one of claims 1-8, wherein the polynucleotide sequence is set forth in any one of SEQ ID NOs: 31-60.

11. A composition for use in treating an EBV-associated disease in a subject, comprising an effective amount of (1) the nucleic acid of any one of claims 1-10; and (2) a physiologically acceptable excipient.

12. The composition of claim 11, wherein the nucleic acid is RNA.

13. The composition of claim 12, wherein U residues of the RNA are replaced with pseudouridine.

14. The composition of any one of claims 11-13, wherein the nucleic acid is present within lipid nanoparticles.

15. The composition of any one of claims 11-14, wherein the lipid nanoparticles comprise [(4-hydroxybutyl) azanediyl] di (hexan e-6,1-diyl) bis (2-hexyldecanoate) (ALC-0315), 1,2-distearoyl-sn-glycero-3-phosphocholine (DSPC), 1,2-dimyristoyl-rac-glycero-3-methoxypolyethylene glycol-2000 (DMG-PEG 2000), and cholesterol.

16. The composition of any one of claims 11-15, which is formulated for injection to the subject.

17. A method for treating an EBV-associated disease in a subject, comprising administering to the subject an effective amount of the composition of any one of claims 11-16.

18. The method of claim 17, wherein the EBV-associated disease is an EBV-associated cancer.

19. The method of claim 18, wherein the EBV-associated cancer is Burkitt lymphoma, Hodgkin's lymphoma, natural killer cell lymphoma, T-cell lymphoma, post-transplant lymphoma, nasopharyngeal carcinoma, or gastric cancer.

20. The method of any one of claims 17-19, wherein the nucleic acid is RNA.

21. The method of claim 20, wherein U residues of the RNA are replaced with pseudouridine.

22. The method of any one of claims 17-21, wherein the nucleic acid is present in lipid nanoparticles.

23. The method of claim 22, wherein the lipid nanoparticles comprise [(4-hydroxybutyl) azanediyl] di (hexane-6,1-diyl) bis(2-hexyldecanoate) (ALC-0315), 1, 2-distearoyl-sn-glycero-3-phosphocholine (D SPC), 1,2-dimyristoyl-rac-glycero-3-methoxypolyethylene glycol-2000 (DMG-PEG 2000), and cholesterol.

24. The method of any one of claims 17-23, wherein the administering comprises injection of the composition to the subject.

25. A kit for treating an EBV-associated disease in a subject comprising a first container containing a first composition comprising an effective amount of at least one of the nucleic acid of any one of claims 1-10 and a second container containing a second composition comprising an effective amount of at least one other anti-cancer therapeutic agent for the EBV-associated disease.

26. The kit of claim 25, wherein the nucleic acid is RNA and the polynucleotide sequence is set forth in any one of SEQ ID NOs: 31-60.

27. The kit of claim 25 or 26, wherein the nucleic acid is RNA and the polynucleotide sequence is set forth in any one of SEQ ID NOs: 51-60, and wherein the other anti-cancer agent comprises ganciclovir (GCV).

28. The kit of any one of claims 25-27, wherein the EBV-associated disease is an EBV-associated cancer, and wherein the second composition comprising an effective amount of at least one other anti-cancer therapeutic agent.

29. The kit of claim 28, wherein the EBV-associated cancer is Burkitt lymphoma, Hodgkin's lymphoma, natural killer cell lymphoma, T-cell lymphoma, post-transplant lymphoma, nasopharyngeal carcinoma, or gastric cancer.
